SEQUENCE LISTING (22 sequences)

SEQ ID NO: 1 - protein, 6 aa, Homo sapiens
DTTLLD

SEQ ID NO: 2 - protein, 6 aa, Homo sapiens
DASLFT

SEQ ID NO: 3 - protein, 5 aa, Homo sapiens
DEASG

SEQ ID NO: 4 - protein, 5 aa, Homo sapiens
DLGDK

SEQ ID NO: 5 - protein, 6 aa, Homo sapiens
DTTLLD

SEQ ID NO: 6 - protein, 6 aa, Homo sapiens
DNGSLA

SEQ ID NO: 7 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 8 - protein, 941 aa, Homo sapiens
CCAAGAGCTAAAAGAGAGCAAGGAGGAAACAACAGCAGCTCCAACCAGGGCAGCCTTCCTGAGAAGATGCAACCAATCCTGCTTCTGCTGGCCTTCCTCCTGCTGCCCAGGGCAGATGCAGGGGAGATCATCGGGGGACATGAGGCCAAGCCCCACTCCCGCCCCTACATGGCTTATCTTATGATCTGGGATCAGAAGTCTCTGAAGAGGTGCGGTGGCTTCCTGATACGAGACGACTTCGTGCTGACAGCTGCTCACTGTTGGGGAAGCTCCATAAATGTCACCTTGGGGGCCCACAATATCAAAGAACAGGAGCCGACCCAGCAGTTTATCCCTGTGAAAAGACCCATCCCCCATCCAGCCTATAATCCTAAGAACTTCTCCAACGACATCATGCTACTGCAGCTGGAGAGAAAGGCCAAGCGGACCAGAGCTGTGCAGCCCCTCAGGCTACCTAGCAACAAGGCCCAGGTGAAGCCAGGGCAGACATGCAGTGTGGCCGGCTGGGGGCAGACGGCCCCCCTGGGAAAACACTCACACACACTACAAGAGGTGAAGATGACAGTGCAGGAAGATCGAAAGTGCGAATCTGACTTACGCCATTATTACGACAGTACCATTGAGTTGTGCGTGGGGGACCCAGAGATTAAAAAGACTTCCTTTAAGGGGGACTCTGGAGGCCCTCTTGTGTGTAACAAGGTGGCCCAGGGCATTGTCTCCTATGGACGAAACAATGGCATGCCTCCACGAGCCTGCACCAAAGTCTCAAGCTTTGTACACTGGATAAAGAAAACCATGAAACGCTACTAACTACAGGAAGCAAACTAAGCCCCCGCTGTAATGAAACACCTTCTCTGGAGCCAAGTCCAGATTTACACTGGGAGAGGTGCCAGCAACTGAATAAATACCTCTTAGCTGAGTGGAAAAAAAAAAAAAAAAAA

SEQ ID NO: 9 - protein, 376 aa, Homo sapiens
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 10 - DNA, 4159 bp, Homo Sapiens
gcgggagtccgcggcgagcgcagcagcagggccgggtcctgcgcctcgggggtcggcgtccaggctcggagcgcggcacggagacggcggcagcgctggactaggtggcaggccctgcatcatggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccataaagggtgcacttaccgtgcactcggccatttccctcttcctgtgtccccagatccccactacagctccaagaggatgggcctagaaagccaagtgcaaagatgagggcagattctttacctgtctgccctcatgatttgccagcatgaattcatgatgctccacactcgcttatgctacttaatcagaatcttgagaaaatagaccataatgattccctgttgtattaaaattgcagtccaaatcccataggatggcaagcaaagttcttctagaattccacatgcaattcactctggcgaccctgtgctttcctgacactgcgaatacattccttaacccgctgcctcagtggtaataaatggtgctagatattgctactattttatagatttcctggtgcttagccttataaaaaaggttgtaaaatgtacatttatattttatcttttttttttttttttttctgagacgcagtctggctctctgtcgcccaggctggagtgcagtggctcgatctcggctcactgcaagctccgcctcccgggttcacgccattctcctgcctcagcctcccgagtagctgggactacaggcgcccgccaccacgcccggctaattttttgtatttttagtagagacggggtttcaccgtgttagccaggatggtgtcgatctcctgacctcgtgatccacccgcctcggcctcccaaagtgctgggattacaggcttgagccaccgcgcccggctatattttatcttttatctttttctttgacatttaccaatcaccaagcatgcaccaaacactgctttaggcactggggacacaaaggggacagagccatcctcctttgacacctggtcttcagttctgtgcccaacgtatatagttttgacaatgaccaggttggactgtttaatgtctttcaacttaccacgtaatcctcttgtagggatcacatctttctttatgatattgtatttctctacctctaacagtaaaaattccattcaacccttaaagctcacttcaaattcttctttgagaagtttttcctttctccgcaaccagatgtacatatttgaactctctttgtacttggagggcacttctttcgtggtagttcttttatttttattaatctctgtatccttagatagtcctccaacaaccaaaggttgggactctgtcttacatatctgggtgcccctcatagtgcagtaataagtaagttgattatatacgagctatgtaacttatattttttaatggttggatatcactgagtttttttttttaagaatttttttattgaggtaaacttcacataacataaaattaactattttaaagtgagaagttcagtgccacttagtattgttaacaatgttgcataaccaccacctttatttaaagttccaaaaaaaatgttctcctctaaaaggaaaccccatcccattaagcagatactctccattccttccttcctccagcccccagcaaccaccaatctgctttctgtctctatggatttatctattcttgctattttatataaattgaattgtatgagaccttttgtgtctggcttctttcacttagtacaagtttttgagatttatttacatagtagcatgtatcaacacttcatttttatggccaaataaaattgtattatgtgtttatagcacaatttatttatccactcattcattgatggactttgggttgtttctgacttttggctattgggaatagtgctgctatgaatgtttgtgtacctgtatttgtttgaatgcctattttgcattctcttgggtatatatctaggagtggaactgctgggtcatatgttaattctatgtttagctttttgaggaacagacaaactgttttccacagcagttgaaccattccacattcccaccagcaatgtatgagaattccaatttctgtccacttcctcaccaacacttattattttccttttcctttttttaaaaaaaataagttatggccatcttagtgggtgtgaagtggtatctcattgtgttttttatttgcatttcctatgtaatgagctagaaactaaagtacaaactagatgggacatccagtccctttgatagataatgctgagtaaaaaatgagatgaaagacatttgtttgtttttagaacacgagtgacagtttgttaaaaagctttagaggaggaatgaaaacaaagtgaagtacacttagaaaagggccaagtggacatcttggatgtcaagtgcctagttcagtatcttttttttttttttttttttttttgagacagtgcctcactctgtcacccaggctggagtgtagtggcatgatctgggctcactgcaacctcctcctcctggattcaagcaattctcttgcttcagcctcccaagtagctgagactacaagcacccaccatcacacccagctaattttgtatttttcagtagagacggggtttcgccacattggccgtgttggtcttgaactcctggcctcaagcgatccgcctacctcagcctcccaaagtgctaggattacaggcataagccactgagcccagccctagttcagtatcttttatgtaaattacaaacatctgcaacattatgtatcatatgcagatacttattgcatttcttttattagtggtgaaagtgttctatgcatttattggctcttgaatttcctcatctatgaattgtcattcatacacctacttttctgcttcgtttttacatatgtctttgcctattaaagatattatccctctgttttatattttctctcattcttgtattgccttttaaattttgttatgatgtttcattaataaacagtgttttgttttcctctataaaaaaaaaaaaaaaa

SEQ ID NO: 11 - protein, 19 aa, Homo sapiens
GTEAAASSCFVAECCMESG

SEQ ID NO: 12 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 13 - DNA, 1629 bp, Homo sapiens
attcatgaaaatccactactccagacagacggctttggaatccaccagctacatccagctccctgaggcagagttgagaatggagagaatgttacctctcctggctctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctagagcttgccatcaagcagtggggctctcagtaaggaacttggaatgcaagctggatgcctgggtctctgggcacagcctggcccctgtgcaccgagtggccatggcatgtgtggccctgtctgcttatccttggaaggtgacagcgattccctgtgtagctctcacatgcacaggggcccatggactcttcagtctggagggtcctgggcctcctgacagcaataaataatttcgttggaaaaaaaaaaaaaaaaaaaaaaaaaaaaaaaaaaaaaaaa

SEQ ID NO: 14 - protein, 341 aa, Cowpox virus
MDIFREIASSMKGENVFISPPSISSVLTILYYGANGSTAEQLSKYVEKEADKNKDDISFKSMNKVYGRYSAVFKDSFLRKIGDNFQTVDFTDCRTVDAINKCVDIFTEGKINPLLDEPLSPDTCLLAISAVYFKAKWLMPFEKEFTSDYPFYVSPTEMVDVSMMSMYGEAFNHASVKESFGNFSIIELPYVGDTSMVVILPDNIDGLESIEQNLTDTNFKKWCDSMDAMFIDVHIPKFKVTGSYNLVDALVKLGLTEVFGSTGDYSNMCNSDVSVDAMIHKTYIDVNEEYTEAAAATCALVADCASTVTNEFCADHPFIYVIRHVDGKILFVGRYCSPTTN

SEQ ID NO: 15 - DNA, 1468 bp, Cowpox virus
tccatggaagaacgaaagtagtataaaagtaataaaacaaaaaaaagaatataaaaaatttatagccactttctttgaggactgttttcctgaaggaaatgaacctctggaattagttagatatatagaattagtatacacgctagattattctcaaactcctaattatgacagactacgtagactgtttatacaagattgaaaatatatttctttttattgagtggtggtagttacggatatctaatattaatattagactatctctatcgtcacacaacaaaatcgattgccatggatatcttcagggaaatcgcatcttctatgaaaggagagaatgtattcatttctccaccgtcaatctcgtcagtattgacaatactgtattatggagctaatggatccactgctgaacagctatcaaaatatgtagaaaaggaggcggacaagaataaggatgatatctcattcaagtccatgaataaagtatatgggcgatattctgcagtgtttaaagattcctttttgagaaaaattggagataatttccaaactgttgacttcactgattgtcgcactgtagatgcgatcaacaagtgtgttgatatcttcactgaggggaaaattaatccactattggatgaaccattgtctccagatacctgtctcctagcaattagtgccgtatactttaaagcaaaatggttgatgccatttgaaaaggaatttaccagtgattatcccttttacgtatctccaacggaaatggtagatgtaagtatgatgtctatgtacggcgaggcatttaatcacgcatctgtaaaagaatcattcggcaacttttcaatcatagaactgccatatgttggagatactagtatggtggtaattcttccagacaatattgatggactagaatccatagaacaaaatctaacagatacaaattttaagaaatggtgtgactctatggatgctatgtttatcgatgtgcacattcccaagtttaaggtaacaggctcgtataatctggtggatgcgctagtaaagttgggactgacagaggtgttcggttcaactggagattatagcaatatgtgtaattcagatgtgagtgtcgacgctatgatccacaaaacgtatatagatgtcaatgaagagtatacagaagcagctgcagcaacttgtgcgctggtggcagactgtgcatcaacagttacaaatgagttctgtgcagatcatccgttcatctatgtgattaggcatgtcgatggcaaaattcttttcgttggtagatattgctctccaacaactaattaaatcacattcttaatattagaatattagaatattatatagttaagatttttactaattggttaaccatttttttaaaaaaatagaaaaaaaacatgttatattagcgagggtcgttattcttccaattgcaattggtaagatgacggcc

SEQ ID NO: 16 - protein, 65 aa, Homo sapiens
MVAKQRIRMANEKHSKNITQRGNVAKTLRPQEEKYPVGPWLLALFVFVVCGSAIFQIIQSIRMGM

SEQ ID NO: 17 - DNA, 842 bp, Homo sapiens
gcctctctctggagtcggctagccggggctcggggagcggggtgcgcagggctcggggccacgccttgccacctgcagcgcccgggtgggccgcgggggcctcggcgggacgcgctcggccctgtcgcaggagctaacgcagggggaatccttgcaggtgggagcatttcagagcgcacaagccatggtggccaaacagcggatccggatggctaacgagaagcacagcaaaaacatcacccagagggggaacgtagccaaaaccctgaggccgcaagaggagaaatatcctgtgggaccatggctgttggcactgtttgtttttgttgtctgtggctcagctatctttcagatcattcagagcataaggatgggcatgtgagaaagccagggatttgacaccacctccctcccactggaggcgggaggacaacggaagcggtcagccagtttctgcgggaaacaagcaggccacacggaatagaaaaaaacgctcccccacttgttccctgatcacttcatcgtggatgtcagaccaaattgccttctcacaggacatcttggtgcatccgcgttctcaagcggaaaggacattttgcttttctgttggcaggattagtagccacgcgggtcgtccgcagcagtgctgtctttttggtttttcccttggtttcactaatgcgtgcatgtggccctctgaacgatcactggtttactttctatggatacaatctctcctccattgagaattgattttacaaataaatgtcttcgttcaaccttaaaaaaaaaaaaaaaaaaaaaaaaaaaaaaaaaaaaaaaaaaaaaaaaaaaaaaaaaaa

SEQ ID NO: 18 - protein, 37 aa, Homo sapiens
VEVNEEGTEAAAASSCFVVAECCMESGPRFCADHPFL

SEQ ID NO: 19 - protein, 37 aa, Artificial Sequence
  Synthetic
VEVNEEGTEAAAASSCFVVADCCMESGPRFCADHPFL

SEQ ID NO: 20 - protein, 37 aa, Artificial Sequence
  Synthetic
VEVNEEGTEAAAASSCFVVAACCMESGPRFCADHPFL

SEQ ID NO: 21 - protein, 37 aa, Artificial Sequence
  Synthetic
VEVNEEGREAAAASSCFVVAECCMESGPRFCADHPFL

SEQ ID NO: 22 - protein, 37 aa, Cowpox virus
IDVNEEYTEAAAATCALVADCASTVTNEFCADHPFIP

(19) United States
(12) Patent Application Publication
(10) Pub. No.: US 2014/0056964 A1
(43) Pub. Date: Feb. 27, 2014

(54) GRANZYME B INHIBITOR COMPOSITIONS, METHODS AND USES FOR PROMOTING WOUND HEALING

(71) Applicants: Paul R. Hiebert, Vancouver (CA); Darryl A. Knight, Vancouver (CA); David J. Granville, Port Coquitlam (CA); Wendy A. Boivin, Coquitlam (CA); Dawn M. Cooper, Vancouver (CA)

(72) Inventors: Paul R. Hiebert, Vancouver (CA); Darryl A. Knight, Vancouver (CA); David J. Granville, Port Coquitlam (CA); Wendy A. Boivin, Coquitlam (CA); Dawn M. Cooper, Vancouver (CA)

(73) Assignee: THE UNIVERSITY OF BRITISH COLUMBIA, Vancouver, BC (CA)

(21) Appl. No.: 13/992,139

(22) PCT Filed: Dec. 6, 2011
(86) PCT No.: PCT/IB11/03207

§ 371 Date: Oct. 28, 2013

Related U.S. Application Data
(60) Provisional application No. 61/420,230, filed on Dec. 6, 2010.
(60) Provisional application No. 61/493,265, filed on Jun. 3, 2011.

(51) Int. Cl. A61K 38/00; C07K 5/117; C07K 5/103; C07D 487/04; A61K 45/00; C07D 307/33; A61K 38/06; A61K 38/07; A61K 31/55; A61K 31/365; C07K 5/097; C07D 307/83

(52) U.S. Cl. CPC A61K 38/005; C07K 5/0823; C07K 5/1024; C07K 5/1005; C07D 487/04; C07D 307/83; C07D 307/33; A61K 38/06; A61K 38/07; A61K 31/55; A61K 31/365; A61K 45/05
USPC 424/446; 514/20.3; 514/214.01; 514/470; 514/473; 514/18.6; 514/17.2; 424/158.1; 514/44 A; 424/146.1; 435/184

(57) ABSTRACT

Methods of promoting wound healing in a subject is disclosed. The method include applying a Granzyme B (Granzyme B) inhibitor to the wound. The wound may be a skin wound. The Granzyme B inhibitor may be comprised of nucleic acids, or peptides, including but not limited to antibodies, or small molecules.

### RELATED APPLICATIONS

This application claims priority to U.S. Provisional Application No. 61/420,230 filed on Dec. 6, 2010 and U.S. Provisional Application No. 61/493,265 filed on Jun. 3, 2011, the entire contents of which are incorporated herein by this reference.

### FIELD OF THE INVENTION

The invention relates to compositions, methods, and uses for wound healing.

### BACKGROUND OF THE INVENTION

Wound healing is an intricate process in which an organ, such as the skin, is repaired after injury. In normal skin, the epidermis and dermis form a protective barrier against the external environment. Once this protective barrier is broken, wound healing is set in motion to once again repair the protective barrier.

The protective barrier can be weakened and/or ultimately broken by environmental factors such as exposure to UV light, chemical, heat or mechanical injury to the skin. Additionally, biologic and genetic factors can play a pan in weakening or breaking the protective barrier. For example, diseases such as diabetes and psoriasis can disrupt the protective barrier. Further, natural conditions such as biological and/or environmentally-induced aging can result in disruption or thinning of the skin's protective barrier. Immobilization or obsesity may also lead to disruption or thinning of the skin's protective barrier. All of these conditions can lead to skin tearing or ulceration caused by pressure, ischemia, friction, chemical, heat, or other trauma to the skin (see, for e.g., Sen et al., 2009). In many cases these wounds may not heal completely or properly due to these underlying conditions.

Given the high costs for health care of subjects having chronic wounds, or wounds that fail to close properly or recur, approximately $25 billion annually, there is a need in the art for the identification of compounds, compositions, and methods to promote wound healing and/or prevent the occurrence or re-occurrence of such wounds.

### SUMMARY

In some embodiments, the present invention is based, at least in part, on the discovery that Granzyme B cleaves the extracellular matrix proteins, decorin, biglycan, betaglycan, syndecan, brevican, fibromodulin, fibrillin-1, fibrillin-2, and fibulin-2 in vitro and cleavage of decorin, biglycan, betaglycan by Granzyme B is concentration-dependent. Cleavage of decorin, biglycan, and betaglycan by Granzyme B releases active TGF-β. The release of TGF-β was specific to cleavage of decorin, biglycan, and betaglycan by Granzyme B as TGF-β was not released in the absence of Granzyme B or when Granzyme B was inhibited by DCI. In addition, it has been shown that Granzyme B cleaves the proteoglycan substrates, biglycan and betaglycan at a P1 residue of Asp (biglycan: D⁹¹, betaglycan: D⁵⁵⁸).

In some embodiments, the present invention is further based, at least in part, on the discovery that, in vivo, deletion of Granzyme B delays the onset of skin frailty, hair loss, hair graying and the formation of inflammatory subcutaneous skin lesions or xanthomas in the ApoE knockout mouse. It has also been shown that Granzyme B is expressed in areas of collagen and decorin degradation and remodelling in the skin of apoE-KO mice and that Granzyme B deficiency protects against skin thinning due, at least in part, to inhibition of decorin cleavage and/or an increase in dermal thickness.

Furthermore, the present invention demonstrates that inhibitors of Granzyme B downmodulate decorin cleavage in vitro and in vivo and promote wound healing by, for example, stimulating collagen organization, decreasing scarring and increasing the tensile strength of skin.

Accordingly, in one aspect, there is provided a method of promoting wound healing in a subject. The method involves applying a Granzyme B (Granzyme B) inhibitor to the wound. The wound may be, without limitation, a skin wound.

The Granzyme B inhibitor may be selected from one or more of the following: nucleic acids, peptides, and small molecules. Optionally, the peptide may be an antibody. Optionally, the antibody may be a monoclonal antibody.

The Granzyme B inhibitor may be selected from one or more of the following: Azepino[3,2,1-hi]indole-2-carboxamide, 5-[[(2S,3S)-2-[(2-benzo[b]thien-3-ylacetyl)amino]-3-methyl-1-oxopentyl]amino]-1,2,4,5,6,7-hexahydro-4-oxo-N-(1H-1,2,3-triazol-5-ylmethyl)-, (2S,5S)-(compound 20 from Willoughby et al. (2002) Bioorganic & Medicinal Chemistry Letters 12:2197-2200) referred to herein as Willoughby 20 and different batches of Willoughby 20 are referred to herein as JT25102B and JT00025135; Bio-x-IEPD^{P}-(OPh)₂; (2S,5S)-5-[(N-acetyl-L-isoleucyl)amino]-4-oxo-N-(1H-tetraazol-5-ylmethyl)-1,2,4,5,6,7-hexahydroazepino[3,2,1-hi]indole-2-carboxamide; (2S,5S)-5-[(N-acetyl-L-isoleucyl)amino]-4-oxo-N-(1H-1,2,3-triazol-4-ylmethyl)-1,2,4,5,6,7-hexahydroazepino[3,2,1-hi]indole-2-carboxamide; (2S,5S)-5-([(2R)-3-methyl-2-pyridin-2-ylbutanoyl]amino)-4-oxo-N-(1H-1,2,3-triazol-4-ylmethyl)-1,2,4,5,6,7-hexahydroazepino[3,2,1-hi]indole-2-carboxamide; (2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino) propanoylamino]propanoylamino]pentanoic acid; 5-chloro-4-oxo-2-[2-[2-(phenylmethoxycarbonylamino) propanoylamino]propanoylamino]pentanoic acid; (4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid; (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid, protease inhibitor-9 or derivatives thereof, CrmA, serp-2, ZINC05723764, ZINC05723787, ZINC05316154, ZINC05723499, ZINC05723646, ZINC05398428, ZINC05723503, ZINC05723446, ZINC05317216, ZINC05315460, ZINC05316859, and ZINC05605947. Alternatively, the Granzyme B inhibitor may be selected from one or more of the following: Willoughby 20, NCI 644752, NCI 644777, ZINC05317216, and NCI 630295.

Optionally, the Granzyme B inhibitor may be formulated for topical administration. The Granzyme B inhibitor may be formulated for co-administration with another wound treatment. Another wound treatment may be selected from one or more of the following: a topical antimicrobial; a cleanser; a wound gel; a collagen; an elastin; a tissue growth promoter; an enzymatic debriding preparation; an antifungal; an anti-inflammatory; a barrier; a moisturizer; and a sealant. Optionally, the another wound treatment may be selected from one or more of the following: a wound covering, a wound filler, and an implant. Optionally, the another wound treatment may be selected from one or more of the following: absorptive dressings; alginate dressings; foam dressings; hydrocolloid dressings; hydrofiber dressings; compression dressing and wraps; composite dressing; contact layer; wound gel impregnated gauzes; wound gel sheets; transparent films; wound fillers; dermal matrix products or tissue scaffolds; and closure devices. Optionally, the Granzyme B inhibitor may be formulated for topical application in a wound covering, a wound filler, or an implant. Optionally, the Granzyme B inhibitor may be formulated for impregnation in a wound covering, a wound filler or an implant. The subject may be a mammal; optionally, the subject may be a human.

In another aspect, use of a Granzyme B inhibitor to promote wound healing in a subject is disclosed. In another aspect, use of a Granzyme B inhibitor in the preparation of a medicament for promoting wound healing in a subject is disclosed. Optionally, the wound may be a skin wound. Optionally, the Granzyme B inhibitor may be selected from one or more of the following: nucleic acids, peptides and small molecules. Optionally, the peptides may be antibodies. Optionally, the antibodies may be monoclonal antibodies.

Optionally, the Granzyme B inhibitor used herein may be selected from one or more of the following: Azepino[3,2,1-hi]indole-2-carboxamide, 5-[[(2S,3S)-2-[(2-benzo[b]thien-3-ylacetyl)amino]-3-methyl-1-oxopentyl]amino]-1,2,4,5,6,7-hexahydro-4-oxo-N-(1H-1,2,3-triazol-5-ylmethyl)-, (2S,5S)-(compound 20 from Willoughby et al. (2002) Bioorganic & Medicinal Chemistry Letters 12:2197-2200) referred to herein as Willoughby 20; Bio-x-IEPD^{P}-(OPh)₂; (2S,5S)-5-[(N-acetyl-L-isoleucyl)amino]-4-oxo-N-(1H-tetraazol-5-ylmethyl)-1,2,4,5,6,7-hexahydroazepino[3,2,1-hi]indole-2-carboxamide; (2S,5S)-5-[(N-acetyl-L-isoleucyl)amino]-4-oxo-N-(1H-1,2,3-triazol-4-ylmethyl)-1,2,4,5,6,7-hexahydroazepino[3,2,1-hi]indole-2-carboxamide; (2S,5S)-5-{[(2R)-3-methyl-2-pyridin-2-ylbutanoyl]amino}-4-oxo-N-(1H-1,2,3-triazol-4-ylmethyl)-1,2,4,5,6,7-hexahydroazepino[3,2,1-hi]indole-2-carboxamide; (2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino)propanoylamino]propanoylamino]pentanoic acid; 5-chloro-4-oxo-2-[2-[2-(phenylmethoxycarbonylamino)propanoylamino]propanoylamino]pentanoic acid; (4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid; (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid, protease inhibitor-9 or derivatives thereof, CrmA, serp-2, ZINC05723764, ZINC05723787, ZINC05316154, ZINC05723499, ZINC05723646, ZINC05398428, ZINC05723503, ZINC05723446, ZINC05317216, ZINC05315460, ZINC05316859, and ZINC05605947. Alternatively, the Granzyme B inhibitor may be selected from one or more of the following: Willoughby 20, NCI 644752, NCI 644777, ZINC05317216, and NCI 630295.

Optionally, the Granzyme B inhibitor being used is formulated for topical administration. Optionally, the Granzyme B inhibitor is formulated for co-administration with another wound treatment. Optionally, the wound treatment is selected from one or more of: a topical antimicrobial; a cleanser; a wound gel; a collagen; a elastin; a tissue growth promoter; an enzymatic debriding preparation; an antifungal; an anti-inflammatory; a barrier; a moisturizer; and a sealant. Optionally, the another wound treatment is selected from one or more of: a wound covering, a wound filler and an implant. Optionally, the another wound treatment is selected from one or more of: absorptive dressings; alginate dressings; foam dressings; hydrocolloid dressings; hydrofiber dressings; compression dressing & wraps; composite dressing; contact layer; wound gel impregnated gauzes; wound gel sheets; transparent films; wound fillers; dermal matrix products or tissue scaffolds; and closure devices. Optionally, the Granzyme B inhibitor is formulated for topical application in a wound covering, a wound filler, or an implant. Optionally, the Granzyme B inhibitor is formulated for impregnation in a wound covering, a wound filler or an implant. Optionally, the use involves a subject that may be a mammal; optionally, the use involves a subject that may be a human.

In another aspect, a Granzyme B inhibitor for use in promoting wound healing in a subject is disclosed herein. Optionally, the wound may be a skin wound. Optionally, the Granzyme B inhibitor may be selected from one or more of the following: nucleic acids, peptides, and small molecules. Optionally, the peptides may be antibodies. Optionally, the antibodies may be monoclonal antibodies. Optionally, the Granzyme B inhibitor may be selected from one or more of the following: Azepino[3,2,1-hi]indole-2-carboxamide, 5-[[(2S,3S)-2-[(2-benzo[b]thien-3-ylacetyl)amino]-3-methyl-1-oxopentyl]amino]-1,2,4,5,6,7-hexahydro-4-oxo-N-(1H-1,2,3-triazol-5-ylmethyl)-, (2S,5S)-(compound 28 from Willoughby et al. (2002) Bioorganic & Medicinal Chemistry Letters 12:2197-2200) referred to herein as Willoughby 20; Bio-x-IEPD^{P}-(OPh)₂; (2S,5S)-5-[(N-acetyl-L-isoleucyl)amino]-4-oxo-N-(1H-tetraazol-5-ylmethyl)-1,2,4,5,6,7-hexahydroazepino[3,2,1-hi]indole-2-carboxamide; (2S,5S)-5-[(N-acetyl-L-isoleucyl)amino]-4-oxo-N-(1H-1,2,3-triazol-4-ylmethyl)-1,2,4,5,6,7-hexahydroazepino[3,2,1-hi]indole-2-carboxamide; (2S,5S)-5-{[(2R)-3-methyl-2-pyridin-2-ylbutanoyl]amino}-4-oxo-N-(1H-1,2,3-triazol-4-ylmethyl)-1,2,4,5,6,7-hexahydroazepino[3,2,1-hi]indole-2-carboxamide; (2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino)propanoylamino]propanoylamino]pentanoic acid; 5-chloro-4-oxo-2-[2-[2-(phenylmethoxycarbonylamino)propanoylamino]propanoylamino]pentanoic acid; (4S)-4-[[(2S)-2-acetamido-4-methylpentanoyl]amino]-5-[2-[(2S)-4-hydroxy-1,4-dioxobutan-2-yl]carbamoyl]pyrrolidin-1-yl]-5-oxopentanoic acid; (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid, protease inhibitor-9 or derivatives thereof, CrmA, serp-2, ZINC05723764, ZINC05723787, ZINC05316154, ZINC05723499, ZINC05723646, ZINC05398428, ZINC05723503, ZINC05723446, ZINC05317216, ZINC05315460, ZINC05316859, and ZINC05605947. Alternatively, the Granzyme B inhibitor may be selected from one or more of the following: Willoughby 20, NCI 644752, NCI 644777, ZINC05317216, and NCI 630295.

Optionally, the Granzyme B inhibitor may be formulated for topical administration. Optionally, the Granzyme B inhibitor may be formulated for co-administration with another wound treatment. Optionally, the another wound treatment may be selected from one or more of: a topical antimicrobial; a cleanser; a wound gel; a collagen; an elastin; a tissue growth promoter; an enzymatic debriding preparation; an antifungal; an anti-inflammatory; a barrier; a moisturizer; and a sealant. Optionally, the another wound treatment may be selected from one or more of: a wound covering, a wound filler and an implant. Optionally, the another wound treatment may be selected from one or more of: absorptive dressings; alginate dressings; foam dressings; hydrocolloid dressings; hydrofiber dressings; compression dressing & wraps; composite dressing; contact layer; wound gel impregnated gauzes; wound gel sheets; transparent films; wound fillers; dermal matrix products or tissue scaffolds; and closure devices. Optionally, the Granzyme B inhibitor may be formulated for topical application in a wound covering, a wound filler, or an implant. Optionally, the Granzyme B inhibitor may be formulated for impregnation in a wound covering, a wound filler or an implant. Optionally, the subject may be a mammal; optionally the subject may be a human.

In another aspect, a method of inhibiting release of a cytokine, such as active transforming growth factor-β (TGF-β), wherein the cytokine, e.g. TGF-, is bound to an extracellular matrix protein, e.g., an extracellular proteoglycan, is disclosed. The method may involve inhibiting a cleavage site in a proteoglycan. The proteoglycan may be selected from any one of the following: biglycan, decorin, finromodulin, or betaglycan. However, the aforementioned examples are provided as examples only and are not present as limitations. While the method disclosed details TGF-β bound to a proteoglycan, other cytokines and growth factors bound to other proteoglycans may also be considered as suitable targets. Optionally, the method is carried out in vitro. Optionally, the method is carried out in a subject in vivo. Optionally, the subject may be a mammal. Optionally, the subject may be a human. Optionally, the cleavage sites occur in any one of the following peptide sequences: Asp⁹¹Thr-Thr-Leu-Leu-Asp; or Asp⁵⁵⁸Ala-Ser-Leu-Phe-Thr; or Asp³¹Glu-Ala-Ser-Gly; or Asp⁶⁹Leu-Gly-Asp-Lys; or Asp⁸²Thr-Thr-Leu-Leu-Asp; or Asp²⁶¹Asn-Gly-Ser-Leu-Ala.

In another aspect, a model for studying age-related wound healing is disclosed. The model comprises an apolipoprotein E-knock out mouse maintained on a high-fat feed diet, wherein the high-fat feed diet is sufficient to result in xanthomatotic skin lesions on skin of the mouse. Alternatively or in addition, the high-fat feed diet may be sufficient to result in premature aging in non-xanthamatous skin. As detailed herein, inhibition of Granzyme B by way of Granzyme B inhibitors or through knock-out technology reduces the age-related loss of skin thickness, collagen density, collagen disorganization, and loss of tensile strength. It is considered that based on the results herein that a Granzyme B inhibitor could be added to Stage I skin ulcers to restore skin thickness, skin integrity, skin collagenicity, and to inhibit or otherwise reduce progression of the skin ulcer.

In another aspect, a model for studying Granzyme B protein expression in vivo is disclosed. The model comprises an apolipoprotein E-knock out mouse maintained on a high-fat feed diet, wherein the high-fat feed diet is sufficient to result in xanthomatotic skin lesions on the skin of the mouse mouse, and wherein the skin lesions express Granzyme B.

In another aspect, a model for screening compounds involved in repairing wounds is disclosed. The method involves maintaining an apolipoprotein E-knock out mouse on a high-fat feed diet, wherein the high-fat feed diet is sufficient to result in skin lesions on the mouse; administering a compound to the skin lesions on the mouse; and monitoring the skin lesions on the mouse.

In another aspect, a model for studying age-related wound healing in skin is disclosed. The model comprises an apolipoprotein E-knock-out mouse maintained on a high-fat feed diet, wherein the high-fat feed diet is sufficient to result in premature aging of the skin.

In another aspect, a method of screening compounds involved in repairing wounds is disclosed. The method may involve maintaining an apolipoprotein E-knock out mouse on a high-fat feed diet, wherein the high-fat feed diet is sufficient to result in skin lesions on the mouse, and wherein the skin lesions express Granzyme B; administering a compound to the skin lesions on the mouse; and monitoring the skin lesions on the mouse.

In another aspect, a method of screening compounds involved in inhibiting or reducing skin lesions is disclosed. The method may involve maintaining an apolipoprotein E-knock out mouse on a high-fat feed diet, wherein the high-fat feed diet is sufficient to result in skin lesions on the mouse when a compound is not administered to the mouse; administering the compound to the mouse; and monitoring the skin lesions on the mouse.

In another aspect, a method of screening compounds involved in inhibiting or reducing skin lesions is disclosed. The method may involve maintaining an apolipoprotein E-knock out mouse on a high-fat feed diet, wherein the high-fat feed diet is sufficient to result in skin lesions on the mouse when a compound is not administered to the mouse, and wherein the skin lesions express Granzyme B; administering the compound to the skin lesions on the mouse; and monitoring the skin lesions on the mouse.

In another aspect, a method of inhibiting or reducing skin tearing is disclosed. The method may involve applying a Granzyme B inhibitor to the skin. The Granzyme B inhibitor selected may be one or more of the following: nucleic acids, peptides, and small molecules. The peptides may be antibodies. The antibodies may be monoclonal antibodies. The Granzyme B inhibitor may be selected from one or more of the following: Azepino[3,2,1-hi]indole-2-carboxamide, 5-[[(2S,3S)-2-[(2-benzo[b]thien-3-ylacetyl)amino]-3-methyl-1-oxopentyl]amino]-1,2,4,5,6,7-hexahydro-4-oxo-N-(1H-1,2,3-triazol-5-ylmethyl)-, (2S,5S)-(compound 20 from Willoughby et al. (2002) Bioorganic & Medicinal Chemistry Letters 12:2197-2200) referred to herein as Willoughby 20; Bio-x-IEPD^{P}-(OPh)₂; (2S,5S)-5-[(N-acetyl-L-isoleucyl)amino]-4-oxo-N-(1H-tetraazol-5-ylmethyl)-1,2,4,5,6,7-hexahydroazepino[3,2,1-hi]indole-2-carboxamide; (2S,5S)-5-[(N-acetyl-L-isoleucyl)amino]-4-oxo-N-(1H-1,2,3-triazol-4-ylmethyl)-1,2,4,5,6,7-hexahydroazepino[3,2,1-hi]indole-2-carboxamide; (2S,5S)-5-{[(2R)-3-methyl-2-pyridin-2-ylbutanoyl]amino}-4-oxo-N-(1H-1,2,3-triazol-4-ylmethyl)-1,2,4,5,6,7-hexahydroazepino[3,2,1-hi]indole-2-carboxamide; (2S,5S)-4-oxo-5-({[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino)propanoylamino]propanoylamino]pentanoic acid; 5-chloro-4-oxo-2-[2-[2-(phenylmethoxycarbonylamino)propanoylamino]propanoylamino]pentanoic acid; (4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid; (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid, protease inhibitor-9 or derivatives thereof, CrmA, serp-2, ZINC05723764, ZINC05723787, ZINC05316154, ZINC05723499, ZINC05723646, ZINC05398428, ZINC05723503, ZINC05723446, ZINC05317216, ZINC05315460, ZINC05316859, and ZINC05605947. Alternatively, the Granzyme B inhibitor may be selected from one or more of the following: Willoughby 20, NCI 644752, NCI 644777, ZINC05317216, and NCI 630295. Further, the Granzyme B inhibitor may be formulated for topical administration.

In another aspect, the present invention provides methods of promoting wound healing in a subject, the method comprising administering a Granzyme B (GrB) inhibitor to the subject for a time and in an amount sufficient to promote would healing, thereby promoting wound healing in the subject.

In another aspect, the present invention provides methods of promoting wound healing in a subject, the method comprising applying a Granzyme B (Granzyme B) inhibitor to the wound, for a time and in an amount sufficient to promote would healing, thereby promoting wound healing in the subject.

The wound may be a chronic wound, such as a chronic skin wound, such as a pressure ulcer.

In one embodiment, cleavage of an extracellular matrix protein is inhibited. In one embodiment, the extracellular matrix protein is selected from the group consisting of decorin, biglycan, betaglycan, syndecan, brevican, fibromodulin, fibrillin-1, fibrillin-2, and fibulin-2. In one embodiment, the extracellular matrix protein is decorin.

In one embodiment, release of TGFβ bound to an extracellular matrix protein is inhibited. In one embodiment, the extracellular matrix protein is decorin.

In another aspect, the present invention provides methods of preventing skin tearing of a subject, comprising applying a Granzyme B inhibitor to the skin of the subject for a time and in an amount sufficient to prevent skin tearing, thereby preventing skin tearing in the subject.

In one embodiment, the skin tearing is associated with a chronic wound. In another embodiment, the skin tearing is associated with aging.

In one embodiment, cleavage of an extracellular matrix protein is inhibited. In one embodiment, extracellular matrix protein is selected from the group consisting of decorin, biglycan, betaglycan, syndecan, brevican, fibromodulin, fibrillin-1, fibrillin-2, and fibulin-2. In one embodiment, the extracellular matrix protein is decorin.

In one embodiment, release of TGFβ bound to an extracellular matrix protein is inhibited. In one embodiment, the extracellular matrix protein is decorin.

In yet another aspect, the present invention provides methods for inhibiting hypertrophic scarring of a wound, comprising applying a Granzyme B inhibitor to the skin of the subject for a time and in an amount sufficient to prevent skin hypertrophic scarring of a wound, thereby inhibiting hypertrophic scarring of a wound.

In one embodiment, cleavage of an extracellular matrix protein is inhibited. In one embodiment, the extracellular matrix protein is selected from the group consisting of decorin, biglycan, betaglycan, syndecan, brevican, fibromodulin, fibrillin-1, fibrillin-2, and fibulin-2. In one embodiment, the extracellular matrix protein is decorin.

In one embodiment, release of TGFβ bound to an extracellular matrix protein is inhibited. In one embodiment, the extracellular matrix protein is decorin.

In another aspect, the present invention provides methods for increasing collagen organization in the skin of a subject, comprising applying a Granzyme B inhibitor to the skin of the subject in an amount and for a time sufficient to increase collagen organization in the subject, thereby increasing collagen organization in the skin of the subject.

In one embodiment, cleavage of an extracellular matrix protein is inhibited. In one embodiment, the extracellular matrix protein is selected from the group consisting of decorin, biglycan, betaglycan, syndecan, brevican, fibromodulin, fibrillin-1, fibrillin-2, and fibulin-2. In one embodiment, the extracellular matrix protein is decorin

In one embodiment, release of TGFβ bound to an extracellular matrix protein is inhibited. In one embodiment, the extracellular matrix protein is decorin.

In another aspect, the present invention provides methods for increasing the tensile strength of a healing or healed skin wound of a subject, comprising applying a Granzyme B inhibitor to the skin of the subject in an amount and for a time sufficient to increase increase the tensile strength of the healing or healed skin wound of the subject, thereby increasing the tensile strength of a healing or healed skin wound of a subject.

In one embodiment, cleavage of an extracellular matrix protein is inhibited. In one embodiment, the extracellular matrix protein is selected from the group consisting of decorin, biglycan, betaglycan, syndecan, brevican, fibromodulin, fibrillin-1, fibrillin-2, and fibulin-2. In one embodiment, the extracellular matrix protein is decorin

In one embodiment, release of TGFβ bound to an extracellular matrix protein is inhibited. In one embodiment, the extracellular matrix protein is decorin.

In one aspect, the present invention provides methods for inhibiting release of TGFβ bound to an extracellular protein, comprising contacting the extracellular proteoglycan with a Granzyme B inhibitor, thereby inhibiting release of TGFβ bound to the extracellular protein.

In one embodiment, the protein is selected from the group consisting of decorin, biglycan, betaglycan, syndecan, brevican, fibromodulin, fibrillin-1, fibrillin-2, and fibulin-2. In one embodiment, the protein is decorin

In another aspect, the present invention provides methods inhibiting extracellular decorin cleavage, comprising contacting decorin with a Granzyme B inhibitor, thereby inhibiting extracellular decorin cleavage.

In one embodiment, the Granzyme B inhibitor for use in any of the foregoing methods is selected from the group consisting of a nucleic acid molecule, a peptide, an antibody, and a small molecule. In one embodiment, the antibody is a monoclonal antibody.

In another embodiment, the Granzyme B inhibitor for use in any of the foregoing methods is wherein the Granzyme B inhibitor is selected from one or more of the following:
2S,5S)—N-((2H-tetrazol-5-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((R)-3-methyl-2-(pyridin-2-yl)butanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide;
(2S,5S)—N-((1H-1,2,4-triazol-3-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-pyrazol-3-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-pyrazol-4-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-imidazol-4-yl)methyl)-S-((2S,3S)-2-acetamido-3-methylpentanamido)-4-oxo-1,2,4,5,6,7-hexahydroazepino[3,2,1-hi]indole-2-carboxamide;
2S,5S)-5-((2S,3S)-2-acetamido-3-methylpentanamido)-4-oxo-N-(thiazol-5-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(isoxazol-3-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-4-oxo-N-(thiazol-2-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(isoxazol-5-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-4-oxo-N-(thiazol-4-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyrimidin-5-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridazin-4-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-2-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-3-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-4-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(imidazo[1,2-a]pyrimidin-2-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-N-((3a,7a-dihydrobenzo[d]thiazol-2-yl)methyl)-4-oxo-1,2,4,5,6,7-hexahydroazepino[3,2,1-hi]indole-2-carboxamide;
(2S,5S)—N-((2H-tetrazol-5-yl)methyl)-5-((R)-3-methyl-2-(pyridin-2-yl)butanamido)-4-oxo-1,2,4,5,6,7-hexahydroazepino[3,2,1-hi]indole-2-carboxamide;
(2S,5S)—N-((2H-tetrazol-5-yl)methyl)-5-((S)-3-methyl-2-(pyridin-2-yl)butanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(2,3-difluorophenyl)acetamido)-3-methylpentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide;
(R)—N-((2S,5S)-2-((1H-1,2,3-triazol-4-yl)methylcarbamoyl)-4-oxo-1,2,4,5,6,7-hexahydroazepino[3,2,1-hi]indol-5-yl)-3-acetyl-5,5-dimethylthiazolidine-4-carboxamide;
(2S,5S)—N-((1H-1,2,3-triazol-4-yl)methyl)-5-((2S,3S)-3-methyl-2-(2-oxopyrrolidin-1-yl)pentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-(2-cyclopentylacetamido)-4-oxo-1,2,4,5,6,7-hexahydroazepino[3,2,1-hi]indole-2-carboxamide;
(2S,5S)—N-((1H-1,2,3-triazol-4-yl)methyl)-5-((S)-2-acetamido-2-cyclopropylacetamido)-4-oxo-1,2,4,5,6,7-hexahydroazepino[3,2,1-hi]indole-2-carboxamide;
(2S,5S)—N-((1H-1,2,3-triazol-4-yl)methyl)-5-((S)-2-acetamido-2-cyclopentylacetamido)-4-oxo-1,2,4,5,6,7-hexahydroazepino[3,2,1-hi]indole-2-carboxamide;
Bio-x-IEPD^{P}-(OPh)₂;
azepino[3,2,1-hi]indole-2-carboxamide;
(4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
(4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid;
5-chloro-4-oxo-3-[2-[2-(phenylmethoxycarbonylamino)propanoylamino]propanoylamino]pentanoic acid;
5-chloro-4-oxo-2-[2-[2-(phenylmethoxycarbonylamino)propanoylamino]propanoylamino]pentanoic acid;
ZINC05723764;
ZINC05723787;
ZINC05316154;
ZINC05723499;
ZINC05723646;
ZINC05398428;
ZINC05723503;
ZINC05723446;
ZINC05317216;
ZINC05315460;
ZINC05316859;
ZINC05605947;
an isocoumarin;
a peptide chloromethyl ketone;
a peptide phosphonate;
a Granzyme B inhibitory nucleic acid molecule;
an anti-Granzyme B antibody;
an inhibitory Granzyme B peptide;
a SerpB9 polypeptide, or fragment thereof;
5-chloro-4-oxo-2-[2-[2-(phenylmethoxycarbonylamino) propanoylamino]propanoylamino]pentanoic acid;
Ac-IEPD-CHO;
(4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
Ac-IETD-CHO;
(4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid;
(2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino) propanoylamino]propanoylamino]pentanoic acid;
5-chloro-4-oxo-2-[2-[2-(phenylmethoxycarbonylamino)propanoylamino]propanoylamino]pentanoic acid;
(4S)-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
(4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid,
a Serp2 polypeptide, or fragment thereof;
a CrmA polypeptide or fragment thereof, and
a SerpinA3 polypeptide or fragment thereof.

In one embodiment, the Granzyme B inhibitor for use in any of the foregoing methods is formulated for topical administration. In one embodiment, the Granzyme B inhibitor is formulated for co-administration with another wound treatment.

In one embodiment, the another wound treatment is selected from one or more of: a topical antimicrobial; a cleanser; a wound gel; a collagen; an elastin; a tissue growth promoter; an enzymatic debriding preparation; an antifungal; an anti-inflammatory; a barrier; a moisturizer; and a sealant. In another embodiment, the another wound treatment is selected from one or more of: a wound covering, a wound filler, and an implant. In another embodiment, another wound treatment is selected from one or more of: absorptive dressings; alginate dressings; foam dressings; hydrocolloid dressings; hydrofiber dressings; compression dressing and wraps; composite dressing; contact layer; wound gel impregnated gauzes; wound gel sheets; transparent films; wound fillers; dermal matrix products or tissue scaffolds; and closure devices.

In one embodiment, the subject is a mammal. In one embodiment, the subject is a human.

In another aspect, the present invention provides uses of a Granzyme B inhibitor as described herein to promote wound healing in a subject.

In yet another aspect, the present invention provides uses of a Granzyme B inhibitor as described herein in the preparation of a medicament for promoting wound healing in a subject.

In one embodiment, the wound is a skin wound. In one embodiment, the skin wound is a chronic skin wound.

In one embodiment, the Granzyme B inhibitor is selected from the group consisting of a nucleic acid molecule, a peptide, an antibody, and a small molecule. In one embodiment, a Granzyme B inhibitor is selected from the group consisting of
2S,5S)—N-((2H-tetrazol-5-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((R)-3-methyl-2-(pyridin-2-yl)butanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide;
(2S,5S)—N-((1H-1,2,4-triazol-3-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-pyrazol-3-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-pyrazol-4-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-imidazol-4-yl)methyl)-5-((2S,3S)-2-acetamido-3-methylpentanamido)-4-oxo-1,2,4,5,6,7-hexahydroazepino[3,2,1-hi]indole-2-carboxamide;
2S,5S)-5-((2S,3S)-2-acetamido-3-methylpentanamido)-4-oxo-N-(thiazol-5-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(isoxazol-3-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-4-oxo-N-(thiazol-2-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(isoxazol-5-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-4-oxo-N-(thiazol-4-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyrimidin-5-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridazin-4-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-2-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-3-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-4-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(imidazo[1,2-a]pyrimidin-2-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-N-((3a,7a-dihydrobenzo[d]thiazol-2-yl)methyl)-4-oxo-1,2,4,5,6,7-hexahydroazepino[3,2,1-hi]indole-2-carboxamide;
(2S,5S)—N-((2H-tetrazol-5-yl)methyl)-5-((R)-3-methyl-2-(pyridin-2-yl)butanamido)-4-oxo-1,2,4,5,6,7-hexahydroazepino[3,2,1-hi]indole-2-carboxamide;
(2S,5S)—N-((2H-tetrazol-5-yl)methyl)-5-((S)-3-methyl-2-(pyridin-2-yl)butanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(2,3-difluorophenyl)acetamido)-3-methylpentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide;
(R)—N-((2S,5S)-2-((1H-1,2,3-triazol-4-yl)methylcarbamoyl)-4-oxo-1,2,4,5,6,7-hexahydroazepino[3,2,1-hi]indol-5-yl)-3-acetyl-5,5-dimethylthiazolidine-4-carboxamide;
(2S,5S)—N-((1H-1,2,3-triazol-4-yl)methyl)-5-((2S,3S)-3-methyl-2-(2-oxopyrrolidin-1-yl)pentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-(2-cyclopentylacetamido)-4-oxo-1,2,4,5,6,7-hexahydroazepino[3,2,1-hi]indole-2-carboxamide;
(2S,5S)—N-((1H-1,2,3-triazol-4-yl)methyl)-5-((S)-2-acetamido-2-cyclopropylacetamido)-4-oxo-1,2,4,5,6,7-hexahydroazepino[3,2,1-hi]indole-2-carboxamide;
(2S,5S)—N-((1H-1,2,3-triazol-4-yl)methyl)-5-((S)-2-acetamido-2-cyclopentylacetamido)-4-oxo-1,2,4,5,6,7-hexahydroazepino[3,2,1-hi]indole-2-carboxamide;
Bio-x-IEPD^{P}-(OPh)₂;
azepino[3,2,1-hi]indole-2-carboxamide;
(4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
(4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid;
5-chloro-4-oxo-3-[2-[2-(phenylmethoxycarbonylamino) propanoylamino]propanoylamino]pentanoic acid;
5-chloro-4-oxo-2-[2-[2-(phenylmethoxycarbonylamino) propanoylamino]propanoylamino]pentanoic acid;
ZINC05723764;
ZINC05723787;
ZINC05316154;
ZINC05723499;
ZINC05723646;
ZINC05398428;
ZINC05723503;
ZINC05723446;
ZINC05317216;
ZINC05315460;
ZINC05316859;
ZINC05605947;
an isocoumarin;
a peptide chloromethyl ketone;
a peptide phosphonate;
a Granzyme B inhibitory nucleic acid molecule;
an anti-Granzyme B antibody;
an inhibitory Granzyme B peptide;
a SerpB9 polypeptide, or fragment thereof;
5-chloro-4-oxo-2-[2-[2-(phenylmethoxycarbonylamino) propanoylamino]propanoylamino]pentanoic acid;
Ac-IEPD-CHO;
(4S)-4-[[(2S)-2-acetamido-4-methylpentanoyl]amino]-5-[2-[((2S)-4-hydroxy-[4-dioxobutan-2-yl]carbamoyl]pyrrolidin-1-yl]-5-oxopentanoic acid;
Ac-IETD-CHO;
(4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid;
(2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino) propanoylamino]propanoylamino]pentanoic acid;
5-chloro-4-oxo-2-[2-[2-(phenylmethoxycarbonylamino) propanoylamino]propanoylamino]pentanoic acid;
(4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
(4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid,
a Serp2 polypeptide, or fragment thereof;
a CrmA polypeptide or fragment thereof; and
a SerpinA3 polypeptide or fragment thereof.

In one embodiment, the Granzyme B inhibitor is formulated for topical administration. In one embodiment, the Granzyme B inhibitor is formulated for co administration with another wound treatment. In one embodiment, the another wound treatment is selected from one or more of: a topical antimicrobial; a cleanser; a wound gel; a collagen; a elastin; a tissue growth promoter; an enzymatic debriding preparation; an antifungal; an anti-inflammatory; a barrier; a moisturizer; and a sealant.

In one embodiment, the subject is a mammal. In one embodiment, the subject is a human.

The present invention further provides a Granzyme B inhibitor for use in promoting wound healing in a subject. In one embodiment, the wound is a skin wound. In one embodiment, the wound is a chronic skin wound.

In one embodiment, the Granzyme B inhibitor is selected from the group consisting of a nucleic acid molecule, a peptide, and antibody, and a small molecule.

In one embodiment, the Granzyme B inhibitor is formulated for topical administration. In one embodiment, the Granzyme B inhibitor is formulated for co-administration with another wound treatment as described herein.

### BRIEF DESCRIPTION OF THE DRAWINGS

FIG. 1 demonstrates the identification of extracellular Granzyme B substrates. Star denotes full length and arrows indicate cleavage fragments.

FIG. 2 demonstrates that Granzyme B mediates cleavage of native smooth muscle cell derived decorin and biglycan.

FIGS. 3A-3C demonstrate dose dependent Granzyme B-mediated cleavage of decorin, biglycan and betaglycan.

FIG. 4 demonstrates that Granzyme B-mediated cleavage of PGs is inhibited by DCI at 4 h and 24 h and Granzyme B cleavage sites contain aspartic acid at the P1 residue.

FIG. 5 demonstrates Granzyme B cleavage of decorin, biglycan and betaglycan results in the release of active TGF-β.

FIG. 6 demonstrates that TGF-β released by Granzyme B is active and induces SMAD-3 and Erk-2 phosphorylation in HCASMCs.

FIG. 7 demonstrates Granzyme B-dependent phosphorylation of SMAD-3 by TGF-β released by Granzyme B cleavage in HCASMCs.

FIG. 8 demonstrates an analysis of gross skin pathology, morbidity and frailty.

FIG. 9 demonstrates skin morphology and xanthoma development.

FIG. 10 demonstrates an analysis of skin thickness.

FIG. 11 demonstrates an analysis of collagen and elastin remodeling in diseased skin.

FIG. 12 demonstrates an analysis of Granzyme B expression near areas of decorin and collagen remodeling.

FIG. 13 demonstrates loss of dermal collagen density in apoE-KO mice rescued by knocking out Granzyme B.

FIG. 14 demonstrates Granzyme B cleaves decorin and is present in areas of decorin degradation.

FIG. 15 demonstrates that inhibition of Granzyme B using a specific small molecule inhibitor inhibits betaglycan cleavage.

FIG. 16 demonstrates that inhibition of Granzyme B using a specific small molecule inhibitor inhibits the release of proteoglycan-sequestered TGF-β.

FIG. 17 demonstrates that inhibition of Granzyme B using a specific small molecule inhibitor inhibits decorin cleavage.

FIG. 18 demonstrates that inhibition of Granzyme B (Granzyme B) using small molecule inhibitors inhibits ECM cleavage.

FIG. 19 demonstrates that inhibition of Granzyme B (Granzyme B) using a small molecule inhibitor inhibits ECM cleavage.

FIG. 20 demonstrates that inhibition of Granzyme B (Granzyme B) using NCI 644777 inhibits betaglycan cleavage.

FIG. 21A demonstrates Granzyme B (Granzyme B) cleavage of fibronectin (FN) reduces EC adhesion to FN dose dependently also shows inhibition of Granzyme B using Willoughby 20.

FIG. 21B demonstrates that inhibition of Granzyme B (Granzyme B) using Willoughby 20 inhibits fibronectin cleavage.

FIG. 22 demonstrates that GzmB cleaves plasma fibronectin (FN) in its soluable form and matrix form.

FIG. 23 demonstrates that inhibition of Granzyme B prevents decorin degradation in chronic wounds in vivo.

### DETAILED DESCRIPTION

Until recently Granzyme B (Granzyme B) was thought to act within cells to mediate cell destruction. This cytotoxic enzyme effectively kills virally infected and malignant cells. However, as described herein, it has shown that Granzyme B when present external to cells wreaks havoc on the extracellular matrix (“ECM”) in areas of chronic inflammation and wounds. As also described herein, once Granzyme B is inhibited, the destructive cascade that is launched in the exterior environment is interrupted and resultant cellular damage is halted. As traumatic injuries are the fifth leading cause of death in North America, it is essential to find effective and alternative solutions to wound care. Currently most wound care is focused on treating symptoms, but wound repair and closure is challenging if Granzyme B is still destroying the ECM proteins needed to maintain skin integrity.

Granzyme B (Granzyme B, also referred to herein at GZMB) is a pro-apoptotic serine protease found in the granules of cytotoxic lymphocytes (CTL) and natural killer (NK) cells. Granzyme B is released towards target cells, along with the pore-forming protein, perforin, resulting in its perforin-dependent internalization into the cytoplasm and subsequent induction of apoptosis (see, for e.g., Medema et al. 1997). However, during aging, inflammation and chronic disease, Granzyme B can also be expressed and secreted by other types of immune (e.g., mast cell, macrophage, neutrophil, dendritic) or non-immune (keratinocyte, chondrocyte) cells and has been to possess extracellular matrix remodeling activity (Choy et al., 2004 and Buzza et al., 2005).

### I. Methods of the Invention

In some embodiments, the present invention is based, at least in part, on the discovery that Granzyme B cleaves the extracellular matrix proteins, decorin, biglycan, betaglycan, syndecan, brevican, fibrillin-1, fibrillin-2, and fibulin-2 in vitro and cleavage of decorin, biglycan, betaglycan by Granzyme B is concentration-dependent. Cleavage of decorin, biglycan, and betaglycan by Granzyme B releases active TGF-β. The release of TGF-β is specific to cleavage of decorin, biglycan, and betaglycan by Granzyme B as TGF-β is not released in the absence of Granzyme B or when Granzyme B is inhibited by DCI.

In addition, it has been shown that Granzyme B cleaves the proteoglycan substrates, biglycan and betaglycan at a P1 residue of Asp (biglycan: D⁹¹, betaglycan: D⁵⁵⁸).

In some embodiments, the present invention is further based, at least in part, on the discovery that, in vivo, deletion of Granzyme B delays the onset of skin frailty, hair loss, hair graying and the formation of inflammatory subcutaneous skin lesions or xanthomas in the ApoE knockout mouse. It has also been shown that Granzyme B is expressed in areas of collagen and decorin degradation and remodelling in the skin of apoE-KO mice and that Granzyme B deficiency protects against skin thinning due in part to an increase in dermal thickness, an increase in collagen density, and/or an increase in collagen organization. Furthermore, the present invention demonstrates that inhibitors of Granzyme B downmodulate decorin and biglycan cleavage in vitro and in vivo and promote wound healing by, for example, stimulating collagen organization, decreasing scarring and increasing the tensile strength of skin.

Accordingly, the present invention provides, among others, methods for promoting wound healing, inhibiting release of TGFβ bound to an extracellular matric proteins, e.g., extracellular proteoglycans, methods of preventing hypertrophic scarring of a wound, and methods of preventing skin tearing.

In one aspect, the present invention provides methods for promoting wound healing in a subject having a wound. The present invention further provides use of a Granzyme B inhibitor to promote wound healing in a subject. In another aspect, use of a Granzyme B inhibitor in the preparation of a medicament for promoting wound healing in a subject is disclosed.

As used herein, the term “wound healing” also known as “cicatrisation”, is a process in which the skin (or another organ-tissue) repairs itself after injury. In normal skin, the epidermis (outermost layer) and dermis (inner or deeper layer) exists in a steady-state equilibrium, forming a protective barrier against the external environment. Once the protective barrier is broken, the normal (physiologic) process of wound healing is immediately set in motion. The classic model of wound healing is divided into four sequential, yet overlapping, phases: (1) hemostasis, (2) inflammatory, (3) proliferative and (4) remodeling. Upon injury to the skin, a set of complex biochemical events takes place in a closely orchestrated cascade to repair the damage. Within minutes post-injury, platelets (thrombocytes) aggregate at the injury site to form a fibrin clot. This clot acts to control active bleeding (hemostasis).

In the inflammatory phase, bacteria and debris are phagocytosed and removed, and factors are released that cause the migration and division of cells involved in the proliferative phase.

The proliferative phase is characterized by angiogenesis, collagen deposition, granulation tissue formation, epithelialization, and wound contraction. In angiogenesis, new blood vessels are formed by vascular endothelial cells.[5] In fibroplasia and granulation tissue formation, fibroblasts grow and form a new, provisional extracellular matrix (ECM) by excreting collagen and fibronectin. Concurrently, re-epithelialization of the epidermis occurs, in which epithelial cells proliferate and ‘crawl’ atop the wound bed, providing cover for the new tissue.

In contraction, the wound is made smaller by the action of myofibroblasts, which establish a grip on the wound edges and contract themselves using a mechanism similar to that in smooth muscle cells. When the cells' roles are close to complete, unneeded cells undergo apoptosis.[

In the maturation and remodeling phase, collagen is remodeled and realigned along tension lines and cells that are no longer needed are removed by apoptosis.

In one embodiment, the methods include administering a Granzyme B inhibitor to the subject for a time and in an amount sufficient to promote wound healing, thereby promoting wound healing in the subject having a wound. In one embodiment, the methods include applying a Granzyme B inhibitor to the wound for a time and in an amount sufficient to promote wound healing, thereby promoting wound healing in the subject having a wound.

In one embodiment, the wound is an acute wound.

In one embodiment, the wound is a “chronic wound” or “recurring wound”. As used herein, the terms “chronic wound” and “recurring wound” refer to wounds that have failed to proceed through an orderly and timely reparative process to produce anatomic and functional integrity of the injured site. Chronic wounds are those that are detained in one or more of the phases of wound healing. For example, in acute wounds, there is a precise balance between production and degradation of molecules such as collagen; in chronic wounds this balance is lost and degradation plays too large a role. In one embodiment, a “chronic wound” or a “recurring wound” is a wound that has not shown significant healing in about four weeks (or about 15, 16, 17, 18, 19, 20, 21, 22, 23, 24, 25, 26, 27, 28, 29, 30, 31, 32, 33, 34, or about 35 days), or which have not completely healed in about eight weeks (or about 40, 41, 42, 43, 44, 45, 46, 47, 48, 49, 50, 51, 52, 53, 54, 55, 56, 57, 58, 59, 60, 61, 62, 63, 64, or about 65 days). Chronic wounds, as used herein, also refer to wounds in which inflammation has not resolved, wounds that have not been restored to greater than 80% of the injured tissue's original tensile strength, wounds in which decorin is reduced and/or collagen remains disorganized and/or wounds in which there is an absence of collagen thick bundle formation.

Chronic wounds can result from traumatic injury, diabetes, peripheral vascular disease, vein abnormalities, complications following surgery, lymphedema and many other conditions that compromise circulation. In one embodiment, the chronic wound is a skin wound, however those skilled in the art will appreciate that wounds may occur in other epithelial tissue. As a non-limiting example, the term “wound” encompasses, without limitation, skin ulcers, which can include: venous skin ulcers, arterial skin ulcers, pressure ulcers, and diabetic skin ulcers. Wounds can also include, without limitation, lacerations, and burns (e.g. heat, chemical, radioactivity, UV burns) of the epithelial tissue. In one embodiment, a chronic skin wound is a pressure ulcer or bed sore.

Use of an “effective amount” of a Granzyme B inhibitor of the present invention (and therapeutic compositions comprising such agents) is an amount effective, at dosages and for periods of time necessary to achieve the desired result.

For example, an effective amount of a Granzyme B inhibitor may vary according to factors such as the disease state, age, sex, reproductive state, and weight, and the ability of the inhibitor to elicit a desired response in the subject. Dosage regimens may be adjusted to provide the optimum response. For example, several divided doses may be provided daily or the dose may be proportionally reduced as indicated by the exigencies of the situation.

An “effective amount” or “therapeutically effective amount” of a Granzyme B inhibitor, e.g., which inhibits extracellular proteoglycan cleavage, e.g., decorin cleavage, is an amount sufficient to produce the desired effect, e.g., an inhibition of extracellular proteoglycan cleavage, e.g., decorin cleavage, in comparison to the normal level of extracellular proteoglycan cleavage, e.g., decorin cleavage, detected in the absence of the Granzyme B inhibitor. Inhibition of extracellular proteoglycan cleavage, e.g., decorin cleavage, is achieved when the value obtained with a Granzyme B inhibitor relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring and determining extracellular proteoglycan cleavage, e.g., decorin cleavage, are known in the art and described herein and include, e.g., examination of protein or RNA levels using techniques known to those of skill in the art such as dot blots, Northern blots, in situ hybridization, ELISA, immunoprecipitation, enzyme function, as well as phenotypic assays described herein and known to those of ordinary skill in the art.

In certain embodiments of the invention, the methods and uses for promoting wound healing in a subject having a chronic wound include administering or applying a Granzyme B inhibitor for a time and in an amount sufficient such that cleavage of an extracellular matrix protein, e.g., an extracellular proteoglycan, is inhibited. The extracellular matrix protein, e.g., an extracellular proteoglycan, may be selected from the group consisting of decorin, biglycan, betaglycan, syndecan, brevican, fibromodulin, fibrillin-1, fibrillin-2, and fibulin-2. In one embodiment, the extracellular matrix protein, e.g., an extracellular proteoglycan, is decorin.

In other embodiments, the methods and uses for promoting wound healing in a subject having a chronic wound include administering or applying a Granzyme B inhibitor for a time and in an amount sufficient such that release of TGFβ or other growth factor or cytokine bound to an extracellular matrix protein, e.g., an extracellular proteoglycan, selected from the group consisting of decorin, biglycan, betaglycan, syndecan, brevican, fibrillin-1, fibrillin-2, and fibulin-2 is inhibited. In one embodiment, release of TGFβ bound to decorin is inhibited.

In another aspect, the present invention provides methods of preventing skin tearing of a subject. Skin tearing may be associated with a wound, such as a chronic wound, such as a chronic skin wound, or aging. The methods include, applying a Granzyme B inhibitor to the skin of the subject for a time and in an amount sufficient to prevent skin tearing, thereby preventing skin tearing in the subject.

In certain embodiments of the invention, the methods and uses for preventing skin tearing in a subject include applying a Granzyme B inhibitor for a time and in an amount sufficient such that cleavage of an extracellular matrix protein, e.g., an extracellular proteoglycan, is inhibited. The extracellular matrix protein, e.g. an extracellular proteoglycan, may be selected from the group consisting of decorin, biglycan, betaglycan, syndecan, brevican, fibromodulin, fibrillin-1, fibrillin-2, and fibulin-2. In one embodiment, the extracellular matrix protein, e.g., an extracellular proteoglycan, is decorin.

In other embodiments, the methods and uses for preventing skin tearing in a subject include applying a Granzyme B inhibitor for a time and in an amount sufficient such that release of TGF bound to an extracellular matrix protein, e.g., an extracellular proteoglycan, selected from the group consisting of decorin, biglycan, betaglycan, syndecan, brevican, fibromodulin, fibrillin-1, fibrillin-2, and fibulin-2 is inhibited. In one embodiment, release of TGF bound to decorin is inhibited.

As used herein, a “skin tear” is a traumatic wound occurring as a result of friction and/or shearing forces which separate the epidermis from the dermis, or separate both the epidermis and the dermis from underlying structures. In one embodiment, the skin tear is a wound of an extremity. In one embodiment, the skin tear is a recurring or chronic skin tear, e.g., a skin tear that had previously occurred in the same area within about 60, 65, 70, 75, 80, 85, 90, 95, 100, 105, or about 110 days prior.

In another aspect, the present invention provides methods of inhibiting hypertrophic scarring of a wound. The methods include, applying a Granzyme B inhibitor to the skin of the subject for a time and in an amount sufficient to prevent skin hypertrophic scarring of a wound, thereby inhibiting hypertrophic scarring of a wound.

In certain embodiments of the invention, the methods and uses for inhibiting hypertrophic scarring of a wound include applying a Granzyme B inhibitor to the wound for a time and in an amount sufficient such that cleavage of an extracellular matrix protein, e.g., an extracellular proteoglycan, is inhibited. The extracellular matrix protein, e.g., an extracellular proteoglycan, may be selected from the group consisting of decorin, biglycan, betaglycan, syndecan, brevican, fibromodulin, fibrillin-1, fibrillin-2, and fibulin-2. In one embodiment, the extracellular matrix protein, e.g. an extracellular proteoglycan, is decorin.

In other embodiments, the methods and uses for inhibiting hypertrophic scarring of a wound in a subject include applying a Granzyme B inhibitor for a time and in an amount sufficient such that release of TGFβ bound to an extracellular matrix protein, e.g., an extracellular proteoglycan, selected from the group consisting of decorin, biglycan, betaglycan, syndecan, brevican, fibromodulin, fibrillin-1, fibrillin-2, and fibulin-2 is inhibited. In one embodiment, release of TGFβ bound to decorin is inhibited.

As used herein, the term “hypertrophic scarring” refers to a cutaneous condition characterized by deposits of excessive amounts of collagen which gives rise to a raised scar, but not to the degree observed with keloids. Like keloids, however, they form most often at the sites of pimples, body piercings, cuts and burns. They often contain nerves and blood vessels. They generally develop after thermal or traumatic injury that involves the deep layers of the dermis. In addition, hypertrophic scars lack decorin and have elevated levels of TGFβ.

In other aspect, the present invention provides methods for increasing collagen organization in the skin of a subject in need thereof. The methods include applying a Granzyme B inhibitor to the skin of the subject in an amount and for a time sufficient to increase collagen organization in the subject, thereby increasing collagen organization in the skin of the subject.

A subject in need of increasing collagen organization in the skin is a subject have frail skin due to, for example, age, disease, e.g., diabetes, immobilization, medication (e.g., long-term corticosteroid use), dehydration, and those having had a previous skin tear within about 60, 65, 70, 75, 80, 85, 90, 95, 100, 105, or about 110 days prior.

In certain embodiment, the methods and uses for increasing collagen organization include applying a Granzyme B inhibitor to the skin of the subject in an amount and for a time sufficient such that cleavage of an extracellular matrix protein, e.g., an extracellular proteoglycan, is inhibited. The extracellular matrix protein, e.g., an extracellular proteoglycan, may be selected from the group consisting of decorin, biglycan, betaglycan, syndecan, brevican, fibromodulin, fibrillin-1, fibrillin-2, and fibulin-2. In one embodiment, the extracellular matrix proteoglycan is decorin

In other embodiments, the methods and uses for increasing collagen organization include applying a Granzyme B inhibitor for a time and in an amount sufficient such that release of TGFβ bound to an extracellular matrix protein, e.g. an extracellular proteoglycan, selected from the group consisting of decorin, biglycan, betaglycan, syndecan, brevican, fibromodulin, fibrillin-1, fibrillin-2, and fibulin-2 is inhibited. In one embodiment, release of TGFβ bound to decorin is inhibited.

In other aspect, the present invention provides methods for increasing the tensile strength of a healing or healed skin wound, e.g., a chronic skin wound, of a subject. The methods include applying a Granzyme B inhibitor to the skin of the subject in an amount and for a time sufficient to increase the tensile strength of the healing or healed skin wound of the subject.

In certain embodiment, the methods and uses for increasing the tensile strength of a healing or healed skin wound of a subject include applying a Granzyme B inhibitor to the skin of the subject in an amount and for a time sufficient such that cleavage of an extracellular matrix protein, e.g., an extracellular proteoglycan is inhibited. The extracellular matrix protein, e.g., an extracellular proteoglycan, may be selected from the group consisting of decorin, biglycan, betaglycan, syndecan, brevican, fibrillin-1, fibrillin-2, and fibulin-2. In one embodiment, the extracellular matrix protein, e.g. an extracellular proteoglycan, is decorin.

In other embodiments, the methods and uses for increasing the tensile strength of a healing or healed skin wound, e.g. a chronic skin wound include applying a Granzyme B inhibitor for a time and in an amount sufficient such that release of TGFβ bound to an extracellular matrix protein, e.g., an extracellular proteoglycan, selected from the group consisting of decorin, biglycan, betaglycan, syndecan, brevican, fibromodulin, fibrillin-1, fibrillin-2, and fibulin-2 is inhibited. In one embodiment, release of TGFβ bound to decorin is inhibited.

A “healing wound” is a wound in which clotting has occurred, a wound in which temporary replacement of cells and extracellular matrix has occurred, a wound in which resolution of inflammation has occurred, and/or a wound in which synthesis and organization of cells and extracellular matrix in a manner that restores tissue functionality and structure has occurred.

In another aspect, the present invention provides methods for inhibiting release of a cytokine, e.g., transforming growth factor-β (TGF-β), bound to an extracellular matrix protein, e.g., an extracellular proteoglycan, e.g., release of active TGF-β. The methods include, contacting the extracellular matrix protein, e.g. an extracellular proteoglycan, with a Granzyme B inhibitor, thereby inhibiting release of the cytokine, e.g., TGFβ, bound to an extracellular matrix protein, e.g. an extracellular proteoglycan. The methods may also involve inhibiting a cleavage site in the extracellular matrix protein, e.g., an extracellular proteoglycan. Optionally, the cleavage occurs in any one of the following peptide sequences: Asp⁹¹Thr-Thr-Leu-Leu-Asp (SEQ ID NO: 1); or Asp⁵⁵⁸Ala-Ser-Leu-Phe-Thr (SEQ ID NO:2); or Asp³¹Glu-Ala-Ser-Gly (SEQ ID NO:3); or Asp⁶⁹Leu-Gly-Asp-Lys (SEQ ID NO:4); or Asp⁸²Thr-Thr-Leu-Leu-Asp (SEQ ID NO:5); or Asp²⁶¹Asn-Gly-Ser-Leu-Ala (SEQ ID NO:6).

The methods and uses of inhibiting release of a cytokine, e.g. TGFβ, bound to an extracellular matrix protein, e.g., an extracellular proteoglycan, may be performed in vitro or in vivo. The extracellular matrix protein, e.g. an extracellular proteoglycan, may be selected from the group consisting of decorin, biglycan, betaglycan, syndecan, brevican, fibromodulin, fibrillin-1, fibrillin-2, and fibulin-2. In one embodiment, the extracellular matrix protein, e.g. an extracellular proteoglycan, is decorin.

In another aspect, the present invention provides methods for inhibiting extracellular matrix protein degradation. The methods include contacting the extracellular matrix protein, e.g., an extracellular proteoglycan, with a Granzyme B inhibitor, wherein the release of a sequestered cytokine, e.g. TGFβ, is inhibited, thereby inhibiting extracellular matrix protein degradation.

The methods and uses of inhibiting degradation of an extracellular matrix protein, e.g., an extracellular proteoglycan, may be performed in vitro or in vivo. The extracellular matrix protein, e.g. an extracellular proteoglycan, may be selected from the group consisting of decorin, biglycan, betaglycan, syndecan, brevican, fibromodulin, fibrillin-1, fibrillin-2, and fibulin-2. In one embodiment, the extracellular matrix protein, e.g., an extracellular proteoglycan, is decorin.

In yet another aspect, the present invention provides methods of inhibiting extracellular decorin cleavage. The methods include, contacting the extracellular decorin with a Granzyme B inhibitor, thereby inhibiting extracellular decorin cleavage.

The methods and uses of inhibiting decorin cleavage may be performed in vitro or in vivo. In certain embodiments, the methods include contacting a cell, such as a skin cell, with a Granzyme B inhibitor such that the expression and/or activity of decorin are increased in the epidermal-dermal junction of the skin.

The Granzyme B inhibitor for use in the methods, uses and compositions described herein may be a nucleic acid, a peptide, an antibody, such as a humanized antibody, or a small molecule. Granzyme B inhibitors for use in any of the methods, uses, and compositions of the invention are described in detail below.

The term “subject” or “patient” is intended to include mammalian organisms. Examples of subjects or patients include humans and non-human mammals, e.g., non-human primates, dogs, cows, horses, pigs, sheep, goats, cats, mice, rabbits, rats, and transgenic non-human animals. In specific embodiments of the invention, the subject is a human.

The term “administering” includes any method of delivery of a Granzyme B inhibitor or a pharmaceutical composition comprising a Granzyme B inhibitor into a subject's system or to a particular region in or on a subject. In certain embodiments, a moiety is administered topically, intravenously, intramuscularly, subcutaneously, intradermally, intranasally, orally, transcutaneously, intrathecal, intravitreally, intracerebral, or mucosally.

In one embodiment, the administration of the Granzyme B inhibitor is a local administration, e.g., administration to the site of a wound, e.g., a chronic skin wound. In one embodiment the administration of the Granzyme B inhibitor is topical administration to the site of a wound, e.g., a chronic skin wound.

As used herein, the term “applying” refers to administration of a Granzyme B inhibitor that includes spreading, covering (at least in part), or laying on of the inhibitor. For example, a Granzyme B inhibitor may be applied to the skin of a subject or applied to a wound by spreading or covering the skin with an inhibitor. In addition, a Granzyme B inhibitor may be applied to the skin or wound using, for example, a wound covering comprising the inhibitor.

As used herein, the term “contacting” (i.e., contacting a protein, a cell, e.g., a host cell, or a subject with a Granzyme B inhibitor) includes incubating the Granzyme B inhibitor and the, e.g., cell, together in vitro (e.g., adding the moiety to cells in culture) as well as administering the moiety to a subject such that the moiety and cells or tissues of the subject are contacted in vivo.

As used herein, the terms “treating” or “treatment” refer to a beneficial or desired result including, but not limited to, alleviation or amelioration of one or more symptoms, diminishing the extent of a disorder, stabilized (i.e., not worsening) state of a disorder, amelioration or palliation of the disorder, whether detectable or undetectable. “Treatment” can also mean prolonging survival as compared to expected survival in the absence of treatment.

### II. Granzyme B Inhibitors

A Granzyme B inhibitor for use in any of the compositions, methods and uses of the present invention may be a nucleic acid molecule, a peptide, an antibody, such as a humanized antibody or a camelid antibody, or a small molecule.

Many Granzyme B inhibitors are known to a person of skill in the art and are, for example, described in international patent application published under WO 03/065987 and United States patent application published under US 2003/0148511; Willoughby et al., 2002; Hill et al., 1995; Sun J. et al., 1996; Sun J. et al., 1997; Bird et al., 1998; Kam et al., 2000; and Mahrus and Craik, 2005.

A Granzyme B inhibitor for use in any of the compositions, methods and uses of the present invention may be a nucleic acid molecule, a peptide, an antibody, such as a humanized antibody or a camelid antibody, or a small molecule.

In one embodiment, a Granzyme B inhibitor is selected from the group consisting of
2S,5S)—N-((2H-tetrazol-5-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((R)-3-methyl-2-(pyridin-2-yl)butanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide;
(2S,5S)—N-((1H-1,2,4-triazol-3-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-pyrazol-3-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-pyrazol-4-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-imidazol-4-yl)methyl)-5-((2S,3S)-2-acetamido-3-methylpentanamide)-4-oxo-1,2,4,5,6,7-hexahydroazepino[3,2,1-hi]indole-2-carboxamide;
2S,5S)-5-((2S,3S)-2-acetamido-3-methylpentanamido)-4-oxo-N-(thiazol-5-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(isoxazol-3-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-4-oxo-N-(thiazol-2-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(isoxazol-5-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-4-oxo-N-(thiazol-4-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyrimidin-5-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridazin-4-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-2-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-3-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-4-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(imidazo[1,2-a]pyrimidin-2-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-N-((3a,7a-dihydrobenzo[d]thiazol-2-yl)methyl)-4-oxo-1,2,4,5,6,7-hexahydroazepino[3,2,1-hi]indole-2-carboxamide;
(2S,5S)—N-((2H-tetrazol-5-yl)methyl)-5-((R)-3-methyl-2-(pyridin-2-yl)butanamido)-4-oxo-1,2,4,5,6,7-hexahydroazepino[3,2,1-hi]indole-2-carboxamide;
(2S,5S)—N-((2H-tetrazol-5-yl)methyl)-5-((S)-3-methyl-2-(pyridin-2-yl)butanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(2,3-difluorophenyl)acetamido)-3-methylpentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide;
(R)—N-((2S,5S)-2-((1H-1,2,3-triazol-4-yl)methylcarbamoyl)-4-oxo-1,2,4,5,6,7-hexahydroazepino[3,2,1-hi]indol-5-yl)-3-acetyl-5,5-dimethylthiazolidine-4-carboxamide;
(2S,5S)—N-((1H-1,2,3-triazol-4-yl)methyl)-5-((2S,3S)-3-methyl-2-(2-oxopyrrolidin-1-yl)pentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-(2-cyclopentylacetamido)-4-oxo-1,2,4,5,6,7-hexahydroazepino[3,2,1-hi]indole-2-carboxamide;
(2S,5S)—N-((1H-1,2,3-triazol-4-yl)methyl)-5-((S)-2-acetamido-2-cyclopropylacetamido)-4-oxo-1,2,4,5,6,7-hexahydroazepino[3,2,1-hi]indole-2-carboxamide;
(2S,5S)—N-((1H-1,2,3-triazol-4-yl)methyl)-5-((S)-2-acetamido-2-cyclopentylacetamido)-4-oxo-1,2,4,5,6,7-hexahydroazepino[3,2,1-hi]indole-2-carboxamide;
Bio-x-IEPD^{P}-(OPh)₂;
azepino[3,2,1-hi]indole-2-carboxamide;
(4S)-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
(4S)—[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid;
5-chloro-4-oxo-3-[2-[2-(phenylmethoxycarbonylamino) propanoylamino]propanoylamino]pentanoic acid;
5-chloro-4-oxo-2-[2-[2-(phenylmethoxycarbonylamino) propanoylamino]propanoylamino]pentanoic acid;
ZINC05723764;
ZINC05723787;
ZINC05316154;
ZINC05723499;
ZINC05723646;
ZINC05398428;
ZINC05723503;
ZINC05723446;
ZINC05317216;
ZINC05315460;
ZINC05316859;
ZINC05605947;
an isocoumarin;
a peptide chloromethyl ketone;
a peptide phosphonate;
a Granzyme B inhibitory nucleic acid molecule;
an anti-Granzyme B antibody;
an inhibitory Granzyme B peptide;
a SerpB9 polypeptide, or fragment thereof;
5-chloro-4-oxo-2-[2-[2-(phenylmethoxycarbonylamino) propanoylamino]propanoylamino]pentanoic acid;
Ac-IEPD-CHO;
(4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
Ac-IETD-CHO;
(4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid;
(2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino) propanoylamino]propanoylamino]pentanoic acid;
5-chloro-4-oxo-2-[2-[2-(phenylmethoxycarbonylamino) propanoylamino]propanoylamino]pentanoic acid;
(4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
(4S)-[[(2S,3S)-2-acetamido-3-methylpentanoyl]amino]-5-[[(2S,3S)-3-hydroxy-1-[[(2S)-hydroxy-1,4-dioxobutan-2-yl]amino]-1-oxobutan-2-yl]amino]-5-oxopentanoic acid,
a Serp2 polypeptide, or fragment thereof;
a CrmA polypeptide or fragment thereof; and
a SerpinA3 polypeptide or fragment thereof.

In another embodiment, a Granzyme B inhibitor suitable for use in the methods, compositions, and uses of the invention includes, for example, Z-AAD-CMK (IUPAC name: 5-chloro-4-oxo-2-[2-[2-(phenylmethoxycarbonylamino) propanoylamino]propanoylamino]pentanoic acid) MF: C19H24ClN3O7 CID: 16760474; Ac-IEPD-CHO; Granzyme B Inhibitor IV or Caspase-8 inhibitor III (IUPAC: (4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid) MF: C22H34N4O9 CID: 16760476; and Ac-IETD-CHO; Caspase-8 Inhibitor I or Granzyme B Inhibitor II (IUPAC: (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid) MF: C21H34N4O10 CID: and 16760475.

In yet another embodiment, a Granzyme B inhibitor for use in the methods, compositions, and uses of the invention may include any one or more of the following: Granzyme B inhibitor is selected from one or more of the following: Azepino[3,2,1-hi]indole-2-carboxamide, 5-[[(2S,3S)-2-[(2-benzo[b]thien-3-ylacetyl)amino]-3-methyl-1-oxopentyl]amino]-1,2,4,5,6,7-hexahydro-4-oxo-N-(1H-1,2,3-triazol-5-ylmethyl)-, (2S,5S)-(compound 20 from Willoughby et al. (2002) Bioorganic & Medicinal Chemistry Letters 12:2197-2200) referred to herein as Willoughby 20; Bio-x-IEPD^{P}-(OPh)₂; (2S,5S)-5-[(N-acetyl-L-isoleucyl)amino]-4-oxo-N-(1H-tetraazol-5-ylmethyl)-1,2,4,5,6,7-hexahydroazepino[3,2,1-hi]indole-2-carboxamide; (2S,5S)-5-[(N-acetyl-L-isoleucyl)amino]-4-oxo-N-(1H-1,2,3-triazol-4-ylmethyl)-1,2,4,5,6,7-hexahydroazepino[3,2,1-hi]indole-2-carboxamide; (2S,5S)-5-([(2R)-3-methyl-2-pyridin-2-ylbutanoyl]amino)-4-oxo-N-(1H-1,2,3-triazol-4-ylmethyl)-1,2,4,5,6,7-hexahydroazepino[3,2,1-hi]indole-2-carboxamide; (2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino) propanoylamino]propanoylamino]pentanoic acid; 5-chloro-4-oxo-2-[2-[2-(phenylmethoxycarbonylamino) propanoylamino]propanoylamino]pentanoic acid; (4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid; (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid, a protease inhibitor-9 or derivatives thereof, CrmA, serp-2, ZIINC05723764, ZINC05723787, ZINC05316154, ZINC05723499, ZINC05723646, ZINC05398428, ZINC05723503, ZINC05723446, ZINC05317216, ZINC05315460, ZINC05316859, and ZINC05605947.

Alternatively, the Granzyme B inhibitor may be selected from one or more of the following: Willoughby 20, NCI 644752, NCI 644777, ZINC05317216, and NCI 630295. Granzyme B inhibitors may include, but are not limited to, nucleic acids (for example, antisense oligonucleotides, siRNA, RNAi, etc.), peptides and small molecules.

Optionally, the Granzyme B inhibitor used herein may be selected from one of the examples detailed herein, which includes but is not limited to one or more of the following: Azepino[3,2,1-hi]indole-2-carboxamide, 5-[[(2S,3S)-2-[(2-benzo[b]thien-3-ylacetyl)amino]-3-methyl-1-oxopentyl]amino]-1,2,4,5,6,7-hexahydro-4-oxo-N-(1H-1,2,3-triazol-5-ylmethyl)-, (2S,5S)-(compound 20 from Willoughby et al. (2002) Bioorganic & Medicinal Chemistry Letters 12:2197-2200) referred to herein as Willoughby 20; Bio-x-IEPD^{P}-(OPh)₂; (2S,5S)-5-[(N-acetyl-L-isoleucyl)amino]-4-oxo-N-(1H-tetraazol-5-ylmethyl)-1,2,4,5,6,7-hexahydroazepino[3,2,1-hi]indole-2-carboxamide; (2S,5S)-5-[(N-acetyl-L-isoleucyl)amino]-4-oxo-N-(1H-1,2,3-triazol-4-ylmethyl)-1,2,4,5,6,7-hexahydroazepino[3,2,1-hi]indole-2-carboxamide; (2S,5S)-5-{[(2R)-3-methyl-2-pyridin-2-ylbutanoyl]amino}-4-oxo-N-(1H-1,2,3-triazol-4-ylmethyl)-1,2,4,5,6,7-hexahydroazepino[3,2,1-hi]indole-2-carboxamide; (2S,5S)-4-oxo-5-{[N-(phenylacetyl)-L-isoleucyl]amino}-N-(1H-1,2,3-triazol-4-ylmethyl)-1,2,4,5,6,7-hexahydroazepino[3,2,1-hi]indole-2-carboxamide; 5-chloro-4-oxo-3-[2-[2-(phenylmethoxycarbonylamino) propanoylamino]propanoylamino]pentanoic acid; 5-chloro-4-oxo-2-[2-[2-(phenylmethoxycarbonylamino) propanoylamino]propanoylamino]pentanoic acid; (4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid; (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid, protease inhibitor-9 or derivatives thereof, CrmA, serp-2, ZINC05723764, ZINC05723787, ZINC05316154, ZINC05723499, ZINC05723646, ZINC05398428, ZINC05723503, ZINC05723446, ZINC05317216, ZINC05315460, ZINC05316859, and ZINC05605947. Alternatively, the Granzyme B inhibitor may be selected from one or more of the following: Willoughby 20, NCI 644752, NCI 644777, ZINC05317216, and NCI 630295.

In one embodiment, a Granzyme B inhibitor for use in any of the compositions, uses and methods of the invention is a nucleic acid molecule.

As used herein, the term “nucleic acid” refers to a deoxyribonucleotide or ribonucleotide polymer in either single- or double-stranded form, and any chemical modifications thereof. Such modifications include, but are not limited to backbone modifications, methylations, and unusual base-pairing combinations. As detailed herein, the term “nucleic acid” includes, without limitation, RNAi technologies. For example, RNA compounds used to inhibit Granzyme B may be small interfering RNA (siRNA) compounds.

In one embodiment, a Granzyme B inhibitor for use in the compositions, uses and methods of the invention is an interfering nucleic acid molecule.

The term “interfering nucleic acid molecule” or “interfering nucleic acid” as used herein includes single-stranded RNA (e.g., mature miRNA. ssRNAi oligonucleotides, ssDNAi oligonucleotides), double-stranded RNA (i.e., duplex RNA such as siRNA, Dicer-substrate dsRNA, shRNA, aiRNA, or pre-miRNA), self-delivering RNA (sdRNA; see, e.g. U.S. Patent Publication Nos. 200913120341, 200913120315, and 201113069780, the entire contents of all of which are incorporated herein by reference), a DNA-RNA hybrid (see, e.g., PCT Publication No. WO 2004/078941), or a DNA-DNA hybrid (see, e.g., PCT Publication No. WO 2004/104199) that is capable of reducing or inhibiting the expression (and, thus, the activity) of a target gene or sequence (e.g., by mediating the degradation or inhibiting the translation of mRNAs which are complementary to the interfering RNA sequence) when the interfering nucleic acid is in the same cell as the target gene or sequence. Interfering nucleic acid thus refers to a single-stranded nucleic acid molecules that are complementary to a target mRNA sequence or to the double-stranded RNA formed by two complementary strands or by a single, self-complementary strand. Interfering nucleic acids may have substantial or complete identity to the target gene or sequence, or may comprise a region of mismatch (i.e., a mismatch motif). The sequence of the interfering nucleic acids can correspond to the full-length target gene, or a subsequence thereof (e.g. the gene for Granzyme B, the nucleotide and amino acid sequence of which is known and may be found in for example GenBank Accession No, GI:221625527, the entire contents of which are incorporated herein by reference, and SEQ ID NO:8). Preferably, the interfering nucleic acid molecules are chemically synthesized. The disclosures of each of the above patent documents are herein incorporated by reference in their entirety for all purposes.

As used herein, the term “mismatch motif” or “mismatch region” refers to a portion of an interfering nucleic acid (e.g., siRNA) sequence that does not have 100% complementarity to its target sequence. An interfering nucleic acid may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

An interfering nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an interfering nucleic acid is an antisense nucleic acid and can hydrogen bond to the sense nucleic acid.

In one embodiment, an interfering nucleic acid of the invention is a “small-interfering RNA” or “an siRNA” molecule. In another embodiment, an interfering nucleic acid molecules of the invention is a “self-delivering RNA” or “sdRNA” molecule. In one embodiment, an interfering nucleic acid of the invention mediates RNAi. RNA interference (RNAi) is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell T R, and Doering T L. 2003. Trends Microbiol. 11:37-43; Bushman F. 2003. Mol. Therapy. 7:9-10; McManus M T and Sharp P A. 2002. Nat Rev Genet. 3:737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs or Ambion. In one embodiment one or more of the chemistries described herein for use in antisense RNA can be employed in molecules that mediate RNAi.

Interfering nucleic acid includes, e.g., siRNA and sdRNA, of about 10-60, 10-50, or 10-40 (duplex) nucleotides in length, more typically about 8-15, 10-30, 10-25, or 10-25 (duplex) nucleotides in length, about 10-24, (duplex) nucleotides in length (e.g., each complementary sequence of the double-stranded siRNA is 10-60, 10-50, 10-40, 10-30, 10-25, or 10-25 nucleotides in length, about 10-24, 11-22, or 11-23 nucleotides in length, and the double-stranded siRNA is about 10-60, 10-50, 10-40, 10-30, 10-25, or 10-25 base pairs in length). siRNA and sdRNA duplexes may comprise 3′-overhangs of about 1, 2, 3, 4, 5, or about 6 nucleotides and 5′-phosphate termini. Examples of siRNA and sdRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA (or sdRNA) molecule. As used herein, the terms “siRNA” and “sdRNA’ include RNA-RNA duplexes as well as DNA-RNA hybrids (see, e.g., PCT Publication No. WO 2004/078941).

Preferably, siRNA and sdRNA are chemically synthesized. siRNA and sdRNA can also be generated by cleavage of longer dsRNA (e.g., dsRNA about 5, about 10, about 15, about 20, about 25, or greater nucleotides in length) with the E. coli RNase III or Dicer. These enzymes process the dsRNA into biologically active siRNA (see, e.g., Yang et al., Proc. Natl. Acad. Sci: USA, 99:9942-9947 (2002); Calegari et al., Proc. Natl. Acad. Sci. USA, 99:14236 (2002); Byrom et al. Ambion TechNotes, 10(1):4-6 (2003); Kawasaki et al., Nucleic Acids Res., 31:981-987 (2003); Knight et al., Science, 293:2269-2271 (2001); and Robertson et al. J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA or sdRNA may be encoded by a plasmid (e.g., transcribed as sequences that automatically fold into duplexes with hairpin loops).

Given the coding strand sequences encoding Granzyme B known in the art and disclosed herein (SEQ ID NO:8), an interfering nucleic acid of the invention can be designed according to the rules of Watson and Crick base pairing. The interfering nucleic acid molecule can be complementary to the entire coding region of Granzyme B mRNA, but more preferably is an oligonucleotide which is antisense to only a portion of the coding or noncoding region of Granzyme B mRNA. For example, an interfering oligonucleotide can be complementary to the region surrounding the processing site of ubiquitin and Granzyme B mRNA. An interfering RNA oligonucleotide can be, for example, about 5, 10, 15, 20, 25, 30, 35, 40, 45 or 50 nucleotides in length. An interfering nucleic acid of the invention can be constructed using chemical synthesis and enzymatic ligation reactions using procedures known in the art. For example, an interfering nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the interfering nucleic acids include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. To inhibit expression in cells, one or more interfering nucleic acid molecules can be used. Alternatively, an interfering nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The interfering nucleic acids may include any RNA compounds which have sequence homology to the Granzyme B gene and which are capable of modulating the expression of Granzyme B protein. Examples interfering nucleic acids which are capable of modulating expression of Granzyme B are found in: U.S. Pat. No. 6,159,694; U.S. Pat. No. 6,727,064; U.S. Pat. No. 7,098,192; and U.S. Pat. No. 7,307,069, the entire contents of all of which are incorporated herein by reference.

Antisense oligonucleotides directed against Granzyme B have been designed and manufactured by Biognostik (Euromedex, Mundolshei, France) and are described in Hernandez-Pigeon, et al., J. Biol Chem. vol. 281, 13525-13532 (2006) and Bruno, et al., Blood, vol. 96, 1914-1920 (2000).

In another embodiment, a Granzyme B inhibitor for use in the compositions, methods and uses of the invention is a peptide.

As used herein, “peptide” refers to short polymers of amino acids linked by peptide bonds. Those persons skilled in the art will understand that a peptide bond, which is also know in the art as an amide bond, is a covalent chemical bond formed between two molecules when the carboxyl group of one molecule reacts with the amine group of the other molecule, thereby releasing a molecule of water (H₂O). Peptides may be modified in a variety of conventional ways well known to the skilled artisan. Examples of modifications include the following. The terminal amino group and/or carboxyl group of the peptide and/or amino acid side chains may be modified by alkylation, amidation, or acylation to provide esters, amides or substituted amino groups. Heteroatoms may be included in aliphatic modifying groups. This is done using conventional chemical synthetic methods. Other modifications include deamination of glutamyl and asparaginyl residues to the corresponding glutamyl and aspartyl residues, respectively; hydroxylation of proline and lysine; phosphorylation of hydroxyl groups of serine or threonine; and methylation of amino groups of lysine, arginine, and histidine side chains (see, for e.g.: T. E. Creighton. Proteins: Structure and Molecular Properties, W.H. Freeman & Co. San Francisco, Calif., 1983).

In another aspect, one or both, usually one terminus of the peptide, may be substituted with a lipophilic group, usually aliphatic or aralkyl group, which may include heteroatoms. Chains may be saturated or unsaturated. Conveniently, commercially available aliphatic fatty acids, alcohols and amines may be used, such as caprylic acid, capric acid, lauric acid, myristic acid and myristyl alcohol, palmitic acid, palmitoleic acid, stearic acid and stearyl amine, oleic acid, linoleic acid, docosahexaenoic acid, etc. (see, for e.g.: U.S. Pat. No. 6,225,444). Preferred are unbranched, naturally occurring fatty acids between 14-22 carbon atoms in length. Other lipophilic molecules include glyceryl lipids and sterols, such as cholesterol. The lipophilic groups may be reacted with the appropriate functional group on the oligopeptide in accordance with conventional methods, frequently during the synthesis on a support, depending on the site of attachment of the oligopeptide to the support. Lipid attachment is useful where oligopeptides may be introduced into the lumen of the liposome, along with other therapeutic agents for administering the peptides and agents into a host.

Depending upon their intended use, particularly for administration to mammalian hosts, the subject peptides may also be modified by attachment to other compounds for the purposes of incorporation into carrier molecules, changing peptide bioavailability, extending or shortening half-life, controlling distribution to various tissues or the blood stream, diminishing or enhancing binding to blood components, and the like. The prior examples serve as examples and are non-limiting.

Peptides may be prepared in a number of ways. Chemical synthesis of peptides is well known in the art. Solid phase synthesis is commonly used and various commercial synthetic apparatuses are available, for example automated synthesizers by Applied Biosystems Inc., Foster City, Calif.; Beckman; etc. Solution phase synthetic methods may also be used, particularly for large-scale productions.

Peptides may also be present in the form of a salt, generally in a salt form which is pharmaceutically acceptable. These include inorganic salts of sodium, potassium, lithium, ammonium, calcium, magnesium, iron, zinc, copper, manganese, and the like. Various organic salts of the peptide may also be made with, including, but not limited to, acetic acid, propionic acid, pyruvic acid, maleic acid, succinic acid, tartaric acid, citric acid, benozic acid, cinnamic acid, salicylic acid, etc.

Peptides can also be made intracellularly in cells by introducing into the cells an expression vector encoding the peptide. Such expression vectors can be made by standard techniques. The peptide can be expressed in intracellularly as a fusion with another protein or peptide (e.g., a GST fusion). Synthesized peptides can then be introduced into cells by a variety of means known in the art for introducing peptides into cells (e.g., liposome and the like).

In one embodiment, a peptide for use in the methods, compositions, and uses of the invention is a serpin. Serpins are a group of naturally occurring proteins that inhibit serine proteases. In one embodiment, the serpin binds to Granzyme B and has Granzyme B inhibitory function.

In one embodiment the Granzyme B inhibitor is a P19 peptide, or a Granzyme B inhibitory fragment thereof (see, e.g., U.S. Patent Publication No. 2003/0148511, the entire contents of which are incorporated herein by reference). P19, also known as SerpinB9 is a human serpin that inhibits Granzyme B (see, e.g., review in Bird, 1999 Immunol. Cell Biol. 77, 47-57). The amino acid and nucleotide sequence of SerpinB9 are known and may be found in, for example, Genbank Accession No. GI:223941859, the entire contents of which are incorporated herein by reference, and SEQ ID NOs:9 and 10. In one embodiment, the peptide is SerpinB9 and comprises pan or all of the sequence from SerpinB9 that binds directly to Granzyme B, i.e. GTEAAASSCFVAECCMESG (SEQ. ID NO: 11). This sequence contains the “reactive center” or “reactive center loop” of SerpinB9. In another embodiment, the Granzyme B inhibitor, e.g., a SerpinB9 peptide comprises the amino acid sequence selected from the group consisting of VEVNEEGTEAAAASSCFVVAECCMESGPRFCADHPFL (SEQ ID NO: 18); VEVNEEGTEAAAASSCFVVADCCMESGPRFCADHPFL (SEQ ID NO:19); VEVNEEGTEAAAASSCFVVAACCMESGPRFCADHPFL (SEQ ID NO:20); and VEVNEEGREAAAASSCFVVAECCMESGPRFCADHPFL (SEQ ID NO:21)

In another embodiment, the Granzyme B inhibitor is a Serpina3n peptide, or a Granzyme B inhibitory fragment thereof. Serpina3n is also known as SerpinA3. The amino acid and nucleotide sequence of SerpinA3 are known and may be found in, for example, Genbank Accession No. GI:73858562, the entire contents of which are incorporated herein by reference, and SEQ ID NOs: 12 and 13.

In another embodiment, the Granzyme B inhibitor is the cowpox virusprotein, CrmA peptide, or a Granzyme B inhibitory fragment thereof (see, e.g. Quan, et al. (1995) 270, 10377-10379) (the amino acid and nucleotide sequences of CrmA are set forth in SEQ ID NOs: 14 and 15). In one embodiment, a Granzyme B inhibitor is a CrmA peptide comprising the amino acid sequence IDVNEEYTEAAAATCALVADCASTVTNEFCADHPFI (SEQ ID NO:22).

In another embodiment, the Granzyme B inhibitor is a Serp2 peptide, or a Granzyme B inhibitory fragment thereof. Serp2 is also known as SerpinA3. The amino acid and nucleotide sequence of SerpinA3 are known and may be found in, for example, Genbank Accession No. GI:58219011, the entire contents of which are incorporated herein by reference, and SEQ ID NOs: 16 and 17.

Other suitable Granzyme B inhibitory peptides for use in any of the methods, compositions, or uses of the invention, include, for example, Z-AAD-CH₂Cl (Z-ALA-ALA-ASP-chloromethylketone), Ac-IEPD-CHO (Ac-Ile-Glu-Pro-Asp-CHO), Ac-IETD-CHO, Ac-AAVALLPAVLLALLAPIETD-cho, and z-IETD-fmk.

In yet another embodiment, a Granzyme B inhibitor for use in the compositions, methods and uses of the invention is an antibody, e.g. an anti-Granzyme B antibody. In one embodiment, the an anti-Granzyme B antibody is a human antibody. In another embodiment, the an anti-Granzyme B antibody is a humanized antibody. In another embodiment, the an anti-Granzyme B antibody is a camelid antibody.

As used herein, the term “antibody” refers to a composition comprising a protein that binds specifically to a corresponding antigen and has a common, general structure of immunoglobulins. The term antibody specifically covers polyclonal antibodies, monoclonal antibodies, dimers, multimers, multispecific antibodies (e.g. bispecific antibodies), and antibody fragments, so long as they exhibit the desired biological activity. The term “antibody” includes, without limitation, camelid antibodies. Antibodies may be murine, human, humanized, chimeric, or derived from other species. Typically, an antibody will comprise at least two heavy chains and two light chains interconnected by disulfide bonds, which when combined form a binding domain that interacts with an antigen. Each heavy chain is comprised of a heavy chain variable region (V_{H}) and a heavy chain constant region (C_{H}). The heavy chain constant region is comprised of three domains, C_{H}1, C_{H}2 and C_{H}3, and may be of the mu (μ), delta (δ), gamma (γ), alpha (α) or epsilon (ε) isotype. Similarly, the light chain is comprised of a light chain variable region (V_{L}) and a light chain constant region (C_{L}). The light chain constant region is comprised of one domain, CL, which may be of the kappa or lambda isotype. The V_{H} and VL regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g. effector cells) and the first component (Clq) of the classical complement system. The heavy chain constant region mediates binding of the immunoglobulin to host tissue or host factors, particularly through cellular receptors such as the Fc receptors (e.g., FcγRI, FcγRII, FcγRIII, etc.). As used herein, antibody also includes an antigen binding portion of an immunoglobulin that retains the ability to bind antigen. These include, as examples, F(ab), a monovalent fragment of V_{L} C_{L} and V_{H} C_{H} antibody domains; and F(ab′)₂ fragment, a bivalent fragment comprising two Fab fragments linked by a disulfide bridge at the hinge region. The term antibody also refers to recombinant single chain Fv fragments (scFv) and bispecific molecules such as, e.g., diabodies, triabodies, and tetrabodies (see, e.g. U.S. Pat. No. 5,844,094).

Antibodies may be produced and used in many forms, including antibody complexes. As used herein, the term “antibody complex” refers to a complex of one or more antibodies with another antibody or with an antibody fragment or fragments, or a complex of two or more antibody fragments.

As used herein, the term “antigen” is to be construed broadly and refers to any molecule, composition, or particle that can bind specifically to an antibody. An antigen has one or more epitopes that interact with the antibody, although it does not necessarily induce production of that antibody.

As used herein the term “epitope” refers to a determinant capable of specific binding to an antibody. Epitopes are chemical features generally present on surfaces of molecules and accessible to interaction with an antibody. Typical chemical features are amino acids and sugar moieties, having three-dimensional structural characteristics as well as chemical properties including charge, hydrophilicity, and lipophilicity. Conformational epitopes are distinguished from non-conformational epitopes by loss of reactivity with an antibody following a change in the spatial elements of the molecule without any change in the underlying chemical structure. The term “epitope” is also understood by those persons skilled in the an as an “antigenic determinant”. For example, an antibody that is secreted by a B cell recognizes only a portion of a macromolecule; the recognized portion is an epitope. The foregoing example is provided solely as an example and is not intended not limit the scope of the term “epitope”. Epitopes are recognized by numerous cell types including B cells and T cells.

As used herein, the term “humanized antibody” refers to an immunoglobulin molecule containing a minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. A humanized antibody will also encompass immunoglobulins comprising at least a portion of an immunoglobulin constant region (Fc), generally that of a human immunoglobulin (Jones et al., 1986; and Reichmann et al. 1988). As used herein the term “antibody fragment” refers to a fragment of an antibody molecule. Antibody fragments can include without limitation: single domains, Fab fragments, and single-chain Fv fragments. As used herein, the term “monoclonal antibody” refers to monospecific antibodies that are the same because they are made by clones of a unique parent cell. As detailed above, the term “antibody” includes without limitation a “monoclonal antibody”.

In one embodiment, a Granzyme B inhibitor is a small molecule.

As used herein, the term “small molecule” refers to a low molecular weight organic compound that binds to a biopolymer such as a protein, a nucleic acid, or a polysaccharide. The foregoing examples of binding partners of a small molecule are non-limiting.

Optionally, the Granzyme B inhibitor used herein may be selected from one of the examples detailed herein, which includes but is not limited to azepine compounds of the following formula:

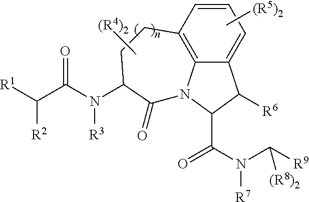

or a pharmaceutically acceptable salt or hydrate thereof, wherein: n is 0, 1, or 2; R¹ and R² are each independently selected from the group consisting of: hydrogen, C₁₋₆alkyl, C₁₋₆alkoxy, C₃₋₆cycloalkyl, aryl, HET and —N(R¹⁰)₂, wherein: (a) said C₁₋₆alkyl, C₁₋₆alkoxy and C₃₋₆cycloalkyl are optionally substituted with 1-3 substituents independently selected from the group consisting of halo and hydroxy; and (b) said aryl and HET are optionally substituted with 1-3 substituents independently selected from the group consisting of: halo, hydroxy and C₁₋₄alkyl, optionally substituted with 1-3 halo groups; or R¹ and R² may be joined together with the carbon atom to which they are attached to form a five or six membered monocyclic ring, optionally containing 1-3 heteroatoms selected from the group consisting of: S, O and N(R¹⁰), wherein said ring is optionally substituted with 1-3 R¹⁰ groups, with the proviso that R¹ and R² are both not hydrogen; each of R³ and R⁷ is independently selected from the group consisting of: hydrogen and C₁₋₄alkyl, optionally substituted with 1-3 halo groups; each of R⁴, R⁵, R⁶ and R⁸ is independently selected from the group consisting of: hydrogen, halo, hydroxy and C₁₋₄alkyl, optionally substituted with 1-3 halo groups; R9 is HET, optionally substituted with 1-3 substituents independently selected from the group consisting of: halo, hydroxy and C₁₋₄alkyl, optionally substituted with 1-3 halo groups; R¹⁰ is selected from the group consisting of: hydrogen, C₁₋₄alkyl and —C(O)C₁₋₄alkyl, said —C(O)C₁₋₄alkyl optionally substituted with N(R¹¹)₂, HET and aryl, said aryl optionally substituted with 1-3 halo groups; R¹¹ is selected from hydrogen and C₁₋₄alkyl, optionally substituted with 1-3 halo groups; HET is a 5- to 10-membered aromatic, partially aromatic or non-aromatic mono- or bicyclic ring, containing 1-4 heteroatoms selected from O, S and N(R¹²), and optionally substituted with 1-2 oxo groups; and R¹² is selected from the group consisting of: hydrogen and C₁₋₄alkyl, optionally substituted with 1-3 halo groups.

Optionally, the Granzyme B inhibitor used herein may be selected from one of the examples detailed herein, which includes but is not limited to one or more of the following:

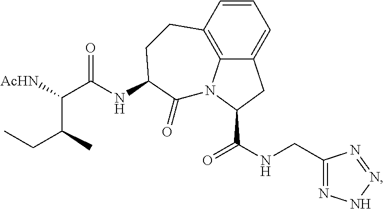 [SMILES: CC[C@H](C)[C@H](NC(C)=O)C(=O)N[C@H]1CCc2cccc3c2N(C1=O)[C@H](C(=O)NCc1nn[nH]n1)C3]

also referred to herein as (2S,5S)—N-((2H-tetrazol-5-yl)methyl)-5-((2S,3S)-2-acetamido-3-methylpentanamido)-4-oxo-1,2,4,5,6,7-hexahydroazepino[3,2,1-hi]indole-2-carboxamide,

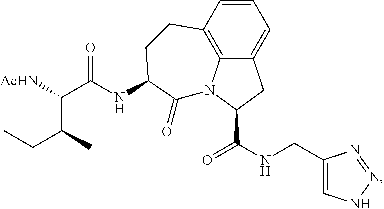 [SMILES: CC[C@H](C)[C@H](NC(C)=O)C(=O)N[C@H]1CCc2cccc3c2N(C1=O)[C@H](C(=O)NCc1c[nH]nn1)C3]

also referred to herein as (2S,5S)—N-((1H-1,2,3-triazol-4-yl)methyl)-5-((2S,3S)-2-acetamido-3-methylpentanamido)-4-oxo-1,2,4,5,6,7-hexahydroazepino[3,2,1-hi]indole-2-carboxamide,

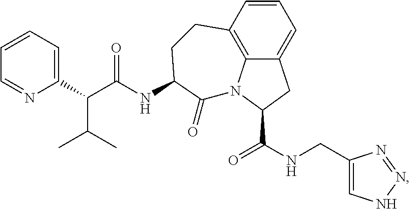 [SMILES: CC(C)[C@@H](C(=O)N[C@H]1CCc2cccc3c2N(C1=O)[C@H](C(=O)NCc1c[nH]nn1)C3)c1ccccn1]

also referred to herein as (2S,5S)—N-((1H-1,2,3-triazol-4-yl)methyl)-5-((R)-3-methyl-2-(pyridin-2-yl)butanamido)-4-oxo-1,2,4,5,6,7-hexahydroazepino[3,2,1-hi]indole-2-carboxamide,

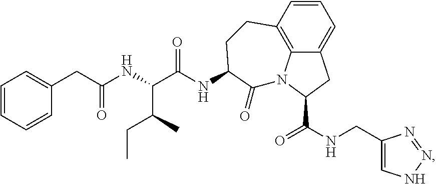 [SMILES: CC[C@H](C)[C@H](NC(=O)Cc1ccccc1)C(=O)N[C@H]1CCc2cccc3c2N(C1=O)[C@H](C(=O)NCc1c[nH]nn1)C3]

also referred to herein as (2S,5S)—N-((1H-1,2,3-triazol-4-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide,

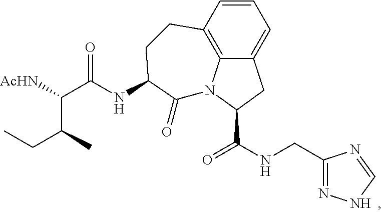 [SMILES: CC[C@H](C)[C@H](NC(C)=O)C(=O)N[C@H]1CCc2cccc3c2N(C1=O)[C@H](C(=O)NCc1nc[nH]n1)C3]

also referred to herein as (2S,5S)—N-((1H-1,2,4-triazol-3-yl)methyl)-5-((2S,3S)-2-acetamido-3-methylpentanamido)-4-oxo-1,2,4,5,6,7-hexahydroazepino[3,2,1-hi]indole-2-carboxamide,

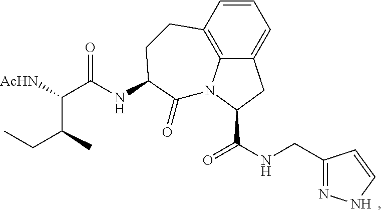 [SMILES: CC[C@H](C)[C@H](NC(C)=O)C(=O)N[C@H]1CCc2cccc3c2N(C1=O)[C@H](C(=O)NCc1cc[nH]n1)C3]

also referred to herein as (2S,5S)—N-((1H-pyrazol-3-yl)methyl)-5-((2S,3S)-2-acetamido-3-methylpentanamido)-4-oxo-1,2,4,5,6,7-hexahydroazepino[3,2,1-hi]indole-2-carboxamide,

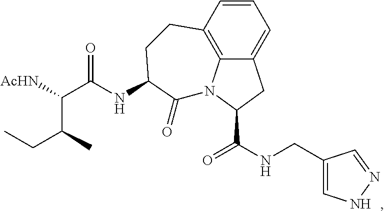 [SMILES: CC[C@H](C)[C@H](NC(C)=O)C(=O)N[C@H]1CCc2cccc3c2N(C1=O)[C@H](C(=O)NCc1cn[nH]c1)C3]

also referred to herein as (2S,5S)—N-((1H-pyrazol-4-yl)methyl)-5-((2S,3S)-2-acetamido-3-methylpentanamido)-4-oxo-1,2,4,5,6,7-hexahydroazepino[3,2,1-hi]indole-2-carboxamide,

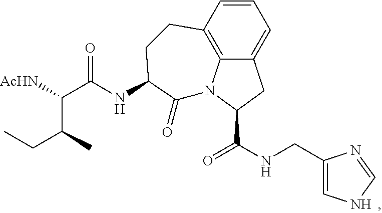 [SMILES: CC[C@H](C)[C@H](NC(C)=O)C(=O)N[C@H]1CCc2cccc3c2N(C1=O)[C@H](C(=O)NCc1c[nH]cn1)C3]

also referred to herein as (2S,5S)—N-((1H-imidazol-4-yl)methyl)-5-((2S,3S)-2-acetamido-3-methylpentanamido)-4-oxo-1,2,4,5,6,7-hexahydroazepino[3,2,1-hi]indole-2-carboxamide,

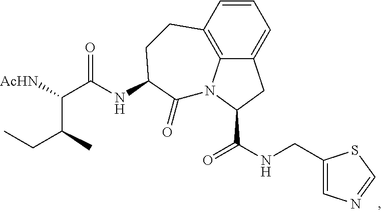 [SMILES: CC[C@H](C)[C@H](NC(C)=O)C(=O)N[C@H]1CCc2cccc3c2N(C1=O)[C@H](C(=O)NCc1cncs1)C3]

also referred to herein as (2S,5S)-5-((2S,3S)-2-acetamido-3-methylpentanamido)-4-oxo-N-(thiazol-5-ylmethyl)-1,2,4,5,6,7-hexahydroazepino[3,2,1-hi]indole-2-carboxamide,

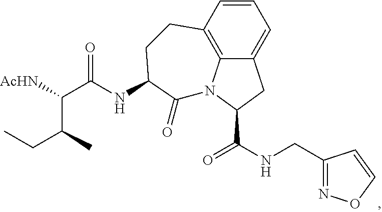 [SMILES: CC[C@H](C)[C@H](NC(C)=O)C(=O)N[C@H]1CCc2cccc3c2N(C1=O)[C@H](C(=O)NCc1ccon1)C3]

also referred to herein as (2S,5S)-5-((2S,3S)-2-acetamido-3-methylpentanamido)-N-(isoxazol-3-ylmethyl)-4-oxo-1,2,4,5,6,7-hexahydroazepino[3,2,1-hi]indole-2-carboxamide,

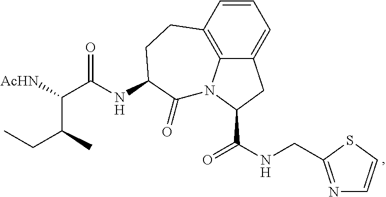 [SMILES: CC[C@H](C)[C@H](NC(C)=O)C(=O)N[C@H]1CCc2cccc3c2N(C1=O)[C@H](C(=O)NCc1nccs1)C3]

also referred to herein as (2S,5S)-5-((2S,3S)-2-acetamido-3-methylpentanamido)-4-oxo-N-(thiazol-2-ylmethyl)-1,2,4,5,6,7-hexahydroazepino[3,2,1-hi]indole-2-carboxamide,

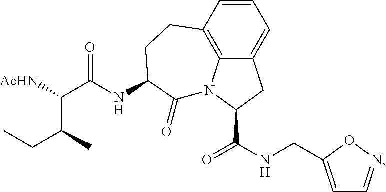 [SMILES: CC[C@H](C)[C@H](NC(C)=O)C(=O)N[C@H]1CCc2cccc3c2N(C1=O)[C@H](C(=O)NCc1ccno1)C3]

also referred to herein as (2S,5S)-5-((2S,3S)-2-acetamido-3-methylpentanamido)-N-(isoxazol-5-ylmethyl)-4-oxo-1,2,4,5,6,7-hexahydroazepino[3,2,1-hi]indole-2-carboxamide,

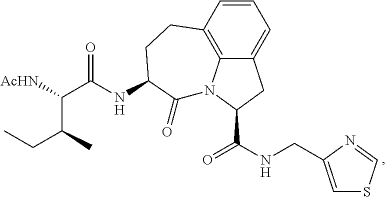 [SMILES: CC[C@H](C)[C@H](NC(C)=O)C(=O)N[C@H]1CCc2cccc3c2N(C1=O)[C@H](C(=O)NCc1cscn1)C3]

also referred to herein as (2S,5S)-5-((2S,3S)-2-acetamido-3-methylpentanamido)-4-oxo-N-(thiazol-4-ylmethyl)-1,2,4,5,6,7-hexahydroazepino[3,2,1-hi]indole-2-carboxamide,

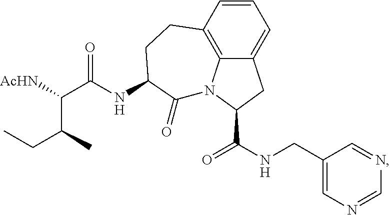 [SMILES: CC[C@H](C)[C@H](NC(C)=O)C(=O)N[C@H]1CCc2cccc3c2N(C1=O)[C@H](C(=O)NCc1cncnc1)C3]

also referred to herein as (2S,5S)-5-((2S,3S)-2-acetamido-3-methylpentanamido)-4-oxo-N-(pyrimidin-5-ylmethyl)-1,2,4,5,6,7-hexahydroazepino[3,2,1-hi]indole-2-carboxamide,

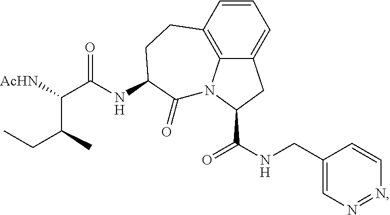 [SMILES: CC[C@H](C)[C@H](NC(C)=O)C(=O)N[C@H]1CCc2cccc3c2N(C1=O)[C@H](C(=O)NCc1ccnnc1)C3]

also referred to herein as (2S,5S)-5-((2S,3S)-2-acetamido-3-methylpentanamido)-4-oxo-N-(pyridazin-4-ylmethyl)-1,2,4,5,6,7-hexahydroazepino[3,2,1-hi]indole-2-carboxamide,

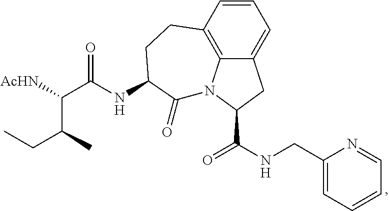 [SMILES: CC[C@H](C)[C@H](NC(C)=O)C(=O)N[C@H]1CCc2cccc3c2N(C1=O)[C@H](C(=O)NCc1ccccn1)C3]

also referred to herein as (2S,5S)-5-((2S,3S)-2-acetamido-3-methylpentanamido)-4-oxo-N-(pyridin-2-ylmethyl)-1,2,4,5,6,7-hexahydroazepino[3,2,1-hi]indole-2-carboxamide,

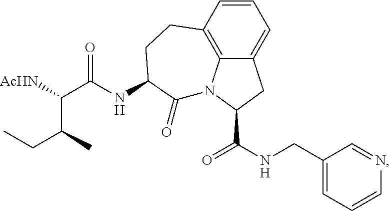 [SMILES: CC[C@H](C)[C@H](NC(C)=O)C(=O)N[C@H]1CCc2cccc3c2N(C1=O)[C@H](C(=O)NCc1cccnc1)C3]

also referred to herein as (2S,5S)-5-((2S,3S)-2-acetamido-3-methylpentanamido)-4-oxo-N-(pyridin-3-ylmethyl)-1,2,4,5,6,7-hexahydroazepino[3,2,1-hi]indole-2-carboxamide,

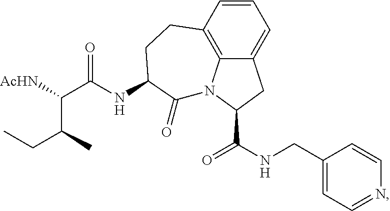 [SMILES: CC[C@H](C)[C@H](NC(C)=O)C(=O)N[C@H]1CCc2cccc3c2N(C1=O)[C@H](C(=O)NCc1ccncc1)C3]

also referred to herein as (2S,5S)-5-((2S,3S)-2-acetamido-3-methylpentanamido)-4-oxo-N-(pyridin-4-ylmethyl)-1,2,4,5,6,7-hexahydroazepino[3,2,1-hi]indole-2-carboxamide,

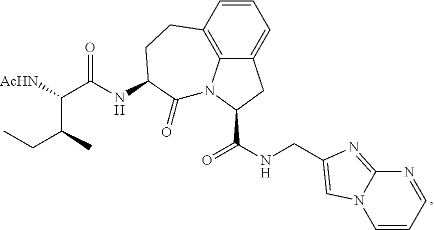 [SMILES: CC[C@H](C)[C@H](NC(C)=O)C(=O)N[C@H]1CCc2cccc3c2N(C1=O)[C@H](C(=O)NCc1cn2cccnc2n1)C3]

also referred to herein as (2S,5S)-5-((2S,3S)-2-acetamido-3-methylpentanamido)-N-(imidazo[1,2-a]pyrimidin-2-ylmethyl)-4-oxo-1,2,4,5,6,7-hexahydroazepino[3,2,1-hi]indole-2-carboxamide,

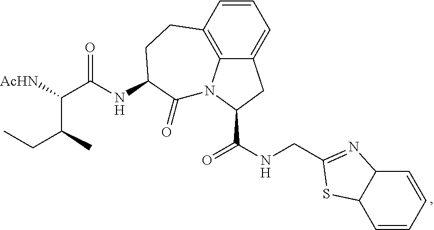 [SMILES: CC[C@H](C)[C@H](NC(C)=O)C(=O)N[C@H]1CCc2cccc3c2N(C1=O)[C@H](C(=O)NCC1=NC2C=CC=CC2S1)C3]

also referred to herein as (2S,5S)-5-((2S,3S)-2-acetamido-3-methylpentanamido)-N-((3a,7a-dihydrobenzo[d]thiazol-2-yl)methyl)-4-oxo-1,2,4,5,6,7-hexahydroazepino[3,2,1-hi]indole-2-carboxamide,

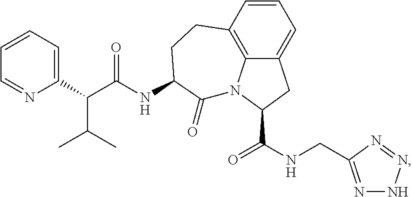 [SMILES: CC(C)[C@@H](C(=O)N[C@H]1CCc2cccc3c2N(C1=O)[C@H](C(=O)NCc1nn[nH]n1)C3)c1ccccn1]

also referred to herein as (2S,5S)—N-((2H-tetrazol-5-yl)methyl)-5-((R)-3-methyl-2-(pyridin-2-yl)butanamido)-4-oxo-1,2,4,5,6,7-hexahydroazepino[3,2,1-hi]indole-2-carboxamide,

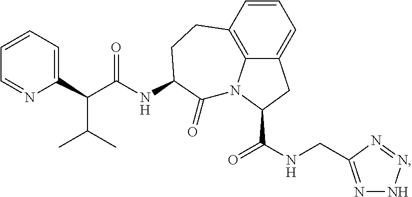 [SMILES: CC(C)[C@H](C(=O)N[C@H]1CCc2cccc3c2N(C1=O)[C@H](C(=O)NCc1nn[nH]n1)C3)c1ccccn1]

also referred to herein as (2S,5S)—N-((2H-tetrazol-5-yl)methyl)-5-((S)-3-methyl-2-(pyridin-2-yl)butanamido)-4-oxo-1,2,4,5,6,7-hexahydroazepino[3,2,1-hi]indole-2-carboxamide,

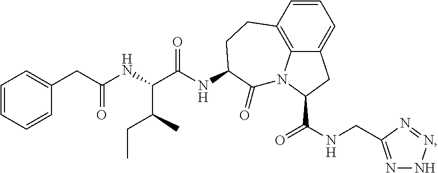 [SMILES: CC[C@H](C)[C@H](NC(=O)Cc1ccccc1)C(=O)N[C@H]1CCc2cccc3c2N(C1=O)[C@H](C(=O)NCc1nn[nH]n1)C3]

also referred to herein as (2S,5S)—N-((2H-tetrazol-5-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide,

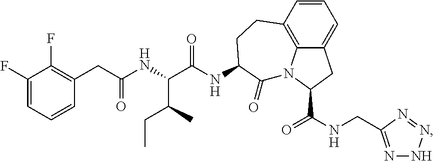 [SMILES: CC[C@H](C)[C@H](NC(=O)Cc1cccc(F)c1F)C(=O)N[C@H]1CCc2cccc3c2N(C1=O)[C@H](C(=O)NCc1nn[nH]n1)C3]

also referred to herein as (2S,5S)—N-((2H-tetrazol-5-yl)methyl)-5-((2S,3S)-2-(2-(2,3-difluorophenyl)acetamido)-3-methylpentanamido)-4-oxo-1,2,4,5,6,7-hexahydroazepino[3,2,1-hi]indole-2-carboxamide,

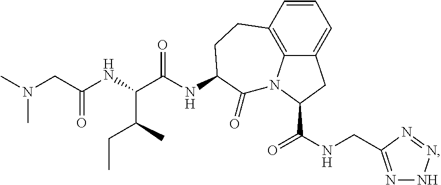 [SMILES: CC[C@H](C)[C@H](NC(=O)CN(C)C)C(=O)N[C@H]1CCc2cccc3c2N(C1=O)[C@H](C(=O)NCc1nn[nH]n1)C3]

also referred to herein as (2S,5S)—N-((2H-tetrazol-5-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide,

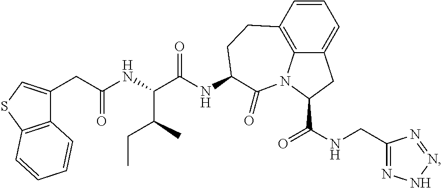 [SMILES: CC[C@H](C)[C@H](NC(=O)Cc1csc2ccccc12)C(=O)N[C@H]1CCc2cccc3c2N(C1=O)[C@H](C(=O)NCc1nn[nH]n1)C3]

also referred to herein as (2S,5S)—N-((2H-tetrazol-5-yl)methyl)-5-((2S,3S)-2-(2-benzo[b]thiophen-3-yl)acetamido)-3-methylpentanamido)-4-oxo-1,2,4,5,6,7-hexahydroazepino[3,2,1-hi]indole-2-carboxamide,

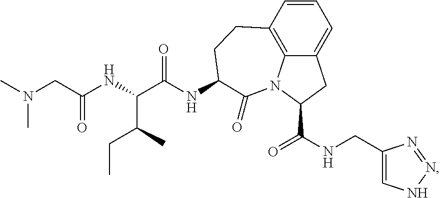 [SMILES: CC[C@H](C)[C@H](NC(=O)CN(C)C)C(=O)N[C@H]1CCc2cccc3c2N(C1=O)[C@H](C(=O)NCc1c[nH]nn1)C3]

also referred to herein as (2S,5S)—N-((1H-1,2,3-triazol-4-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide,

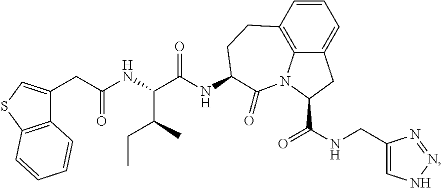 [SMILES: CC[C@H](C)[C@H](NC(=O)Cc1csc2ccccc12)C(=O)N[C@H]1CCc2cccc3c2N(C1=O)[C@H](C(=O)NCc1c[nH]nn1)C3]

also referred to herein as (2S,5S)—N-((1H-1,2,3-triazol-4-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide,

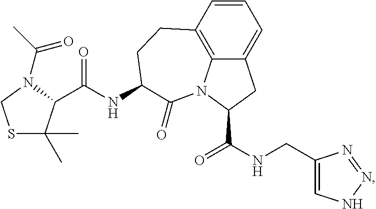 [SMILES: CC(=O)N1CSC(C)(C)[C@H]1C(=O)N[C@H]1CCc2cccc3c2N(C1=O)[C@H](C(=O)NCc1c[nH]nn1)C3]

also referred to herein as (R)—N-((2S,5S)-2-((1H-1,2,3-triazol-4-yl)methylcarbamoyl)-4-oxo-1,2,4,5,6,7-hexahydroazepino[3,2,1-hi]indol-5-yl)-3-acetyl-5,5-dimethylthiazolidine-4-carboxamide,

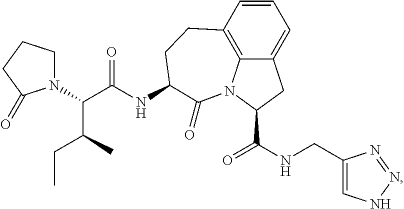 [SMILES: CC[C@H](C)[C@@H](C(=O)N[C@H]1CCc2cccc3c2N(C1=O)[C@H](C(=O)NCc1c[nH]nn1)C3)N1CCCC1=O]

also referred to herein as (2S,5S)—N-((1H-1,2,3-triazol-4-yl)methyl)-5-((2S,3S)-3-methyl-2-(2-oxopyrrolidin-1-yl)pentanamido)-4-oxo-1,2,4,5,6,7-hexahydroazepino[3,2,1-hi]indole-2-carboxamide,

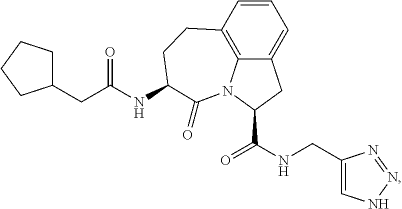 [SMILES: O=C(CC1CCCC1)N[C@H]1CCc2cccc3c2N(C1=O)[C@H](C(=O)NCc1c[nH]nn1)C3]

also referred to herein as (2S,5S)—N-((1H-1,2,3-triazol-4-yl)methyl)-5-(2-cyclopentylacetamido)-4-oxo-1,2,4,5,6,7-hexahydroazepino[3,2,1-hi]indole-2-carboxamide,

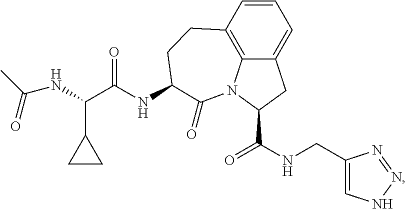 [SMILES: CC(=O)N[C@H](C(=O)N[C@H]1CCc2cccc3c2N(C1=O)[C@H](C(=O)NCc1c[nH]nn1)C3)C1CC1]

also referred to herein as (2S,5S)—N-((1H-1,2,3-triazol-4-yl)methyl)-5-((S)-2-acetamido-2-cyclopropylacetamido)-4-oxo-1,2,4,5,6,7-hexahydroazepino[3,2,1-hi]indole-2-carboxamide,

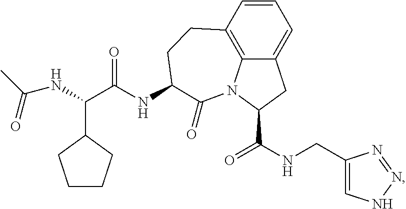 [SMILES: CC(=O)N[C@H](C(=O)N[C@H]1CCc2cccc3c2N(C1=O)[C@H](C(=O)NCc1c[nH]nn1)C3)C1CCCC1]

also referred to herein as (2S,5S)—N-((1H-1,2,3-triazol-4-yl)methyl)-5-((S)-2-acetamido-2-cyclopentylacetamido)-4-oxo-1,2,4,5,6,7-hexahydroazepino[3,2,1-hi]indole-2-carboxamide, or salt or solvate thereof.

Optionally, the Granzyme B inhibitor used herein may be selected from one of the examples detailed herein, which includes but is not limited to one or more of the following:

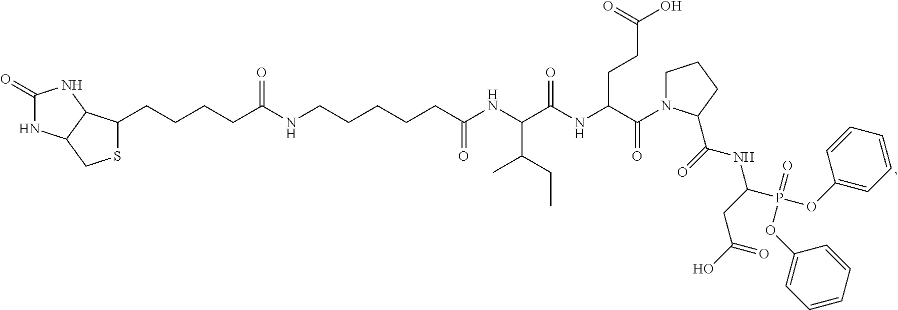 [SMILES: CCC(C)C(NC(=O)CCCCCNC(=O)CCCCC1SCC2NC(=O)NC21)C(=O)NC(CCC(=O)O)C(=O)N1CCCC1C(=O)NC(CC(=O)O)P(=O)(Oc1ccccc1)Oc1ccccc1]

also referred to herein as Bio-x-IEPD^{P}-(OPh)₂,

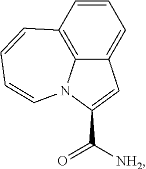

also referred to herein as azepino[3,2,1-hi]indole-2-carboxamide,

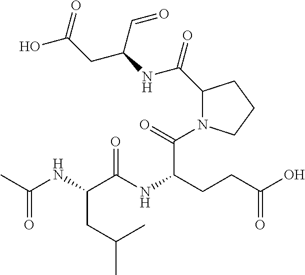 [SMILES: CC(=O)N[C@@H](CC(C)C)C(=O)N[C@@H](CCC(=O)O)C(=O)N1CCCC1C(=O)N[C@H](C=O)CC(=O)O]

also referred to herein as (4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid,

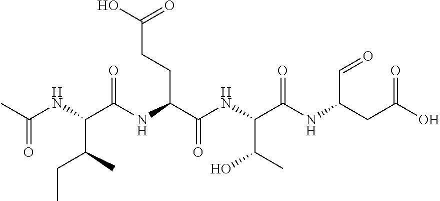 [SMILES: CC[C@H](C)[C@H](NC(C)=O)C(=O)N[C@@H](CCC(=O)O)C(=O)N[C@H](C(=O)N[C@H](C=O)CC(=O)O)[C@H](C)O]

also referred to herein as (4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid,

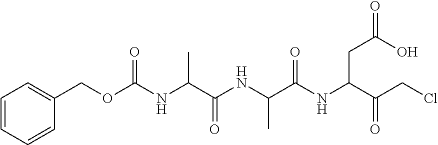 [SMILES: CC(NC(=O)OCc1ccccc1)C(=O)NC(C)C(=O)NC(CC(=O)O)C(=O)CCl]

also referred to herein as 5-chloro-4-oxo-3-[2-[2-(phenylmethoxycarbonylamino) propanoylamino]propanoylamino]pentanoic acid,

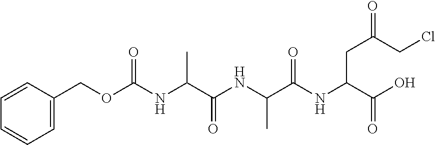 [SMILES: CC(NC(=O)OCc1ccccc1)C(=O)NC(C)C(=O)NC(CC(=O)CCl)C(=O)O]

also referred to herein as 5-chloro-4-oxo-2-[2-[2-(phenylmethoxycarbonylamino) propanoylamino]propanoylamino]pentanoic acid, or a salt or solvate thereof.

Optionally, the Granzyme B inhibitors used herein is selected from the following:

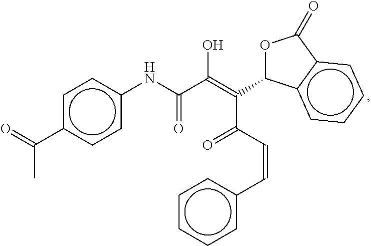

also referred to herein as ZINC05723764 and NCI 644752,

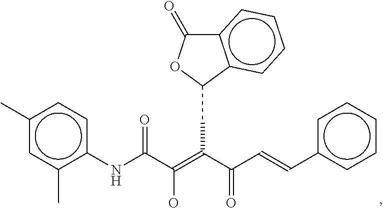

also referred to herein as ZINC05723787 and NCI 644777,

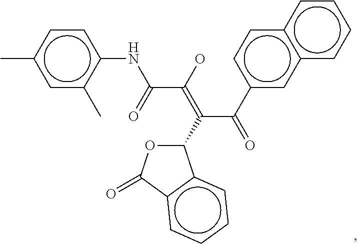

also referred to herein as ZINC05316154 and NCI 641248,

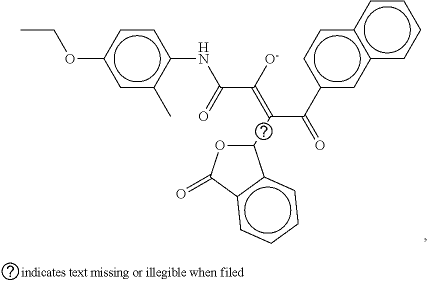

also referred to herein as ZINC05723499 and NCI 641235,

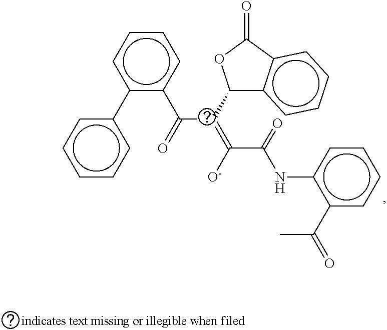

also referred to herein as ZINC05723646 and NCI 642017,

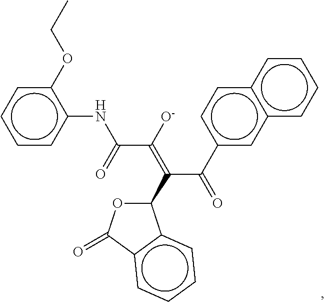

also referred to herein as ZINC05398428 and NCI 641230,

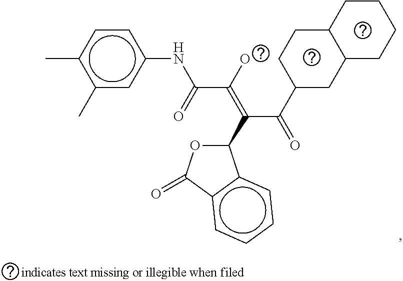

also referred to herein as ZINC5723503 and NCI 641236,

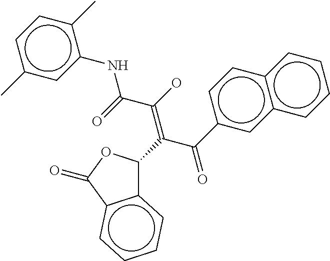

also referred to herein as ZINC05723446 and and NCI 640985,

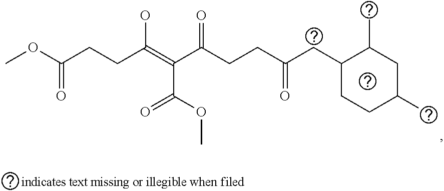

also referred to herein as ZINC05317216 and NCI 618792,

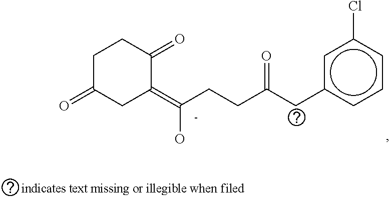

also referred to herein as ZINC05315460 and NCI 630295,

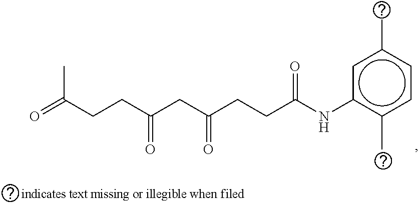

also referred to herein as ZINC05316859 and NCI 618802, and

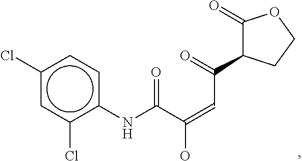

also referred to herein as ZINC05605947 and NCI 623744, or a salt or solvate thereof.

Optionally, the Granzyme B inhibitor used herein is:

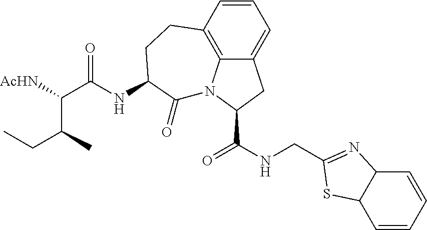 [SMILES: CC[C@H](C)[C@H](NC(C)=O)C(=O)N[C@H]1CCc2cccc3c2N(C1=O)[C@H](C(=O)NCC1=NC2C=CC=CC2S1)C3]

or a salt or solvate thereof.

Optionally, the Granzyme B inhibitor used herein is:

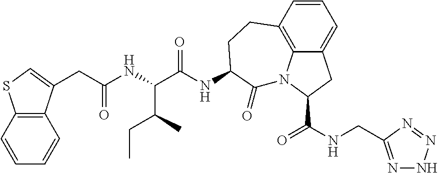 [SMILES: CC[C@H](C)[C@H](NC(=O)Cc1csc2ccccc12)C(=O)N[C@H]1CCc2cccc3c2N(C1=O)[C@H](C(=O)NCc1nn[nH]n1)C3]

or a salt or solvate thereof.

Optionally, the Granzyme B inhibitor used herein is:

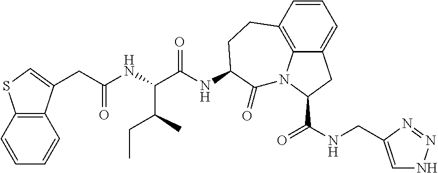 [SMILES: CC[C@H](C)[C@H](NC(=O)Cc1csc2ccccc12)C(=O)N[C@H]1CCc2cccc3c2N(C1=O)[C@H](C(=O)NCc1c[nH]nn1)C3]

or a salt or solvate thereof.

A Granzyme B inhibitor for use in the methods, compositions, and uses of the invention may also be a synthetic inhibitor such as, for example, an isocoumarin, a peptide chloromethyl ketone, or a peptide phosphonate (see, e.g. Kam et al., 2000).

Optionally, the Granzyme B inhibitor used herein is one or more of:

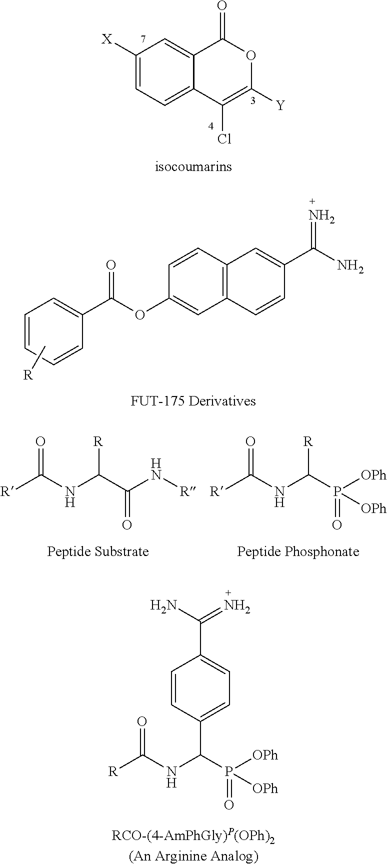

Isocoumarin derivatives (upper left): 3,4-dichloroisocoumarin, DCI, X=H, Y=Cl; 7-amino-4-chloro-3-(3-isothiureidopropoxy)isocoumarin, X=NH₂, Y=O(CH₂)₃—SC(═NH⁺₂)NH₂; 4-chloro-3-ethoxy-7-guanidinoisocoumarin, X=NHC(═NH⁺₂)NH₂, Y=OCH₂CH₃. FUT-175 analogs (upper right). Bottom line: structures of a peptide substrate, a peptide phosphonate and a 4-amidinophenylglycine phosphonate [(4-AmPhGly)^{P}(OPh)₂] derivative. The latter is an arginine analog.

### III. Pharmaceutical Compositions

Many Granzyme B inhibitors are water-soluble and may be formed as salts. In such cases, compositions of Granzyme B inhibitors may comprise a physiologically acceptable salt, which are known to a person of skill in the art. Preparations will typically comprise one or more carriers acceptable for the mode of administration of the preparation, be it by topical administration, lavage, epidermal administration, sub-epidermal administration, dermal administration, sub-dermal administration, sub-cutaneous administration, systemic administration, injection, inhalation, oral, or other modes suitable for the selected treatment. Suitable carriers are those known in the art for use in such modes of administration.

Suitable compositions may be formulated by means known in the art and their mode of administration and dose determined by a person of skill in the art. For parenteral administration, compound may be dissolved in sterile water or saline or a pharmaceutically acceptable vehicle used for administration of non-water soluble compounds such as those used for vitamin K. For enteral administration, compound may be administered in a tablet, capsule, or dissolved in liquid form. The tablet or capsule may be enteric coated, or in a formulation for sustained release. Many suitable formulations are known including, polymeric or protein microparticles encapsulating a compound to be released, ointments, pastes, gels, hydrogels, foams, creams, powders, lotions, oils, semi-solids, soaps, medicated soaps, shampoos, medicated shampoos, sprays, films, or solutions which can be used topically or locally to administer a compound. A sustained release patch or implant may be employed to provide release over a prolonged period of time. Many techniques known to one of skill in the an are described in Remington: the Science & Practice of Pharmacy by Alfonso Gennaro, 20^{th} ed., Williams & Wilkins, (2000). Formulations may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful delivery systems for modulatory compounds include ethylene-vinyl acetate copolymer particles, osmotic pumps, implantable infusion systems, and liposomes. Formulations may contain excipients, for example, lactose, or may be aqueous solutions containing, for example, polyoxyethylene-9-lauryl ether, glycocholate and deoxycholate, or may be oily solutions for administration in the form of drops, or as a gel.

Compositions containing Granzyme B inhibitors may also include penetrating agents. Penetrating agents may improve the ability of the Granzyme B inhibitors to be delivered to deeper layers of the skin. Penetrating agents that may be used are known to a person of skill in the art and include, but are not limited to, hyaluronic acid, insulin, liposome, or the like, as well as L-arginine or the arginine-containing amino acids.

Compounds or compositions of Granzyme B inhibitors may be administered alone or in conjunction with other wound treatments, such as wound preparations, wound coverings, and closure devices.

Optionally, the Granzyme B inhibitor is formulated for topical administration. For example, the formulations for topical administration of a Granzyme B inhibitor may assume any of a variety of dosage forms, including solutions, suspensions, ointments, and solid inserts. Examples are creams, lotions, gels, ointments, suppositories, sprays, foams, liniments, aerosols, buccal and sublingual tablets, various passive and active topical devices for absorption through the skin and mucous membranes, including transdermal applications, and the like.

The Granzyme B inhibitor may be formulated for co-administration with another wound treatment. The another wound treatment may be selected from one or more of the following: a topical antimicrobial; a cleanser; a wound gel; a collagen; an elastin; a tissue growth promoter; an enzymatic debriding preparation; an antifungal; an anti-inflammatory; a barrier; a moisturizer; and a sealant. Optionally, the another wound treatment may be selected from one or more of the following: a wound covering, a wound filler, and an implant. Optionally, the another wound treatment may be selected from one or more of the following: absorptive dressings; alginate dressings: foam dressings; hydrocolloid dressings; hydrofiber dressings; compression dressing and wraps; composite dressing; contact layer; wound gel impregnated gauzes; wound gel sheets; transparent films; wound fillers; dermal matrix products or tissue scaffolds; and closure devices. Optionally, the Granzyme B inhibitor is formulated for topical application in a wound covering, a wound filler, or an implant. Optionally, the Granzyme B inhibitor is formulated for impregnation in a wound covering, a wound filler or an implant. The subject contemplated herein may be a mammal. Further, the subject contemplated herein may be a human.

Optionally, the Granzyme B inhibitor may be formulated for topical administration. Optionally, the Granzyme B inhibitor may be formulated for co-administration with another wound treatment. Optionally, the wound treatment may be selected from one or more of: a topical antimicrobial; a cleanser; a wound gel; a collagen; a elastin; a tissue growth promoter; an enzymatic debriding preparation; an antifungal; an anti-inflammatory; a barrier; a moisturizer; and a sealant. Optionally, another wound treatment may be selected from one or more of: a wound covering, a wound filler and an implant. Optionally, the another wound treatment may be selected from one or more of: absorptive dressings; alginate dressings; foam dressings; hydrocolloid dressings; hydrofiber dressings; compression dressing and wraps; composite dressing; contact layer; wound gel impregnated gauzes; wound gel sheets; transparent films; wound fillers; dermal matrix products or tissue scaffolds; and closure devices. Optionally, the Granzyme B inhibitor may be formulated for topical application in a wound covering, a wound filler, or an implant. Optionally, the Granzyme B inhibitor may be formulated for impregnation in a wound covering, a wound filler or an implant. Optionally, the use may involve a subject that is a mammal; optionally, the use may involve a subject that is a human.

### IV. Animal Models and Screening Methods

In another aspect, a model for studying age-related wound repair is disclosed. The model comprises an apolipoprotein E-knock out mouse maintained on a high-fat feed diet, wherein the high-fat feed diet is sufficient to result in xanthomatotic skin lesions on the mouse, and wherein the high-fat feed diet is sufficient to result in premature aging of non-xanthomatous regions of the skin. In skin areas that do not contain xanthomas, these mice also develop evidence of skin aging in the form of reduced skin thickness, reduced collagen, and reduced elasticity when fed a high-fat diet.

In another aspect, a model for studying Granzyme B protein expression in vivo is disclosed. The model comprises an apolipoprotein E-knock out mouse maintained on a high-fat feed diet, wherein the high-fat feed diet is sufficient to result in xanthomatotic skin lesions on the mouse, and wherein the skin lesions express Granzyme B. Granzyme B is abundant in the epidermal-dermal junction, an area that is prone to damage and separation as skin ages and during skin ulcer formation. This area also contains a large amount of the Granzyme B substrate decorin.

In another aspect, a model for studying premature aging in skin is disclosed. The model comprises an apolipoprotein E-knock out mouse maintained on a high-fat feed diet, wherein the high-fat feed diet is sufficient to result in premature aging of the skin.

In another aspect, a model for screening compounds involved in repairing wounds is disclosed. The method involves maintaining an apolipoprotein E-knock out mouse on a high-fat feed diet, wherein the high-fat feed diet is sufficient to result in accelerated age-related changes in the skin, thinning, and/or skin lesions on the mouse; administering a compound to the skin lesions on the mouse; and monitoring the skin lesions on the mouse. The monitoring contemplated herein includes any biological sign of repair of a skin lesion. Examples of modes by which repair can be monitored include, but are not limited to the following: monitoring the presence or absence of newly formed tissue, and monitoring the width and/or size of the lesion, hair loss and/or restoration on the lesion. Other methods that can be employed include, but are not limited to, the following: monitoring the skin surface temperature, measuring transepidermal water loss, monitoring the presence or absence of ECM abnormalities, elastosis, collagen morphology, collagen density, the presence of decorin, and restoration of proper skin thickness. Additionally, skin-stress studies could be employed. Further, and serving as an example, decorin is reduced in areas of wound healing and fibrosis.

In another aspect, a method of screening compounds involved in repairing wounds is disclosed. The method involves maintaining an apolipoprotein E-knock out mouse on a high-fat feed diet, wherein the high-fat feed diet is sufficient to result in skin lesions on the mouse, and wherein the skin lesions express Granzyme B; administering a compound to the skin lesions on the mouse; and monitoring the skin lesions on the mouse.

In another aspect, a method of screening compounds involved in inhibiting or reducing skin lesions is disclosed. The method involves maintaining an apolipoprotein E-knock out mouse on a high-fat feed diet, wherein the high-fat feed diet is sufficient to result in skin lesions on the mouse when a compound is not administered to the mouse; administering the compound to the mouse; and monitoring the skin lesions on the mouse.

In another aspect, a method of screening compounds involved in inhibiting or reducing skin lesions is disclosed. The method involves maintaining an apolipoprotein E-knock out mouse on a high-fat feed diet, wherein the high-fat feed diet is sufficient to result in skin lesions on the mouse when a compound is not administered to the mouse, and wherein the skin lesions express Granzyme B; administering the compound to the skin lesions on the mouse; and monitoring the skin lesions on the mouse.

In another aspect, the present invention provides methods for identifying a compound useful for promoting chronic wound healing. The methods include providing an indicator composition comprising decorin and Granzyme B; contacting the indicator composition with each of a plurality of test compounds; and determining the effect of each of the plurality of test compounds on the cleavage of decorin, and selecting a compound that inhibits the cleavage of decorin in the indicator composition, thereby identifying a compound useful for promoting chronic wound healing.

The methods may further comprise determining the effect of the compound of collagen density and organization, the release of sequestered cytokine, e.g. TGF-β, the cleavage of an extracellular matrix protein, e.g., an extracellular proteoglycan, such as biglycan, and/or the tensile strength of skin.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al., (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

The indicator composition can be a cell that expresses the Granzyme b and/or decorin protein, for example, a cell that naturally expresses or has been engineered to express the protein(s) by introducing into the cell an expression vector encoding the protein(s).

Alternatively, the indicator composition can be a cell-free composition that includes the protein(s) (e.g., a cell extract or a composition that includes e.g., either purified natural or recombinant protein).

For example, an indicator cell can be transfected with an expression vector, incubated in the presence and in the absence of a test compound, and the effect of the compound on the expression of the molecule or on a biological response can be determined.

A variety of cell types are suitable for use as an indicator cell in the screening assay. Cells for use in the subject assays include eukaryotic cells. For example, in one embodiment, a cell is a vertebrate cell, e.g., an avian cell or a mammalian cell (e.g., a murine cell, or a human cell).

Recombinant expression vectors that can be used for expression of, e.g. decorin, are known in the art. For example, the cDNA is first introduced into a recombinant expression vector using standard molecular biology techniques. A cDNA can be obtained, for example, by amplification using the polymerase chain reaction (PCR) or by screening an appropriate cDNA library. The nucleotide sequences of cDNAs for or a molecule in a signal transduction pathway involving (e.g., human, murine and bacterial) are known in the art and can be used for the design of PCR primers that allow for amplification of a cDNA by standard PCR methods or for the design of a hybridization probe that can be used to screen a cDNA library using standard hybridization methods.

In another embodiment, the indicator composition is a cell free composition. Protein expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

Once a test compound is identified that directly or indirectly modulates, e.g., decorin cleavage by one of the variety of methods described hereinbefore, the selected test compound (or “compound of interest”) can then be further evaluated for its effect on cells, for example by contacting the compound of interest with cells either in vivo (e.g., by administering the compound of interest to an organism) or ex vivo (e.g., by isolating cells from an organism and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

In another aspect, the invention pertains to a combination of two or more of the assays described herein.

Moreover, a compound identified as described herein (e.g., an antisense nucleic acid molecule, or a specific antibody, or a small molecule) can be used in an animal model to determine the efficacy, toxicity, or side effects of treatment with such a modulator. Alternatively, a modulator identified as described herein can be used in an animal model to determine the mechanism of action of such a modulator.

The instant invention also pertains to compounds identified in the subject screening assays.

### EXAMPLES

### Abbreviations Used Herein

CTL, cytotoxic lymphocytes; DCI, 3,4-dichloroisocoumarin; DMSO, dimethyl sulfoxide; ECM, extracellular matrix; Erk, extracellular signal-regulated kinase; GAG, glycosaminoglycan; Granzyme B, Granzyme B; HCASMC, human coronary artery smooth muscle cells; NK, natural killer cell; LAP, latency associated peptide; LLC, large latent TGF-β complex; LTBP, latent TGF-β binding protein; MMP, matrix metalloproteinase; MT-MMP1, membrane type-matrix metalloproteinase 1; SLC, small latent TGF-β complex; TGF-β, transforming growth factor beta.

### Example 1

### Granzyme B Cleaves Extracellular Matrix Proteins

Methods. For in vitro extracellular matrix cleavage assays, cells were grown to confluency and lysed, leaving the intact ECM on the plate. ECM was then biotinylated. Plates were then washed with PBS and incubated at 37° C. with Granzyme B and/or with the Granzyme B inhibitor, 3,4-dichloroisocoumarin (DCI), for 4 and 24 hours at room temperature. Supernatant was then collected and assessed for cleavage fragments. Fragments were determined by Western blotting or N-terminal sequencing. Confirmation of cleavage was performed subsequently with purified substrate.

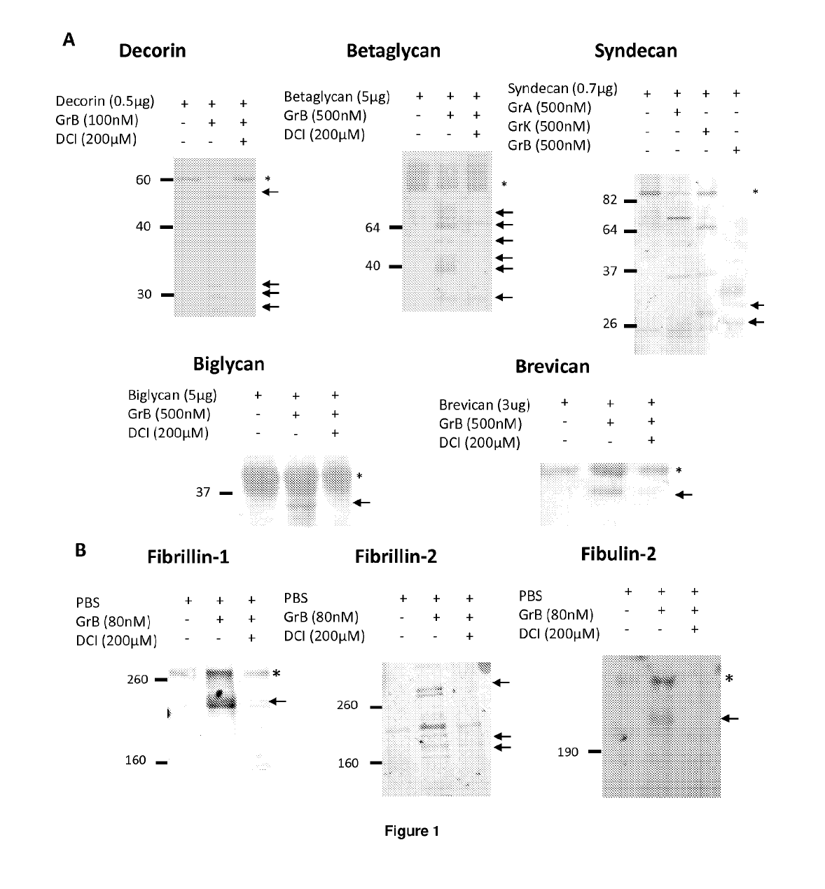

Results: In order to identify extracellular Granzyme B substrates, recombinant decorin, biglycan, betaglycan, syndecan, and brevican were incubated with purified Granzyme B for 24 hours. Reactions were stopped with SDS-PAGE loading buffer, run on an SDS-PAGE gel and imaged by Ponceau staining of a nitrocellulose membrane. As shown in FIG. 1A, Granzyme B cleaves recombinant decorin, biglycan, betaglycan, syndecan, and brevican.

In order to determine if Granzyme B also cleaves smooth muscle cell- (SMC-)derived ECM, following 5-7 days of serum starvation for ECM synthesis, human coronary artery smooth muscle cells (HCASMCs) were removed from 6 well plates using ammonium hydroxide. Granzyme B was incubated on ECM for 24 hours and supernatants were western blotted for fibrillin-1, fibrillin-2 or fibulin-2 (FIG. 1B).

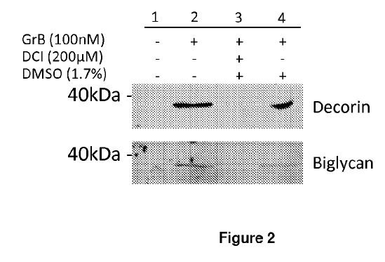

FIG. 2 shows that Granzyme B also cleaves smooth muscle cell-derived decorin and biglycan. HCASMCs were incubated at confluency for adequate ECM synthesis. Cells were removed, Granzyme B was incubated with the ECM, and decorin and biglycan cleavage fragments were detected by western immunoblotting.

### Example 2

### Granzyme B Cleaves Proteoglycans and Releases Sequestered TGF-β from Extracellular Matrix

Methods:

For ECM cleavage assays, Granzyme B and/or the inhibitor 3,4-dichloroisocoumarin (DCI), were incubated for 4 and 24 hours at room temperature, with decorin, biglycan or soluble betaglycan and visualized by Ponceau staining. Cleavage fragments were subjected to Edman degradation for cleavage site identification.

As TGF-β is sequestered by the aforementioned proteoglycans, Granzyme B was incubated with TGF-β bound proteoglycans to determine if Granzyme B cleavage resulted in the release of sequestered TGF-β. Cytokine release was assessed in supernatants using Western blotting.

To determine if the TGF-β released by Granzyme B was active, supernatants from the above release assay were incubated on human coronary artery smooth muscle cells (HCASMC) and SMAD/Erk activation was examined by Western blotting.

Results:

Granzyme B cleaved decorin, biglycan and betaglycan, with proteolysis evident at Granzyme B concentrations as low as 25 nM. Proteolysis was inhibited by DCI but not the solvent control DMSO. Edman degradation analysis determined Granzyme B cleavage sites in the PGs with P1 residues of aspartic acid, consistent with Granzyme B cleavage specificity.

In cytokine release assays, TGF-β was liberated Granzyme B-dependently from decorin, biglycan, and betaglycan, after 24 h of incubation. TGF-β was not released in the absence of Granzyme B or when Granzyme B was inhibited by DCI, indicating release from decorin, biglycan and betaglycan was specific. In addition, the TGF-β liberated by Granzyme B remained active and induced SMAD-3 and Erk-2 phosphorylation in HCASMC, after 16 h of incubation (see below).

### Example 3

### Granzyme B Cleaves Decorin, Biglycan and Soluble Betaglycan and Releases Active Transforming Growth Factor-β

Methods:

Proteoglycan cleavage assays. The recombinant human PGs, decorin (0.5 μg, Abnova, Walnut, Calif.), biglycan and betaglycan (1.5-5 ug, R&D Systems, Minneapolis, Minn.) were incubated at room temperature for 24 h with 25-500 nM purified human Granzyme B (Axxora, San Diego, Calif.), in 50 mM Tris buffer, pH 7.4. For inhibitor studies, Granzyme B was incubated in the presence or absence of 200 μM of the serine protease inhibitor 3,4-dichloroisocoumarin (DCI; Santa Cruz Biotechnology Inc, Santa Cruz, Calif.) or inhibitor solvent control, dimethyl sulfoxide (DMSO; Sigma-Aldrich, St Louis, Mo.) for 4 h or 24 h. After incubation, proteins were denatured, separated on a 10% SDS-polyacrylamide gel and transferred to a nitrocellulose membrane. Ponceau stain (Fisher Scientific, Waltham, Mass.) was used to detect cleavage fragments.

N-Terminal Sequencing.

For Edman degradation. 2-5 μg/lane of biglycan and betaglycan were incubated with 100-500 nM Granzyme B for 24 h. Once run on a gel and transferred to a PVDF membrane, cleavage fragments were identified by Ponceau staining. The stain was removed by washes with distilled water, the membrane was dried and analyzed at the Advanced Protein Technology Center at the Hospital for Sick Kids (Toronto, ON).

TGF-β Release Assays.

TGF-β release assays were carried out using a method similar to that previously described for the MMPs (Imai et al. 1997). Briefly, decorin, biglycan and betaglycan (15 μg/mL) were coated onto 48 well tissue culture plastic plates and allowed to incubate overnight at 4° C. in PBS, pH 7.4. After blocking with 1% bovine serum albumin, 20 ng of active TGF-β1 per well (Peprotech Inc, Rocky Hill, N.J.) was added in DPBS containing calcium and magnesium (#14040, Invitrogen, Carlsbad, Calif.) for 5 h at RT. Granzyme B, with or without DCI, was then added to the wells. After 24 h, supernatants were removed, denatured, and run on a 15% SDS-PAGE gel. Once transferred to a nitrocellulose membrane and blocked with 10% skim milk, the membrane was probed using a rat anti-human TGF-β1 antibody (1:200, BD Biosciences, Franklin Lakes, N.J.) and IRDye® 800 conjugated affinity purified anti-Rat IgG (1:3000, Rockland Inc, Gilbertsville, Pa.). Bands were imaged using the Odyssey Infrared Imaging System (LI-COR Biotechnology, Lincoln, Nebr.).

Human Coronary Artery Smooth Muscle Cell TGF-β Bioavailability Assays.

For bioavailability assays, HCASMCs (Clonetics/Lonza, Walkersville, Md.) at passage 3-5 were seeded in 6 well plates in smooth muscle cell growth media (SmGM, Clonetics) +5% fetal bovine serum (FBS, Invitrogen) and grown to confluence. At this time, cells were quiesced by serum removal for 24 h, after which time 150 μl of release assay supernatants (as described above) or a 10 ng TGF-β positive control were added to the cells for 16 h. Cell lysates were assessed by SDS-PAGE/Western blotting for phosphorylated-Erk 1/2 (p-Erk1/2; 1:1000, Cell Signaling Technology, Danvers, Mass.), total Erk 1/2 (t-Erk1/2; 1:1000, Cell Signaling Technology), phosphorylated-SMAD3 (p-SMAD3; 1:2000, Epitomics, Burlingame, Calif.), total SMAD3 (t-SMAD3; 1:500, BD Biosciences) and the loading controls β-actin (1:5000, Sigma-Aldrich) or β-tubulin (1:3000, Millipore, Billerica, Mass.). Secondary IRDye® 800 conjugated antibodies (1:3000, Rockland Inc) were utilized and imaged with the Odyssey Infrared Imaging System (LI-COR Biotechnology). Densitometric analysis was conducted on the Odyssey Infrared Imaging System and displayed graphically by p-SMAD-3/β-tubulin and p-Erk/β-actin ratios.

Results:

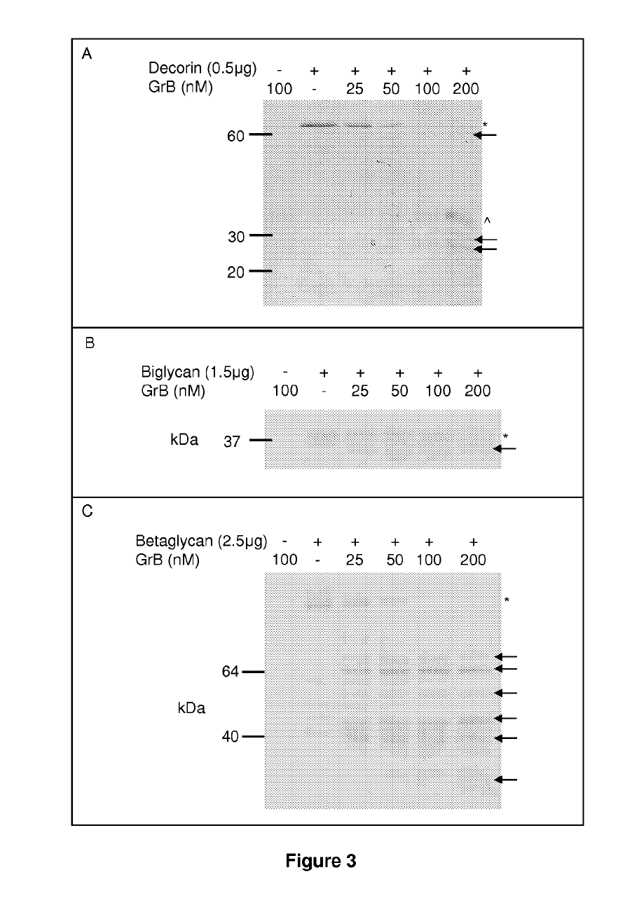

Granzyme B cleaves decorin, biglycan and betaglycan. Incubation of decorin, biglycan and betaglycan with Granzyme B resulted in the concentration-dependent generation of multiple cleavage fragments (FIG. 3a-c). Full length decorin (˜65 kDa) and 4 decorin cleavage fragments at ˜50 kDa and ˜30 kDa, were evident following Granzyme B incubation. Biglycan was identified at ˜40 kDa, with cleavage fragments evident at ˜25 kDa and 15 kDa, while incubation of recombinant soluble betaglycan (˜100 kDa) with Granzyme B resulted in multiple cleavage fragments at ˜60 kDa and 40 kDa. As all of these substrates are PGs and contain glycosaminoglycan (GAG) chains, the apparent MW of the full-length proteins and fragments may not be accurate, as glycosylation can alter movement through the gel. As such, several of the proteins and protein fragments are observed as a smear as opposed to a condensed band.

Referring to FIG. 3, dose dependent Granzyme B-mediated cleavage of decorin, biglycan and betaglycan is demonstrated therein. Increasing concentrations of Granzyme B (25, 50, 100 and 200 nM) were incubated with decorin (a), biglycan (b), and betaglycan (c) for 24 h at RT. As used in FIG. 3, the mark * denotes full-length protein, arrows indicate cleavage fragments and ̂ indicates Granzyme B.

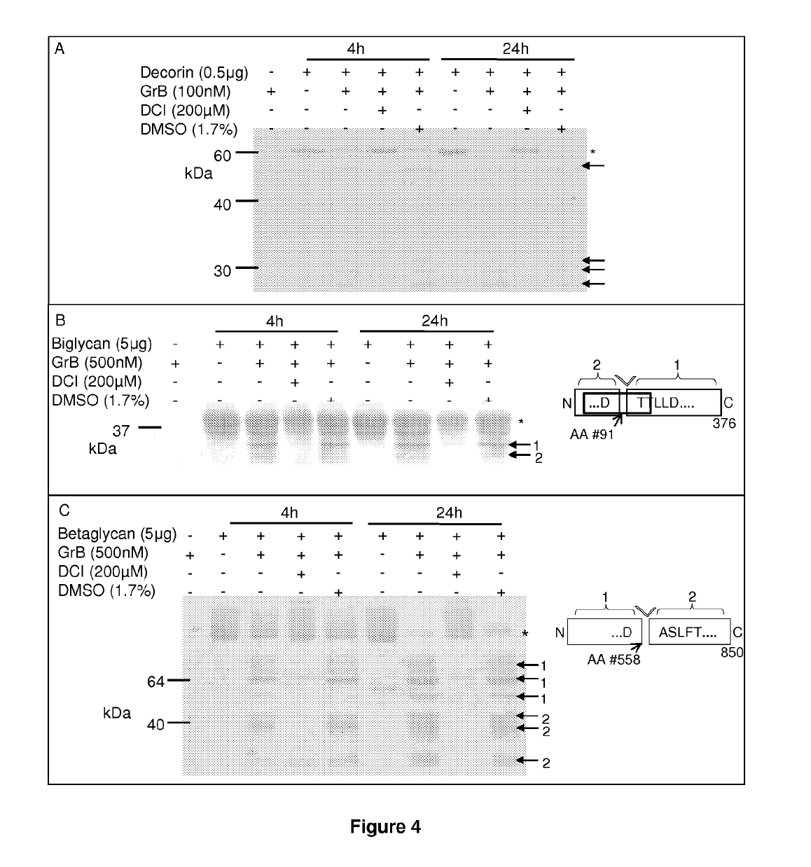

To confirm that decorin, biglycan and betaglycan proteolysis was mediated by Granzyme B, DCI was included in reactions for 4 h or 24 h (FIG. 4a-c). Higher concentrations of PG substrates and Granzyme B were utilized in this assay for optimal detection of cleavage fragments. DCI effectively inhibited decorin, biglycan and betaglycan cleavage at both time points while the vehicle control (DMSO) had no effect (FIG. 4a-c).

Granzyme B Cleavage Site Identification.

Granzyme B cleavage sites were characterized in biglycan and betaglycan by Edman degradation (FIG. 4b-c). N-terminal sequence results for decorin were unable to be obtained due to low fragment yields, despite multiple trials. In biglycan, the cleavage site was identified at Asp⁹¹Thr-Thr-Leu-Leu-Asp, with a P1 residue of Asp (FIG. 4b). Interestingly, despite sequencing the 6 unique bands for betaglycan, only one unique cleavage site was characterized, Asp⁵⁵⁸Ala-Ser-Leu-Phe-Thr, near the c-terminus of the protein (FIG. 2c). The n-terminal sequence of betaglycan fragments labeled by 1 corresponded to the n-terminus of the protein and the n-terminal sequence of fragments labeled with 2 corresponded to the cleavage site (FIG. 4c). The difference in apparent sizes in the SDS-PAGE gel is most likely due to differences in glycosylation. This heterogeneity in glycosylation is evident in the full length protein as it runs as a smear at the top of the gel (denoted by * in FIG. 4c).

Referring to FIG. 4, it is demonstrated therein that Granzyme B-mediated cleavage of PGs is inhibited by DCI at 4 h and 24 h and Granzyme B cleavage sites contain aspartic acid at the P1 residue. More specifically, Granzyme B was incubated with decorin (a), biglycan (b) and betaglycan (c), +/−DCI and the solvent control DMSO, for 4 h and 24 h. Cleavage sites in biglycan and betaglycan were identified by N-terminal Edman degradation. As utilized therein, the mark * denotes full length protein, arrows indicate cleavage fragments, and cleavage sites are displayed on the right.

Granzyme B-Dependent Cleavage of Biglycan, Decorin and Betaglycan Results in the Release of Active TGF-β1.

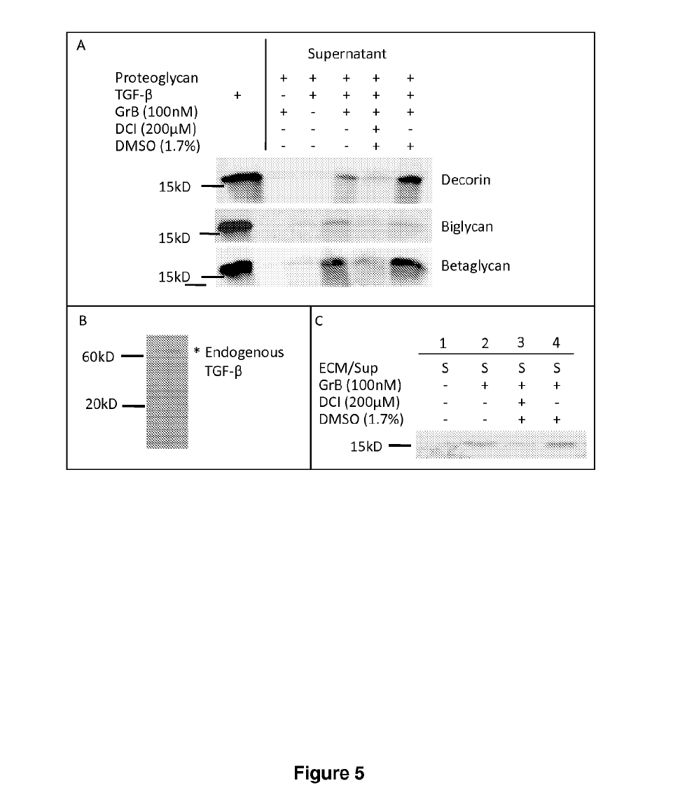

As decorin, biglycan and betaglycan sequester active to TGF-β1, a TGF-β release assay was utilized to determine if Granzyme B-mediated cleavage of these proteins resulted in active TGF-β release (FIG. 5). Following 24 h of incubation, minimal TGF-β had dissociated from the plate in the absence of Granzyme B, suggesting that the PG/TGF-β complexes were stable throughout the incubation time. After 24 h of Granzyme B treatment, TGF-β was released into the supernatants, from all three PG's. This release was inhibited by DCI, suggesting the process was dependent on active Granzyme B. Betaglycan consistently released more TGF-β than decorin and biglycan.

Referring to FIG. 5, Granzyme B cleavage of decorin, biglycan and betaglycan is demonstrated to result in the release of active TGF-β. More specifically, 48 well plates coated with TGF-β1 bound decorin, biglycan and betaglycan were treated with Granzyme B, DCI, and/or the inhibitor solvent control for 24 h. Supernatants (containing released TGF-β) were collected and released TGF-β was detected by Western blotting. This is a representative western blot from 2-3 repeats for each PG.

TGF-β Released by Granzyme B Remains Active and Induces SMAD and Erk Signaling in Smooth Muscle Cells.

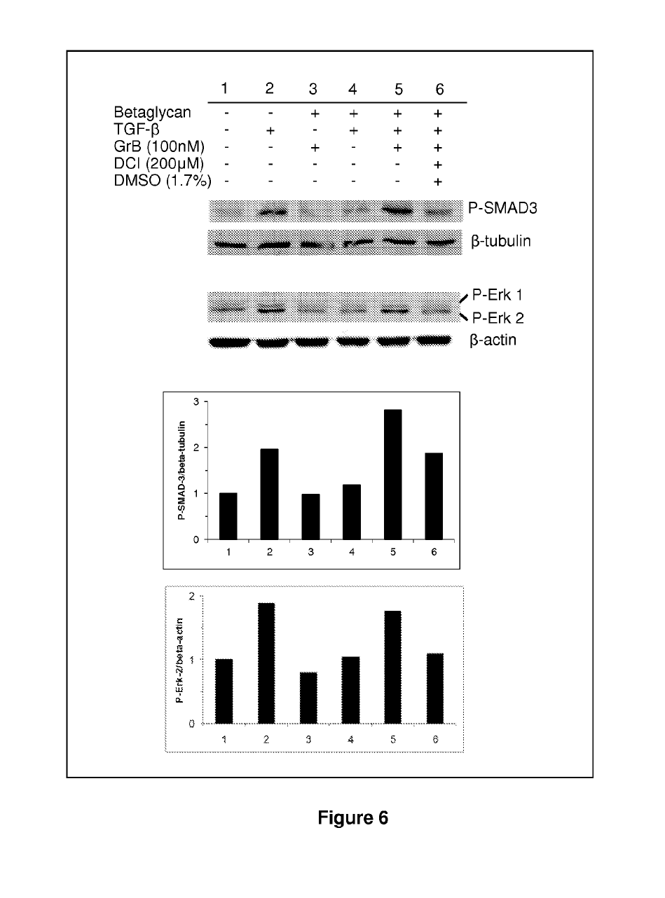

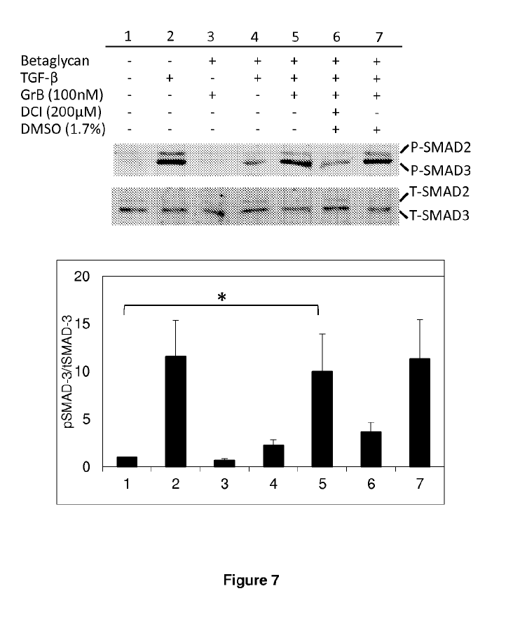

To determine that the TGF-β released by Granzyme B remained active and was not bound to an inhibitory fragment, supernatants from the betaglycan release assay were incubated on human coronary artery smooth muscle cells for 16 h (FIG. 6). TGF-β signaling was examined through the phoshoporylation and activation of SMAD-3 and Erk 1/2. HCASMCs responded well to the 10 ng TGF-β positive control group, with increased SMAD-3 and Erk 1/2 phosphorylation at 16 h. The TGF-β released from betaglycan by Granzyme B induced SMAD and Erk signaling, confirming that the TGF-β released by Granzyme B remained active. As expected, there was limited TGF-β signaling in the absence of Granzyme B or in the presence of DCI. Total Erk and total SMAD levels did not change with TGF-β treatment. Referring to FIG. 6, TGF-β which is released by Granzyme B is active and induces SMAD-3 and Erk-2 phosphorylation in HCASMCs. More specifically, Granzyme B+/−DCI was incubated on betaglycan/TGF-β complexes for 24 h. Supernatants (containing released TGF-β) were added to HCSMC for 16 h and phosphorylated Erk as well as phosphorylated SMAD-3 levels were examined. A similar trend in Granzyme B-dependent phosphorylation was observed in two additional experiments (FIG. 7).

In the foregoing Examples, three novel extracellular substrates of Granzyme B were identified: decorin, biglycan and betaglycan. Furthermore, it was demonstrated that upon cleavage of these PGs by Granzyme B, active TGF-β is released. Reports have indicated that approximately one third of Granzyme B may be released non-specifically during immune cell engagement/degranulation and cytotoxic lymphocytes constitutively release Granzyme B in the absence of target cell engagement (see, for e.g. Prakash et al., 2008). Based, in part, on the results obtained herein, Granzyme B plays an extracellular role in pathogenesis. In this study the Granzyme B cleavage sites for biglycan and betaglycan were identified. In this study, it was demonstrated that Granzyme B cleaved these PG substrates at a P1 residue of Asp (biglycan: D⁹¹, betaglycan: D⁵⁵⁸). Further, in the studies described herein TGF-β1 was released from all three substrates. There was no release evident in the negative control lacking protease or when Granzyme B was co-incubated with the irreversible inhibitor DCI. In addition, the TGF-β released by Granzyme B induced SMAD-3 and Erk-2 phosphorylation, thereby confirming that Granzyme B releases active TGF-β and does not alter TGF-β activity.

Betaglycan cleavage consistently released more TGF-β than biglycan and decorin, which may be due to betaglycan having several binding sites for TGF-β and Granzyme B potentially releasing the cytokine from more than one binding site.

In summary, the current Example demonstrates the identification of three novel factors for Granzyme B, and demonstrates how an accumulation of Granzyme B in the extracellular milieu negatively impacts growth factor sequestration by the ECM.

### Example 4

### A Role for Granzyme B in Matrix Remodelling and Aging of the Skin in Apolipoprotein E Knockout Mice

Abbreviations Used Herein:

apolipoprotein E (apoE); knockout (KO); double knockout (DKO); extracellular matrix (ECM); Granzyme B (Granzyme B); ultraviolet (UV); high fat diet (HFD); second harmonic generation (SHG).

Materials and Methods:

Mice. All animal procedures were performed in accordance with the guidelines for animal experimentation approved by the Animal Care Committee of the University of British Columbia. Male C57BL/6 and apoE-KO mice were purchased from The Jackson Laboratory (Bar Harbor, Me.) and housed at The Genetic Engineered Models (GEM) facility (James Hogg Research Centre, UBC/St. Paul's Hospital, Vancouver, BC). ApoE/Granzyme B double knockout mice were generated on site and also housed at the GEM facility. All mice were fed ad libitum on either a high fat (21.2% fat, TD.88137, Harlan Teklad; Madison, Wis.) or regular chow (equal pans PicoLab Mouse Diet 20: 5058 and PicoLab Rodent Diet 20: 5053, LabDiet; Richmond, Ind.) diet beginning at 6-8 weeks of age for either 0, 5, 15 or 30 weeks. At their respective time points, mice were weighed, and euthanized by carbon dioxide inhalation. Life span was measured using only mice designated for the 30 week time point and mortality the result of required euthanasia due to severe illness in the form of open skin lesions and xanthomatous lesions. The degree of disease severity requiring euthanasia was determined in a blinded manner by an independent animal care technician within the GEM facility. Briefly, animals were considered for euthanasia if they appeared to be in distress or pain that could not be alleviated. Because the animals cannot receive pain medication, mice deemed to be suffering because of open skin lesions or severe xanthomas required euthanasia.

Tissue Collection and Processing.

Following euthanasia, mouse back hair was shaved and dorsal skin was removed from the mid to lower back. Half of the skin sample was fixed in 10% phosphate buffered formalin. Fixed skin sections were processed, embedded in paraffin and cut to 5 μm cross-sections for histology and immunohistochemistry. The other half of the dorsal skin sample was treated with a hair removing cream to completely remove all hair from the surface of the skin. These skin samples were then flash frozen in liquid N₂ and stored at −80° C. until further use for multi-photon microscopy.

Histology and Immunohistochemistry.

Paraffin embedded skin cross-sections were stained with hematoxylin and eosin (H&E) for evaluation of morphology and with picrosirius red to examine collagen content. Luna's elastin was used to examine elastic fibres. Measurement of skin thickness was completed using a 40× objective lens and a calibrated ocular micrometer scale. Measurements were taken across the entire cross-sectional surface of the skin at multiple sites and averaged for each mouse. Collagen was observed in picrosirius red stained sections using 100% polarized light and pictures were taken at a fixed exposure. Granzyme B immunohistochemistry was performed by boiling deparaffinised slides in citrate buffer (pH 6.0) for 15 min. Background staining was blocked by incubating slides with 10% goat serum. The primary antibody used was a rabbit anti-mouse Granzyme B antibody at a 1:100 dilution (Abcam, Cambridge, Mass.) and was incubated at 4° C. overnight. Slides were then incubated with biotinylated goat anti-rabbit secondary antibody at a 1:350 dilution (Vector Laboratories, Burlingame, Calif.) followed by ABC reagent (Vector Laboratories). Staining was visualized with DAB peroxidise substrate (Vector Laboratories). Decorin immunohistochemistry was performed by immersing deparaffinised slides in citrate buffer (pH 6.0) at 80° C. for 10 min. Slides were blocked with 10% rabbit serum and a goat anti-mouse decorin antibody (1 μg/ml) (R&D Systems, Minneapolis, Minn.) was used while slides incubated at 4° C. overnight. Biotinylated rabbit anti-goat secondary antibody was used (1:350) (Vector Laboratories) along with ABC reagent (Vector Laboratories) and DAB substrate (Vector Laboratories) as described above.

Multi-Photon Microscopy.

Frozen skin samples with the hair completely removed were thawed at room temperature and immobilized on a fat surface inside a small dish. Skin samples were washed several times and immersed in phosphate buffed saline. Second harmonic generation (SHG) signals were emitted by the collagen in the skin samples and quantified as a measure of collagen density. Methods used were similar to those described previously (Abraham et al., 2009). Briefly, the laser used was a mode-locked femto-second Ti:Sapphire Tsunami (Spectra-Physics, Mountain View, Calif.) and was focused on the specimen through a 20×/0.5 NA HCX APO L water dipping objective. An excitation wavelength of 880 nm was used and backscattered SHG emissions from the sample were collected through the objective lens. Leica Confocal Software TCS SP2 was used for the image acquisition. Images (8 bit) acquired were frame-averaged 10 times to minimize the random noise. For each sample, about 200-250 Z-section images with a thickness of about 0.63 μm were acquired at decreasing tissue depths for a total thickness measurement of approximately 130-160 μm per sample. These measurements were taken completely within the dermis of each sample as the thinnest dermal layer observed was 250 μm, therefore any decrease in signal is due to a decrease in density rather than a lack of dermal collagen material. Z-section images were compiled and finally the 3D image restoration was performed using Volocity software (Improvisions, Inc., Waltham, Mass.). A noise-removal filter whose kernel size of 3×3 was applied to these 3D images and SHG signals that fell within a set threshold were quantified for the entire 3D image using Velocity software (Improvisions Inc.).

Statistical Analysis.

Survival data were analyzed for significance using the Mantel-Cox test with P<0.05 considered significant. One- or two-way ANOVA with Bonferroni post test was used where appropriate for group comparison analyses with P<0.05 considered significant.

Results:

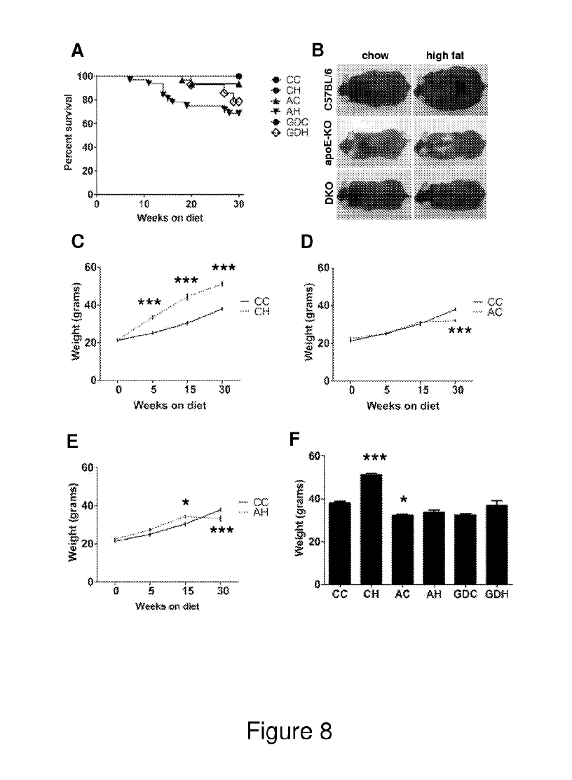

Morbidity and skin pathology. All cases that required euthanasia prior to 30 weeks were attributed to severe open or xanthomatotic-skin lesions. Consistent with previous reports, apoE-KO mice in this study exhibited a marked decline in health compared to wild type controls resulting in increased morbidity and frequency of required euthanasia over a 30 week span (FIG. 8A). While placing wild type mice on a HFD did not alter survival over the 30 week span, the necessity for euthanasia was significantly increased when the apoE-KO mice were fed a HFD with only about 69% surviving to the 30 week time point (FIG. 8A).

As shown in FIG. 8B, apoE-KO mice exhibited signs of frailty, hair loss, hair graying and the formation of subcutaneous lesions or xanthomas on their backs and shoulders at 30 weeks. These phenotypes were more severe and occurred much earlier when apoE-KO mice were fed a HFD (FIG. 8B). Of all apoE-KO mice on a regular chow diet in the 30 week group, 9/31 (29%) demonstrated evidence of xanthoma/skin pathologies with the earliest case at 18 weeks and the majority of the cases (7/9) appearing when examined at 30 weeks. When fed a HFD, 13/32 (41%) apoE-KO mice showed evidence of xanthomas/skin pathology with 10/13 occurring prior to the 30 week time point. These data demonstrate that a HFD accelerates the frequency and onset of these lesions. Interestingly, the appearance of severe xanthomas was delayed in the HFD-fed DKO mice as the first case requiring euthanasia appeared at 19.9 weeks with only 3/14 (21%) total incidence of observed skin pathologies (FIG. 8A). By comparison, at 19.9 weeks, 8 mice from the HFD-fed apoE-KO group (25%) already required euthanasia, with the first occurring as early as 7 weeks (Table 1). DKO mice fed a regular chow diet appeared to develop xanthomas in some cases (2/11 or 18%) but were never severe enough to require euthanasia prior to 30 weeks (FIG. 8A). These results demonstrate that Granzyme B contributes to lesion severity and that reduced Granzyme B delays the onset of these skin pathologies. Table 1 summarizes the incidence and severity of the skin lesions in all groups.

TABLE 1
Summary of xanthoma/skin pathology incidence. CC: C57BL/6 Chow; CH:
C57BL/6 High Fat; AC: apoE-KO Chow; AH: apoE-KO High Fat;
GDC: DKO Chow; GDH: DKO High Fat.

| | CC | CH | AC | AH | GDC | GDH |
|---|---|---|---|---|---|---|
| Total incidence of xanthoma/skin | 0/19 | 0/18 | 9/31 | 13/32 | 0/11 | 3/14 |
| pathology | (0%) | (0%) | (29%) | (41%) | (0%) | (21%) |
| Skin pathologies resulting in | 0/19 | 0/18 | 2/31 | 10/32 | 0/11 | 3/14 |
| premature euthanasia | (0%) | (0%) | (6%) | (31%) | (0%) | (21%) |
| Skin pathology identified at 30 weeks | 0/19 | 0/18 | 7/31 | 3/32 | 2/11 | 0/14 |
| | (0%) | (0%) | (23%) | (9%) | (18%) | (0%) |

Weight Gain.

While a HFD resulted in significant weight gain in C57BL/6 control mice at the 30 week time point; apoE-KO mice on a HFD showed no significant increase in weight compared to the chow fed apoE-KO mice and weighed significantly less than the HFD-fed C57BL/6 mice (FIG. 8F) at 30 weeks. When weight gain was examined at the 0, 5, 15 and 30 week time points, C57BL/6 mice on a HFD showed a significant increase in weight as early as 5 weeks on the diet compared to the chow fed controls which remained higher throughout the course of the study (FIG. 8C). ApoE deficiency alone resulted in no difference in weight gain compared to the chow-fed controls until 30 weeks when the chow-fed apoE-KO group stopped gaining weight (FIG. 8D) and adopted a frail phenotype (FIG. 8B). When apoE-KO mice were fed a HFD, they appeared to gain weight at a similar rate to the control group and possibly to a greater extent at 15 weeks. However, by 30 weeks they appeared to have actually lost weight (FIG. 8E) and often displayed frail and diseased skin (FIG. 8B). To examine the role of Granzyme B in this process, DKO mice on either a regular chow or a HFD were maintained until 30 weeks. Both groups of DKO mice showed no significant difference in weight at 30 weeks compared to either the C57BL/6 chow-fed control group or the apoE-KO groups (FIG. 8F).

Referring to FIG. 8, C57BL/6 chow (CC), C57BL/6 high fat (CH), apoE-KO chow (AC), apoE-KO high fat (AH), DKO chow (GDC) and DKO high fat (GDH). (A) All C57BL/6 wild type mice survived to the 30 week time point on either a high fat (n=18) or regular chow (n=19) diet while 94% of chow-fed apoE-KO mice (n=31) were kept alive for 30 weeks. A HFD significantly (P<0.01) reduced survival in apoE-KO mice (n=32) compared to the control CC group with only 69% remaining healthy enough to survive for the 30 week span. 100% of chow-fed DKO mice (n=11) survived to the 30 week time point while morbidity in the DKO mice fed a HFD (n=14) appeared to be delayed compared to the AH group. (B) Representative images of mice at the 30 week time point. (C-E) Weight gain over 0, 5, 15 and 30 weeks for the CH, AC and AH groups compared to CC. (F) Average weights of the all groups of mice at the 30 week time point (Error bars represent the mean±SEM). (D-F). *P<0.05, ***P<0.001.

More specifically with respect to FIG. 8B, the photographs therein depict as follows: C57BL/6 Chow—control mouse, typical healthy size and weight. Normal looking black hair. C57BL/6 High Fat—appears obese compared to the control mouse, hair and skin look otherwise normal. ApoE-KO Chow—this mouse appears frail compared to the control mouse, shows evidence of hair graying and some areas of hair thinning/loss. ApoE-KO High Fat—this mouse also appears more frail compared to the control mouse and fails to gain weight from the high fat diet as the C57BL/6 mouse does. This mouse also displays evidence of hair graying, hair loss and inflammatory skin lesions (xanthomas) that appear on their backs. DKO Chow—These mice appeared to be relatively normal in terms of weight gain compared to the control mice and reduced incidence and severity of the hair loss, graying and skin lesion formation compared to the apoE-KO Chow group. DKO High Fat—this group also generally appeared healthier than the apoE-KO High Fat group with reduced incidence and severity of hair loss, graying and skin lesions.

Skin Histopathology.

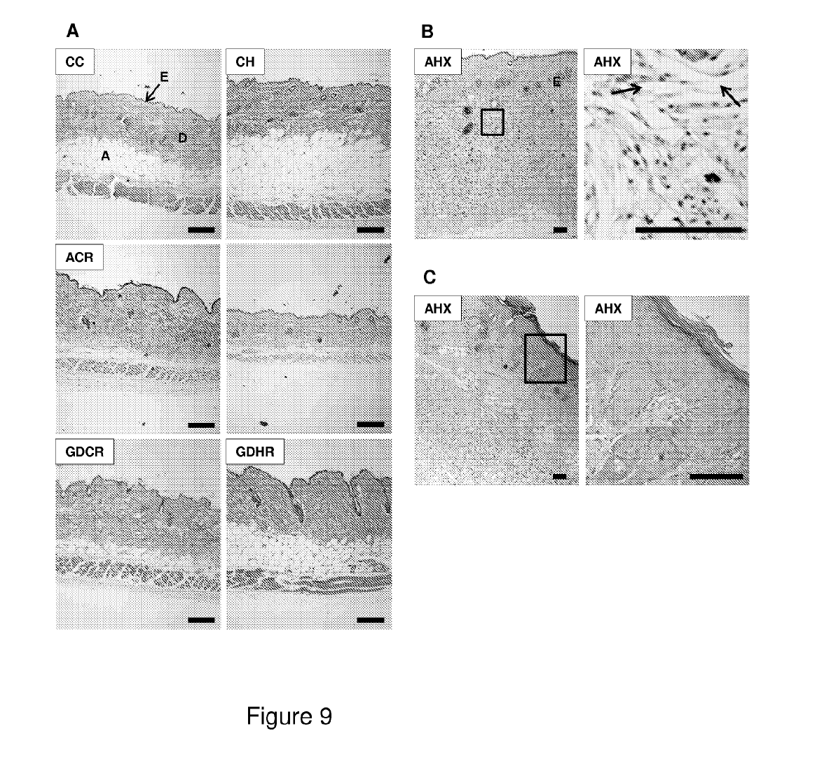

As shown in FIG. 9, the skin of apoE-KO mice is heterogeneous; exhibiting normal “regular” looking skin (FIG. 9A) and other areas featuring xanthomatous lesions (FIG. 9B). These lesions often develop on the backs of the mice and occur with increased severity and frequency with age and when fed a HFD. None of the C57BL/6 wild type mice exhibited xanthomatosis at any time point over the week span regardless of diet. Histological examination of the xanthomatous lesions in apoE-KO mice revealed skin thickening including that of the epidermis, considerable immune infiltration, loss of normal adipose tissue, ECM alterations and the presence of cholesterol crystals (FIG. 9B). Although xanthoma development was common in apoE-KO mice, not all mice displayed this phenotype and in some instances mice had skin that appeared relatively normal (FIG. 9A).

Referring to FIG. 9, ApoE-KO mouse skin is heterogeneous with certain areas of the skin appearing “regular” while other areas contain xanthomatous lesions (H&E stain). (A) “Regular” looking skin from C57BL/6 chow (CC), C57BL/6 high fat (CH), apoE-KO chow (ACR), apoE-KO high fat (AHR), DKO chow (GDCR) and DKO high fat (GDHR) at the 30 week time point. (B) Xanthoma from a HFD-fed apoE-KO mouse (AHX) that survived to the 30 week time point demonstrating cholesterol crystals (arrows) and (C) from a HFD-fed apoE-KO mouse that required euthanasia at 14.9 weeks due to the severity of the lesion. E: epidermis; D: dermis; A: adipose tissue. (A scale bar=200 μm, B and C scale bar=100 μm).

Skin Thickness.

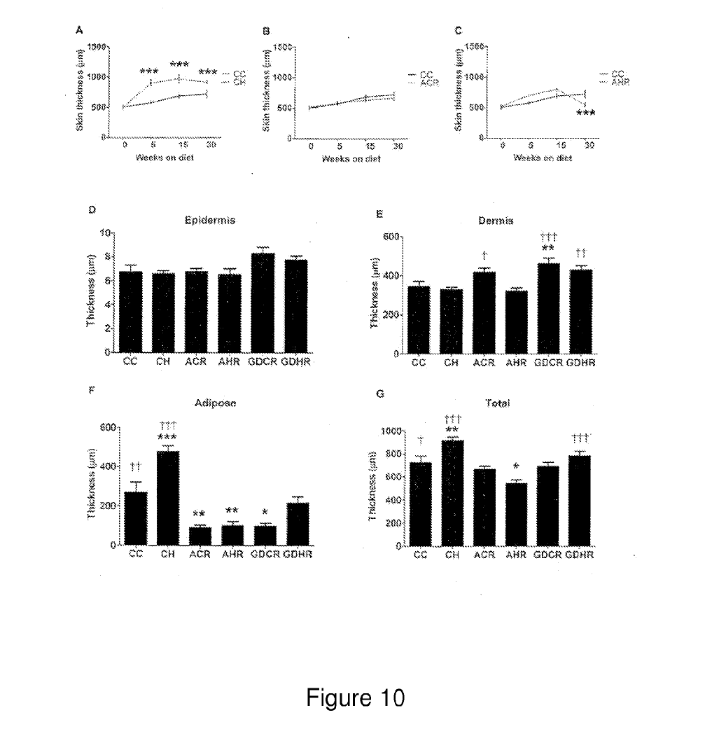

Skin thinning and atrophy is a characteristic feature that occurs with age both in humans and mice (see, for e.g. Bhattacharyya and Thomas, 2004). To determine whether apoE-KO mice exhibit this trait, we analyzed formalin fixed skin sections from the mid to lower back of the chow or HFD-fed C57BL/6 and apoE-KO mice using H&E staining at 0, 5, 15 and 30 weeks and measured total skin thickness including the epidermis, dermis, adipose and skeletal muscle layers (FIG. 10A). Due to the heterogeneous nature of the skin in apoE-KO mice and the fact that xanthoma development has a dramatic effect on skin thickness (FIG. 9), skin samples from apoE-KO mice were separated into two groups depending on the histological presence of xanthoma: “regular” skin and xanthoma skin. When fed a HFD, wild type C57BL/6 mice had significantly thicker skin than the control group at 5, 15 and 30 week time points (FIG. 10A). ApoE deficiency alone resulted in no significant difference over time compared to controls as shown in FIG. 10B. Interestingly, skin from the HFD-fed apoE-KO group, while significantly thinner than the control group at 30 weeks, appeared to be slightly thicker than the control group at 15 weeks when thickness was measured over time (FIG. 10C). This demonstrates that these changes are not simply a result of developmental differences but rather a change that occurs at an accelerated rate over time compared to the wild type controls. To determine whether Granzyme B deficiency protects against skin thinning and frailty, skin from DKO mice on a regular chow or HFD was examined at 30 weeks and compared to the other groups. As mentioned above, the HFD caused a significant increase in skin thickness in the C57BL/6 group while a significant decrease in thickness in the apoE-KO group (FIG. 10G). HFD-fed DKO mice displayed a significant increase in total skin thickness compared to the HFD-fed apoE-KO group at 30 weeks (FIG. 10G), demonstrating that Granzyme B contributes to age-related skin thinning and frailty in apoE-KO mice.

Closer analysis of the individual layers of the skin revealed that changes in total skin thickness in the “regular” skin samples were due primarily to changes in the dermal and/or adipose tissue layers. While no significant differences were observed in epidermal thickness at the 30 week time point for any of the groups (FIG. 10D), dermal thickness was significantly thicker in the chow-fed DKO group compared to the chow-fed control group (FIG. 10E). Interestingly, when fed a HFD, the DKO group had a significantly thicker dermis than the HFD-fed apoE-KO group (FIG. 10E). Changes in the thickness of the adipose tissue layer varied the most with the HFD-fed C57BL/6 group demonstrating a significant increase and both apoE-KO groups and the chow-fed DKO group having significantly thinner adipose tissue layer than the chow-fed controls (FIG. 10F). HFD-fed DKO mice showed no significant difference in adipose tissue thickness compared to either the chow-fed controls or the apoE-KO groups (FIG. 10F). These results demonstrate that apoE deficiency results in a decrease in total skin thickness and that this is due in pan to a decrease in adipose tissue while Granzyme B deficiency protects against skin thinning due in part to an increase in dermal thickness.

Referring to FIG. 10, (A-C) skin thickness of C57BL/6 chow (CC), C57BL/6 high fat (CH), apoE-KO chow (ACR) and apoE-KO high fat (AHR) was measured at 0, 5, 15 and 30 weeks using non-diseased “regular” skin sections. Individual skin layers were measured for CC, CH, ACR, AHR, DKO chow (GDCR) and DKO high fat (GDHR) at 30 weeks including the (D) epidermis, (E) dermis, (F) adipose and (G) total skin thickness including skeletal muscle. Error bars represent the mean±SEM. *P<0.05 vs CC; **P<0.01 vs CC; ***P<0.0 vs CC; tP<0.05 vs AHR; ††P<0.01 vs AHR; †††P<0.001 vs AHR.

Collagen and Elastin Abnormalities in the Skin of apoE-KO Mice.

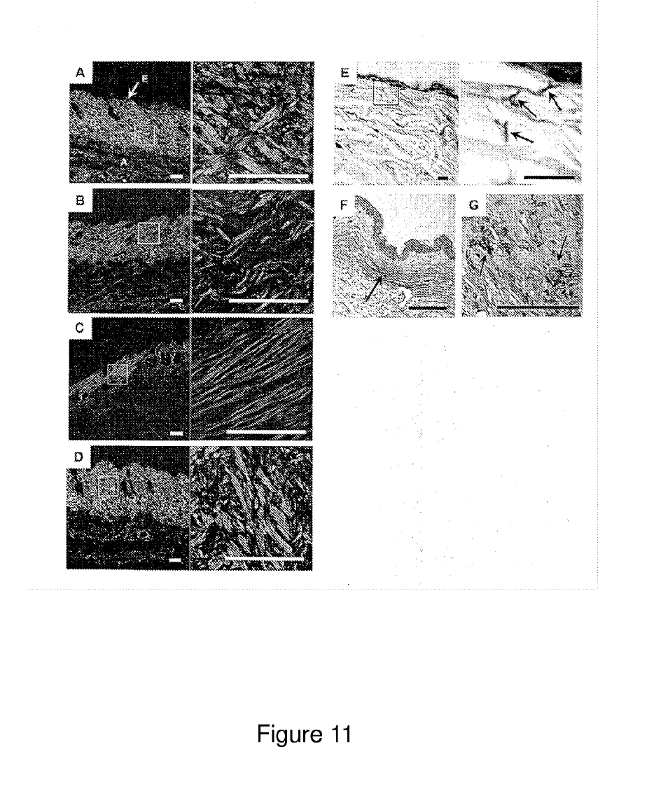

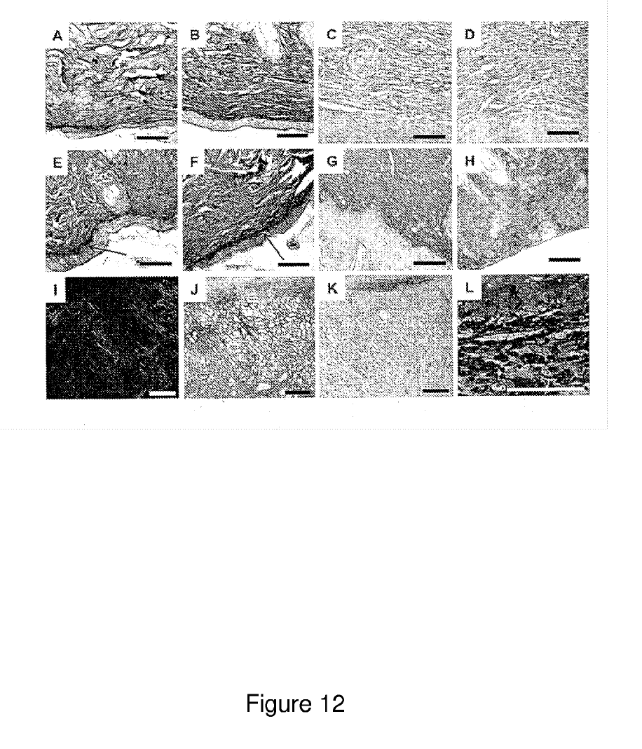

To investigate the collagen changes occurring in the diseased, xanthoma skin lesions of apoE-KO mice, skin sections were stained with picrosirius red and visualized using polarized light. As shown in FIGS. 11C and 12K, skin lesions display clear alterations in collagen organization and structure compared to regular skin from control mice (FIG. 11A). Collagen fibres were often arranged in a more parallel orientation with thinner collagen bundles in the diseased skin (FIG. 11C), which explains the increased stiffness and skin frailty that was observed in these lesions. Some areas of the dermis displayed a near complete loss/degradation of normal collagen and evidence of damage to the dermal-epidermal barrier (FIG. 12I).

To examine elastin content in the diseased skin, Luna's elastin stain was used. Wild type control mice demonstrated diffuse elastin distribution with thin elastic fibres and minimal large elastin bundles (FIG. 11E). In the diseased skin, we observed increased elastin deposition in the papillary dermis as well as abnormal elastin bundle deposits in the dermis (FIGS. 11F and G).

Referring to FIG. 11, skin sections from chow-fed C57BL/6 mice display thick dense collagen fibres while apoE-KO mice on a HFD frequently display areas of altered collagen morphology with reduced density compared to controls. (A) Images of skin collagen from a C57BL/6 mouse on a chow diet for 30 weeks (E: epidermis; D: dermis; A: adipose tissue). (B) Skin collagen from a “regular” skin sample from an apoE-KO mouse on a HFD for 30 weeks. (C) HFD-fed apoE-KO mouse skin collagen from a diseased area containing xanthoma. (D) Example of skin collagen from “regular” skin of a HFD-fed DKO mouse at 30 weeks. (E) Elastin from C57BL/6 mouse on a chow diet for 30 weeks (Elastin stains dark purple—arrows). (F and G) Examples of abnormal elastin deposits (arrows) from 30 week HFD-fed apoE-KO mice with diseased skin. Picrosirius red stain viewed under polarized light (A-D) and Luna's elastin stain (E-G). (A-D and F-G scale bars=100 μm, E scale bars=10 μm).

In FIG. 11, collagen is monitored. In FIG. 11A, collagen is densely packed in this slide from a normal (non-knock-out mouse). FIG. 11B is a slide from an apoE-ko mouse. The collagen appears to be packed less densely. In FIG. 11C, this slide shows diseased skin from an apoE-ko mouse. The collagen appears to be linear and is less elastic. In FIG. 11D, this slide shows collagen in a Granzyme B−/− ApoE-ko mouse. The collagen appears to be packed more densely compared with the single knockout apoE-ko mouse tissue. In FIGS. 11E and 11F, elastin is monitored. FIG. 11E is data from a normal mouse. The right panel shows elastin fibers (see arrows).

Decorin Remodelling and Granzyme B Expression in the Skin apoE-KO Mice.

Collagen disorganization was readily observed in the diseased skin of apoE-KO mice (FIG. 12I). While minimal differences in some areas of the skin were observed (FIG. 12B), staining the diseased skin sections with anti-decorin antibody revealed a distinct to loss of decorin in other areas of the diseased skin in apoE-KO mice (FIGS. 12C and D). Areas of decorin degradation corresponded with areas of collagen loss and remodelling (FIGS. 12I and J). DKO mice exhibited increased decorin content in the non-diseased skin sections particularly near the dermal epidermal junction (FIGS. 12E and F). Additionally, areas of decorin loss were not observed in the diseased skin sections from DKO mice to the same extent as apoE-KO mice (FIGS. 12G and H). When xanthoma skin sections from apoE-KO mice were stained with anti-Granzyme B antibody, Granzyme B was observed in the lesion in specific areas and was often localized to the papillary dermis near the dermal-epidermal junction (FIG. 12K). Interestingly, these areas of localized Granzyme B expression in the lesions directly corresponded with areas of collagen/decorin loss and remodelling as evidenced by staining serial sections (FIGS. 12I-K). These results demonstrate that Granzyme B plays a role in collagen remodelling in the skin through the cleavage of decorin.

Referring to FIG. 12, decorin staining in the skin from (A) chow-fed C57BL/6 (B-D) HFD-fed apoE-KO, (E-F) HFD fed DKO “regular” skin showing darker decorin staining at the papillary dermis near the dermal epidermal junction (arrows) and (G-H) DKO diseased skin. Serial sections of HFD-fed apoE-KO skin stained for (1) collagen, (J) decorin and, (K and L) Granzyme B. (A-H and L scale bars=50 μm, I-K scale bars=200 μm)

In FIG. 12A decorin staining is shown in a normal mouse. The staining is more intense towards the epidermal-dermal junction. FIG. 12B shows decorin staining in an apoE-ko mouse. The staining is more diffuse. FIGS. 12C and 12D show nearly absent decorin staining in diseased portions of skin from apoE-KO mouse tissue. FIGS. 12E and 12F show regular skin from Granzyme B−/−apoE−/− mouse tissue. There is intense decorin staining. FIGS. 12G and 12H show diseased skin from Granzyme B−/−apoE−/− mouse tissue. In FIG. 12H decorin is shown in the epidermis. FIGS. 12I, 12J, and 12K are serial sections monitoring collagen, decorin, and Granzyme B respectively, all from apoE-ko mouse tissue. FIG. 12L is a zoom-in photo from FIG. 12K. There is increased Granzyme B staining in FIG. 12K.

Collagen Density and Organization.

To determine if apoE-KO mice exhibit differences in collagen content in the regular, non-xanthoma skin, picrosirius red staining was used on formalin fixed skin sections and analyzed for changes in collagen content and structure. Dermal collagen from the chow-fed control group exhibited typical red/orange staining of thick, dense collagen fibres at the 30 week time point (FIG. 11A). In contrast, HFD-fed apoE-KO mice often displayed dermal collagen that was loosely packed, and less structured that the control group (FIG. 11B). This was not readily observed in the other groups including the HFD-fed DKO group (FIG. 11D) suggesting a HFD combined with apoE deficiency affects collagen structure and density in apoE-KO mouse skin, and that Granzyme B deficiency prevents the loss of thick fibre formation and collagen density.

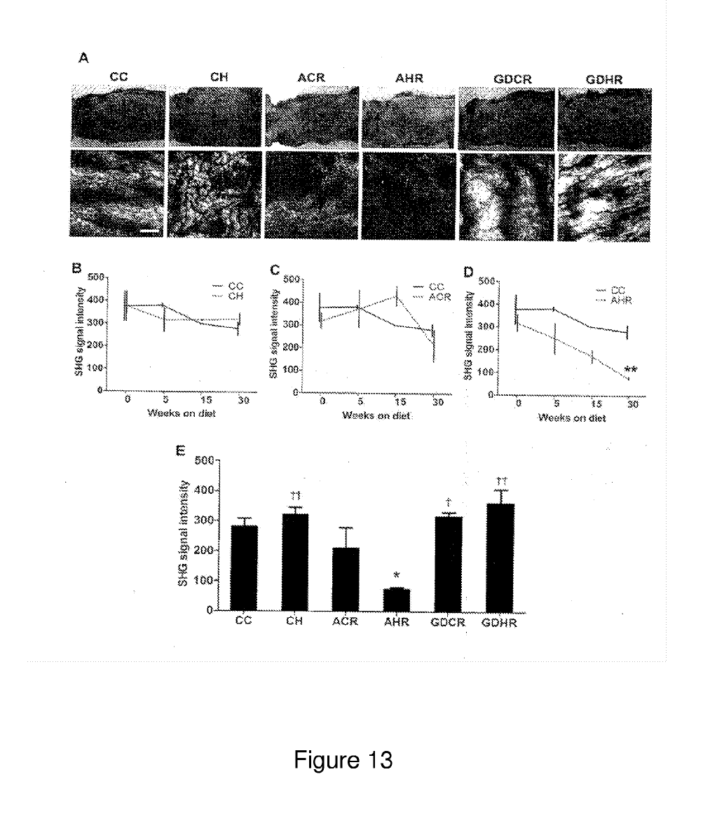

Although analysis of fixed, thin sliced sections can provide useful information regarding collagen content and structure, important three dimensional and organizational properties may be missed or altered during processing. We took advantage of the bifringent properties of collagen to visualize collagen structure and organization in unfixed, unstained thick skin samples in three dimensional space using multi-photon microscopy. Highly ordered fibril-forming collagens (Type I, II, III, etc.) produce second harmonic generation (SHG) signals without the need for any exogenous label (see, for e.g., Zipfel et al., 2003). These SHG signals correlate with the density and organization of the collagen matrix rather than total collagen content. Representative flattened three dimensional SHG images originating from the collagen matrix (grey) are shown in FIG. 13A for all groups at the 30 week time point. Only non-diseased skin was used for these experiments to ensure any ECM changes observed were not the result of xanthoma formation but rather the result of a more intrinsic aging process. When collagen density was monitored over time, the 0 week time point appeared similar for the C57BL/6 and apoE-KO groups (FIG. 13B-D). The chow-fed control group appeared to show a slight decrease in collagen density over time while the apoE-KO mice fed a HFD exhibited a reduced SHG signal as a function of age beginning at 5 weeks and at a greater rate than the wild type controls demonstrating an increased rate of collagen modification and disorganization resulting in a significant loss of collagen density by 30 weeks (FIG. 13D). To confirm that the skin used in these experiments was regular, non-xanthoma skin, skin samples were fixed in 10% buffered formalin following these experiments and examined histologically using H&E staining. Upon histological examination the HFD-fed apoE-KO skin chosen for these experiments did not show evidence of xanthomatosis, confirming the observed changes in collagen density are an intrinsic property, rather than brought on by xanthoma formation (data not shown). These results demonstrate that age-related ECM changes are occurring in the skin of HFD-fed apoE-KO mice even in skin sections without xanthoma formation at an increased rate compared to wild type mice leading to premature skin aging.

Referring to FIG. 13, (A) representative 3D merged plane images of fresh ex-vivo unfixed, unstained mouse “regular” skin tissues obtained from chow fed C57BL/6 (CC), apoE-KO mice (ACR) and DKO mice (GDCR) and HFD-fcd C57BL/6 (CH), apoE-KO mice (AHR) and DKO mice (GDHR) at 30 weeks. Gray colors represent the collagen matrix. (B-D) collagen density as a function of time expressed as the intensity of the SHG signal for the CC, CH, ACR and AHR groups (0, 5, 15 and 30 weeks on their respective diets). (C) Collagen density at the 30 week time point for all six groups. Knocking out Granzyme B restores the SHG signal intensity that is lost in the HFD-fed apoE-KO mice. Error bars represent the mean±SEM. *P<0.05 vs CC; **P<0.01 vs CC; †P<0.05 vs AHR; ††P<0.01 vs AHR. Scale bars=80 μm.

To further examine the role of Granzyme B in the observed loss of collagen density, DKO mice skin was also examined using SHG after being fed a chow or HFD for 30 weeks as this was the time point where the most extreme differences were observed. As mentioned, at the 30 week time point only the HFD-feed apoE-KO mice exhibited significantly decreased collagen density in the skin compared to the chow-fed control group as shown by the decreased SHG signal (FIG. 13E) suggesting apoE deficiency combined with a HFD result in a loss of skin collagen density. Both groups of DKO mice on either diet demonstrated a significant increase in skin collagen density when compared to the HFD-fed apoE-KO group, deonstrating that Granzyme B plays a role in the intrinsic aging process in the skin by facilitating the age-dependant disorganization of dermal collagen (FIG. 13E).

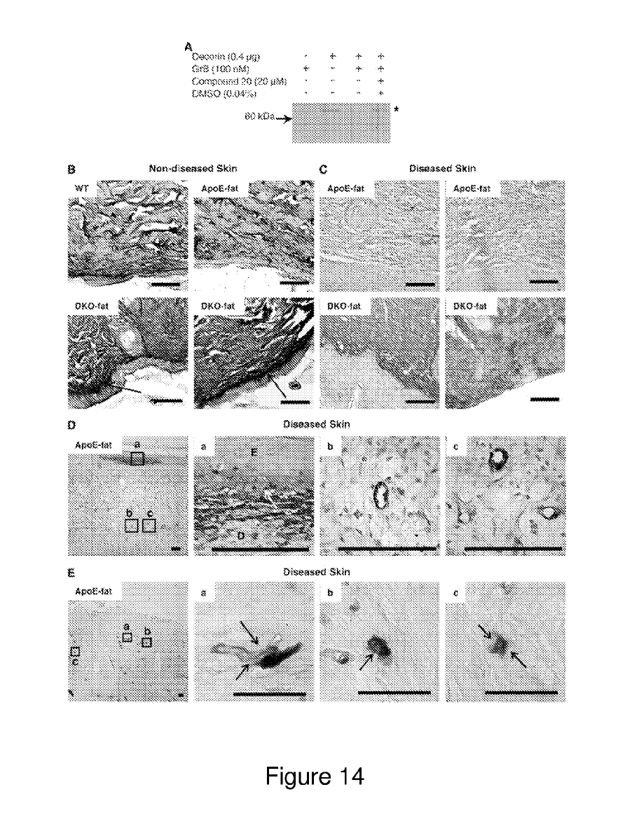

Granzyme B cleaves decorin and is present in areas of decorin degradation. Referring to FIG. 14, the addition of Granzyme B results in degradation and loss of full-length glycosylated decorin by 24 h. This is prevented when the potent Granzyme B inhibitor, compound 20, is included. (FIG. 14A; Asterisk=full length protein). Decorin immunostaining (dark gray) in the non-diseased skin from wild type chow (WT), apoE-KO high fat (ApoE-fat) and DKO high fat (DKO-fat) groups. Arrows point to increased decorin near the dermal-epidermal junction (scale bars=50 μm) (FIG. 14B). Decorin immunostaining (dark gray) in diseased skin from ApoE-fat and DKO-fat groups (scale bars=50 μm) (FIG. 14C). Granzyme B immunostaining (dark gray) in diseased skin from an ApoE-fat mouse. E denotes “epidermis” and D denotes “dermis”. Arrow points to the area of the dermal-epidermal junction (scale bars=100 μm) (FIG. 14D). Diseased skin section from ApoE-fat mice dual stained for mast cells and Granzyme B. Arrows point to Granzyme B (light gray) inside mast cells (dark gray) (scale bars=25 μm) (FIG. 14E).

Discussion of Results in the Foregoing Example:

In the present study, it was demonstrated that a HFD has a considerable effect on skin aging in apoE-KO mice. Not only does it affect the frequency of inflammatory skin disease as the mice age, but also results in a frail, thinned skin state along with significant age-related alterations in the structural organization of the ECM. We also demonstrate that the serine protease, Granzyme B, plays an important role in aging and disease of the skin through remodelling of key ECM proteins and proteoglycans. When apoE-KO mice were fed a HFD for 30 weeks, they demonstrated frailty and increased morbidity compared to the wild type controls (FIG. 8). This was also observed histologically in the form of increased skin lesions and skin thinning along with a loss of subcutaneous adipose tissue (FIGS. 9 and 10). Although xanthoma development occurred regardless of diet in apoE-KO mice, a HFD was required to observe certain intrinsic aging phenotypes such as skin thinning and loss of collagen density (FIGS. 10 and 13). Similar to the onset and severity of skin lesions in apoE-KO mice, a HFD accelerates these aging characteristics and chow-fed apoE-KO mice also display these phenotypes upon examination at a later time point beyond 30 weeks.

In this study, apoE-KO mice fed a regular chow or HFD showed a decrease in adipose layer thickness at 30 weeks while overall skin thickness in the HFD-fed apoE-KO mouse skin decreased in from the 15 to 30 week time point (FIG. 10). This demonstrates that Granzyme B deficiency helps to rescue this skin thinning phenotype in part by preserving dermal thickness (FIG. 10). Analysis of collagen and elastin in the xanthoma skin samples demonstrated considerable remodelling of collagen along with abnormal elastin deposits resembling solar elastosis often seen in photoaged skin. The presence of age-related changes in the “regular” skin of apoE-KO mice, together with the thickened, remodelled, pro-inflammatory state of the xanthoma skin demonstrate that apoE-KO mice demonstrate features of both intrinsic/chronological skin aging and extrinsic aging similar to photoaging with both resulting in ECM changes that mimic these forms of skin aging in humans.

In this study evidence is provided that the serine protease Granzyme B is expressed in areas of collagen and decorin degradation and remodelling in the skin of apoE-KO mice (FIG. 12). Our results also demonstrate that Granzyme B contributes to the frail/thin skin and lack of dense collagen observed in aged apoE-KO mice.

Further, the lichenoid expression of Granzyme B observed in the diseased skin samples presents a novel mechanism of lesion formation and ECM degradation. Lichenoid inflammation is a characteristic feature of several inflammatory skin diseases. The presence of Granzyme B in this area also shows that Granzyme B is disrupting ECM close to or at the dermal epidermal junction. Indeed, DKO mice demonstrated an apparent increase in decorin staining in the skin near the dermal epidermal junction (sec. for e.g., FIGS. 12E and F).

In addition to the diseased skin, apoE-KO mice fed a HFD, but not a regular chow diet, demonstrated a significant loss in collagen density in the dermis as shown by SHG and multi-photon microscopy over a 30 week span in “regular” skin samples (FIG. 13). These experiments were done on fresh thick tissue sections providing a unique look at the collagen content of the skin in 3D prior to any fixation or processing methods. Subsequent fixation and histological analysis of these skin tissues confirmed that the decrease in collagen density in HFD-fed apoE-KO mice occurred in “regular” non-xanthoma skin and that the decreased SHG signal was not simply due to the presence of xanthoma (data not shown). Staining fixed skin sections with picrosirius red also demonstrated a more diffuse collagen pattern in the skin of apoE-KO mice compared to controls (FIG. 11B). Interestingly, we show using DKO mice, that this decrease in collagen density and organization signal may be rescued by blocking Granzyme B activity (see, for e.g., FIGS. 11 and 13).

In summary, the findings demonstrate that apoE-deficiency results in an increased pro-inflammatory state in the skin, contributing to ECM remodelling and other age-related changes seen and that a HFD exacerbates these changes through a Granzyme B-mediated mechanism. These findings also demonstrate a novel role for Granzyme B in the skin involving the cleavage of decorin and the remodelling of dermal collagen, a process that has major implications in ECM structure, skin fragility in aging and disease, and wound repair.

### Example 5

### Inhibition of Granzyme B (Granzyme B) Using a Specific Small Molecule Inhibitor (Willoughby 20) Inhibits Betaglycan Cleavage

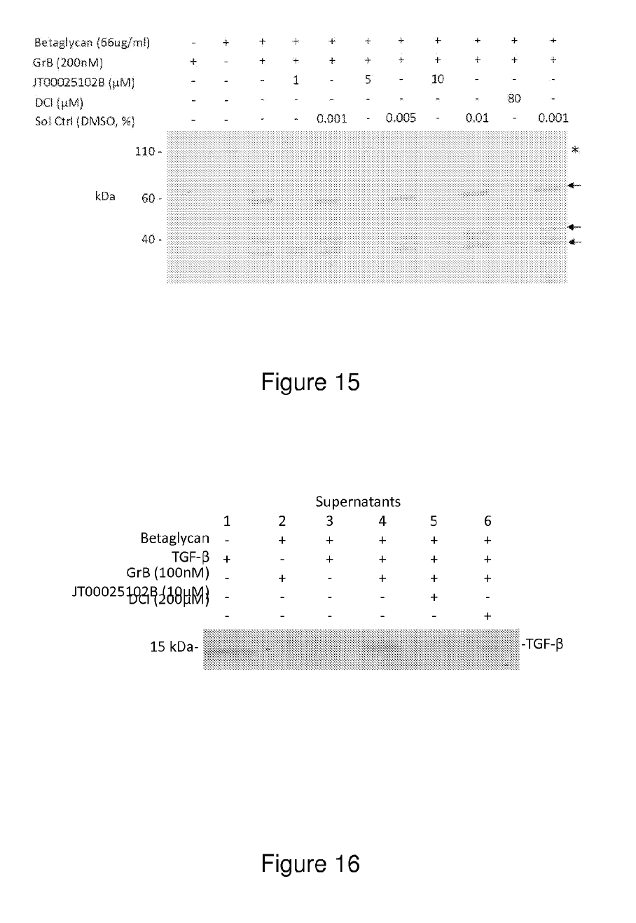

In this Example, inhibition of Granzyme B (Granzyme B) is demonstrated using a specific small molecule inhibitor (Willoughby 20) inhibits betaglycan cleavage. As shown in FIG. 15, incubations were performed at room temperature for 24 hours in a total reaction volume of 30 μl. Samples were run on a 10% gel, imaged with Ponceau stain and scanned. As shown in FIG. 15, the asterisk depicts a full length protein; the arrow depicts cleavage fragments.

### Example 6

### Inhibition of Granzyme B (Granzyme B) Using a Specific Small Molecule Inhibitor (Willoughby 20) Inhibits the Release of Proteoglycan-Sequestered TGF-β

In this Example, 20 ug/ml betaglycan was coated onto 48 well plates and incubated with 10 ng of TGF-β. Excess TGF-β was washed off the plate and betaglycan/TGF-β complexes were incubated with Granzyme B+/− inhibitors for 24 h at RT. Supernatants (containing released TGF-β) were collected and Western blotted for TGF-β. There is little non-specific dissociation of TGF-β into supernatants in the absence of Granzyme B (2; see FIG. 16). TGF-β is released by Granzyme B after 24 h of incubation (4; see FIG. 16). Release is inhibited by Willoughby 20 (specific Granzyme B inhibitor) (5; see FIG. 16) and partially inhibited by DCI (non-specific serine protease inhibitor) (6; see FIG. 16).

### Example 7

### Inhibition of Granzyme II (Granzyme B) Using a Specific Small Molecule Inhibitor (Willoughby 20) Inhibits Decorin Cleavage

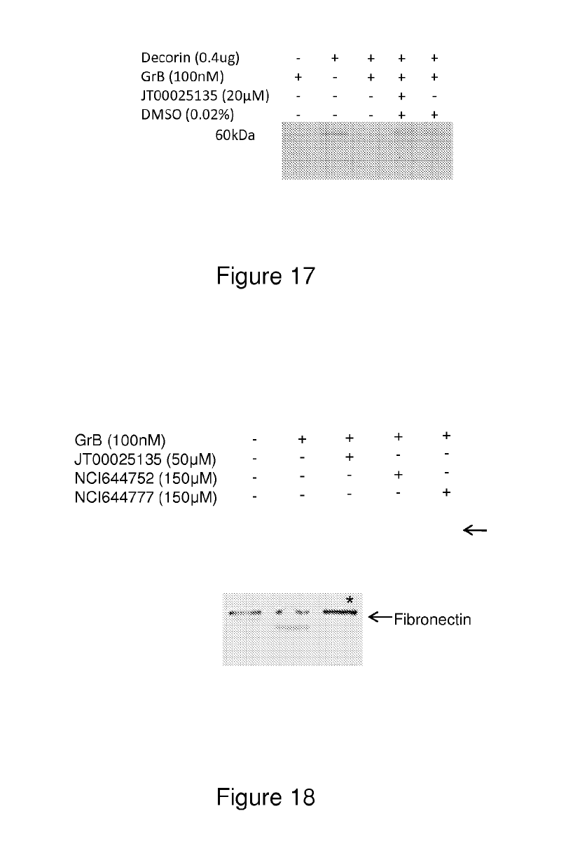

In this Example, incubations were performed at RT for 24 h in a total reaction volume of 30 ul. Samples were run on a 10% gel and imaged by Coomassic Blue stain. With reference to FIG. 17, the asterisk=full length protein.

TABLE A

| | |
|---|---|
| Target | Human Granzyme B |
| Identifier | Azepino[3,2,1-hi]indole-2-carboxamide, 5-[[(2S,3S)-2-[(2- |
| | benzo[b]thien-3-ylacetyl)amino]-3-methyl-1-oxopentyl]amino]- |
| | 1,2,4,5,6,7-hexahydro-4-oxo-N-(1H-1,2,3-triazol-5-ylmethyl)-, |
| | (2S,5S)-; (compound 20 from Willoughby et al. (2002) Bioorganic & |
| | Medicinal Chemistry Letters 12:2197-2200); JT25102B; |
| | JT00025135; Willoughby20 |
| IC₅₀ [inhibition] | ~3.1 μM [exhibited high inhibition] |
| Structure | 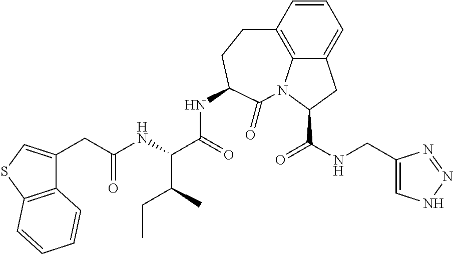 [SMILES: CC[C@H](C)[C@H](NC(=O)Cc1csc2ccccc12)C(=O)N[C@H]1CCc2cccc3c2N(C1=O)[C@H](C(=O)NCc1c[nH]nn1)C3] |

### Example 8

### Identification of Small Molecule Inhibitors of Granzyme B (Granzyme B)

Small molecule libraries (ZINC—Irwin J J and Shoichet B K, 2005. J. Chem Inf Model 4591:177-182; NCI—Voigt, J. H. et al. J. Chem. Inf Comput. Sci. 2001, 41, 702-712) were screened in silico for candidate Granzyme B inhibitory compounds. Several candidate small molecule inhibitors were identified and subjected to an in vitro Granzyme B inhibition assay. More specifically, a continuous colormetric assay for Granzyme B activity was carried out with the substrate Ac-IEPD-pNA. Briefly, 8 ug/ml Granzyme B, 20 μM substrate, and increasing concentrations of the inhibitor of interest was incubated in a final reaction volume of 50 ul. The reaction buffer consisted of 50 mM HEPES pH 7.5, 10% sucrose, 0.1% CHAPS and 5 mM DTT and reactions were carried out at 37° C. pNA release and Granzyme B inhibition was monitored at 405 nm on a Tecan Safire microplate reader. Granzyme B was used in the assay at a concentration of 4 μg/ml (0.145 μM), estimated to be about 80,000 fold higher than what would be observed in a subject; our findings have indicated that pathological levels of Gr B are above 50 pg/ml, to about 150 pg/m. The results are shown in Table B.

TABLE B
Summary of Granzyme B Inhibitor Data.

| | |
|---|---|
| Target | Human Granzyme B |
| Identifier | ZINC05723764; NCI 644752 |
| IC₅₀ [inhibition] | ~25 μM [exhibited high inhibition] |
| Structure | 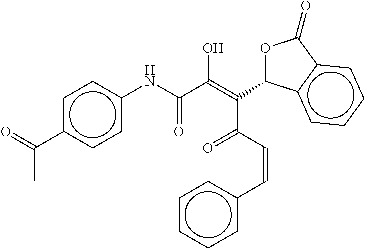 |
| Target | Human Granzyme B |
| Identifier | ZINC05723787; NCI 644777 |
| IC₅₀ [inhibition] | ~40 μM [exhibited high inhibition] |
| Structure | 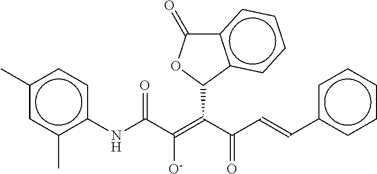 |
| Target | Human Granzyme B |
| Identifier | ZINC05316154; NCI 641248 |
| IC₅₀ [inhibition] | ~195 μM [exhibited low inhibition] |
| Structure | 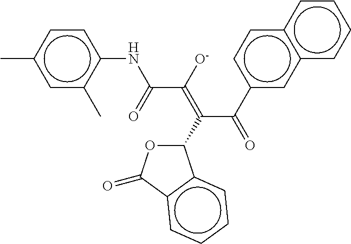 |
| Target | Human Granzyme B |
| Identifier | ZINC05723499; NCI 641235 |
| IC₅₀ [inhibition] | ~224 μM [exhibited low inhibition] |
| Structure | 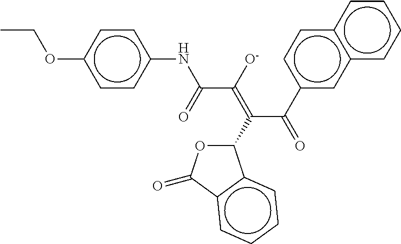 |
| Target | Human Granzyme B |
| Identifier | ZINC05723646; NCI 642017 |
| IC₅₀ [inhibition] | ~250 μM [exhibited low inhibition] |
| Structure | 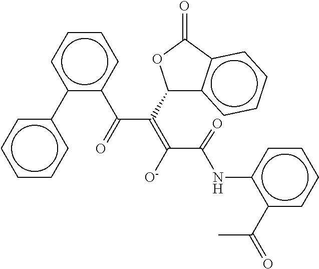 |
| Target | Human Granzyme B |
| Identifier | ZINC05398428; NCI 641230 |
| IC₅₀ [inhibition] | ~255 μM [exhibited low inhibition] |
| Structure | 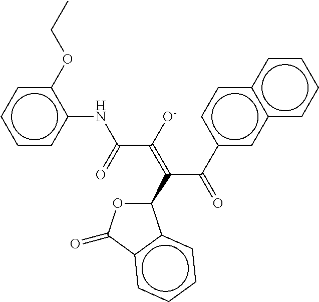 |
| Target | Human Granzyme B |
| Identifier | ZINC05723503; NCI 641236 |
| IC₅₀ [inhibition] | ~270 μM [exhibited low inhibition] |
| Structure | 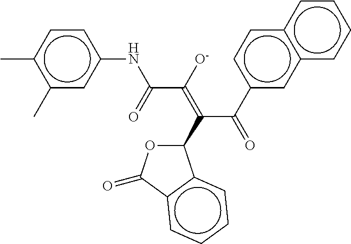 |
| Target | Human Granzyme B |
| Identifier | ZINC05723446; NCI 640985 |
| IC₅₀ [inhibition] | ~270 μM [exhibited low inhibition] |
| Structure | 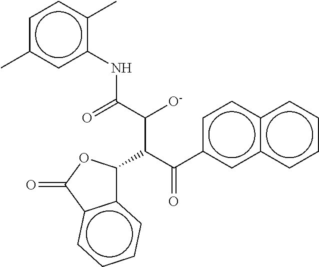 |
| Target | Human Granzyme B |
| Identifier | ZINC05317216; NCI 618792 |
| IC₅₀ [inhibition] | ~50 μM [exhibited high inhibition] |
| Structure | 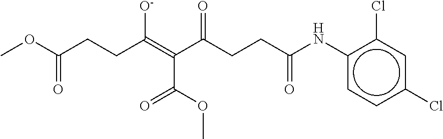 |
| Target | Human Granzyme B |
| Identifier | ZINC05315460; NCI 630295 |
| IC₅₀ [inhibition] | ~100 μM [exhibited high inhibition] |
| Structure | 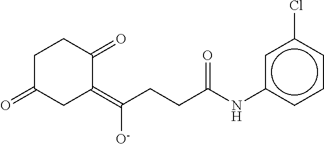 |
| Target | Human Granzyme B |
| Identifier | ZINC05316859; NCI 618802 |
| IC₅₀ [inhibition] | ~250 μM [exhibited low inhibition] |
| Structure | 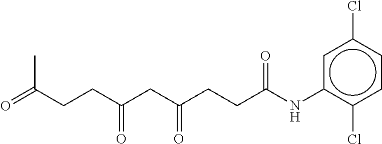 |
| Target | Human Granzyme B |
| Identifier | ZINC05605947; NCI 623744 |
| IC₅₀ [inhibition] | ~320 μM [exhibited low inhibition] |
| Structure | 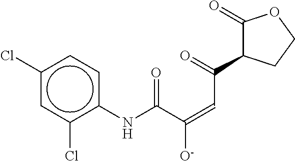 |

As detailed herein and as detailed in Tables A and B herein, candidate inhibitors, and IC₅₀ concentration obtained from the inhibition assay are set out below. Compounds NCI 644752, NCI 644777, ZINC05317216, NCI 630295 demonstrated an IC₅₀ of about 100 μM or less (“High inhibition”); compounds NCI 641248, NCI 641235, NCI 642017, NCI 641230, NCI 641236, NCI 640985, NCI 618802, NCI 623744 demonstrated an IC₅₀ of about 320 μM or less (“Low inhibition”).

### Example 9

### Small Molecule Inhibitors Inhibit Granzyme B Cleavage of Decorin

Details of this Example are shown representatively in FIG. 18. More specifically, FIG. 18 demonstrates that inhibition of Granzyme B (Granzyme B) using small molecule inhibitors inhibits ECM cleavage. As detailed therein, the asterisk marks the full length protein. The arrow demonstrates cleavage fragments and the star denotes the full length protein.

More specifically, as it relates to FIG. 18, to synthesize endogenous ECM, human coronary artery smooth muscle cells were seeded in 6 well plates and incubated at confluency for 7 days in serum starvation media (SMGM+0.2% FBS). Cells were removed from the plates by incubation with 0.25M Ammonium Hydroxide for 20 min. This treatment removes cells while leaving the surrounding extracellular matrix intact. Subsequently, 100 nM Granzyme B and the inhibitor of interest were incubated on the endogenous ECM for 24 h. Supernatants from the plates (containing cleavage fragments) were run on an SDS-PAGE gel and Western blotted for fibronectin or decorin. DMSO solvent control was not included due to redundancy. Asterisk=full length protein; Arrow=cleavage fragments, star denotes full length.

### Example 10

### Inhibition of Granzyme B (Granzyme B) Using a Small Molecule Inhibitor Inhibits ECM Cleavage

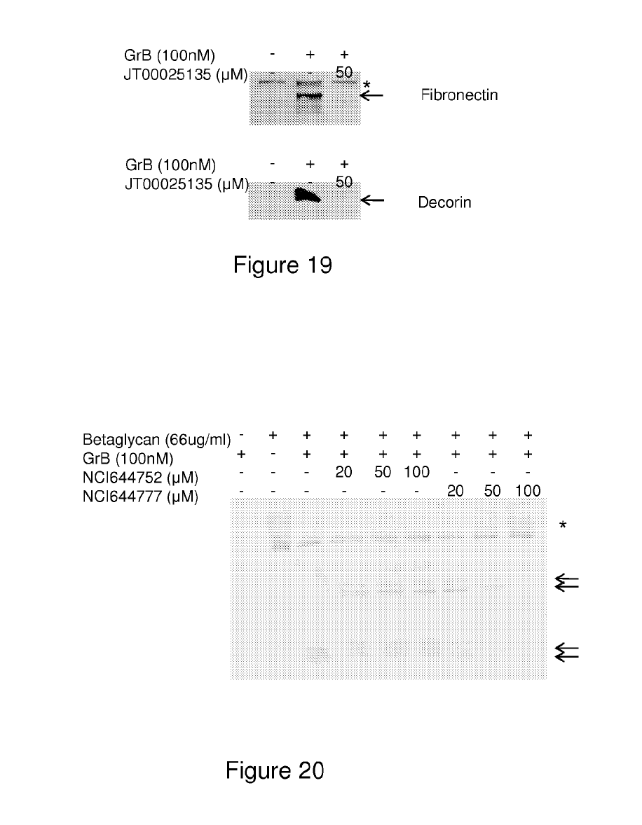

As shown in FIG. 19 herein, to synthesize endogenous ECM, human coronary artery smooth muscle cells were seeded in 6 well plates and incubated at confluency for 7 days in serum starvation media (SMGM+0.2% FBS). Cells were removed from the plates by incubation with 0.25M Ammonium Hydroxide for 20 min. This treatment removes cells while leaving the surrounding extracellular matrix intact. Subsequently, 100 nM Granzyme B and the inhibitor of interest were incubated on the endogenous ECM for 24 h. Supernatants from the plates (containing cleavage fragments) were run on to an SDS-PAGE gel and Western blotted for fibronectin or decorin. DMSO solvent control was not included due to redundancy. Asterisk=full length protein; Arrow=cleavage fragments, star denotes full length.

### Example 11

### Inhibition of Granzyme B (Granzyme B) Using NCI 644777 Inhibits Betaglycan Cleavage

As shown in FIG. 20 herein, incubations were performed at RT for 4 h in a total reaction volume of 30 μl. Samples were run on a 10% gel, imaged with Ponceau stain and scanned. Compound NCI 644752 does not inhibit Granzyme B cleavage of betaglycan at 100 μM (but was previously shown to be effective at 150 μM against decorin cleavage). Compound NCI 644777 partially inhibits Granzyme B at 50 μM and completely at 100 μM. DMSO vehicle control was not included due to redundancy. Asterisk=full length protein; Arrow=cleavage fragments. Obstruction in Granzyme B/Betaglycan lane affecting first 2 cleavage fragments.

### Example 12

### Inhibition of Granzyme B (Granzyme B) Using Willoughby 20 Inhibits Fibronectin Cleavage

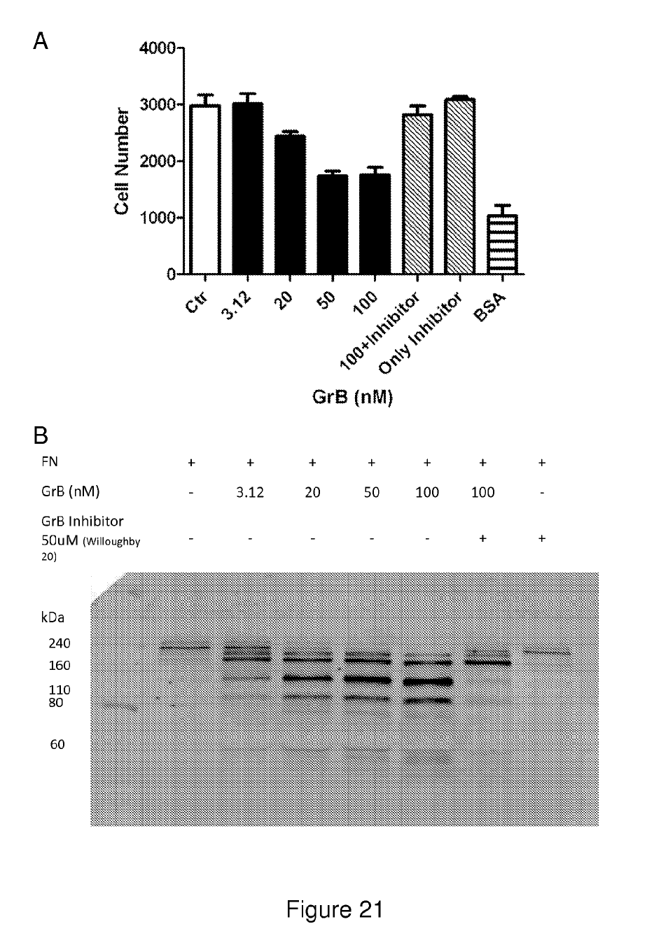

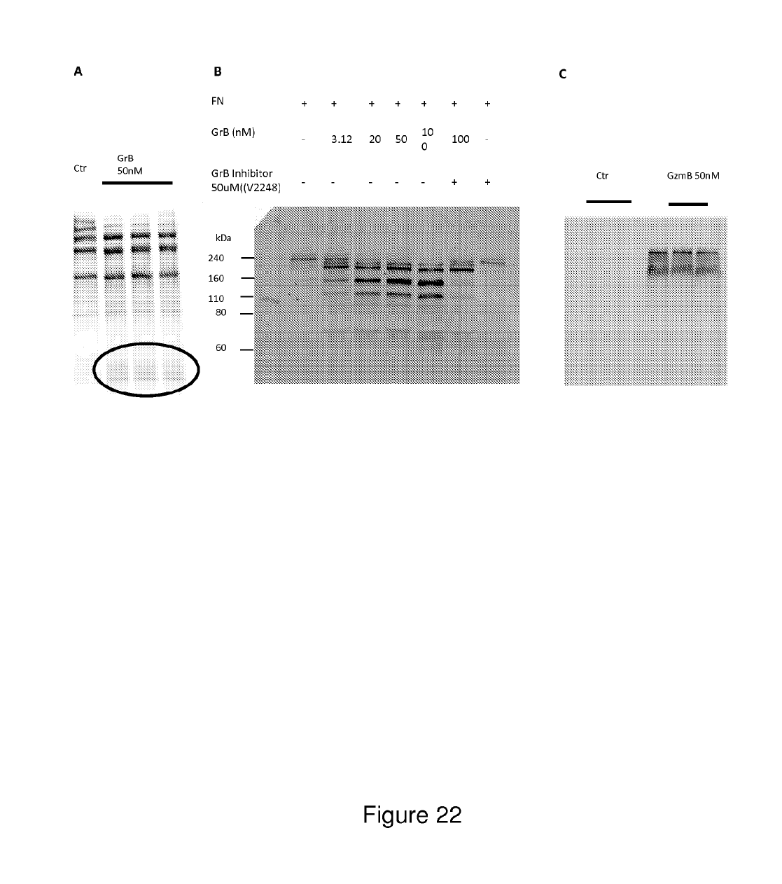

As shown in FIGS. 21A and B herein, incubations as described herein were performed with fibronectin (FN) and Granzyme B, both in the absence of inhibitor Willoughby 20 and in the presence of inhibitor Willoughby 20. Compound Willoughby 20 inhibits Granzyme B cleavage of FN at 3.12 nM. Furthermore, FIG. 21A shows the results of HMVEC addition (Human Microvascular Endothelial Cells) and subsequent cell count and shows that Granzyme B cleavage of fibronectin (FN) reduces EC adhesion to FN dose dependently. FIG. 21B shows Granzyme B specifically and dose dependently cleaves fibronectin resulting in the release of fibronectin fragments.

### Example 13

### Inhibition of Granzyme B Prevents Decorin Degradation in Chronic Wounds In Vivo

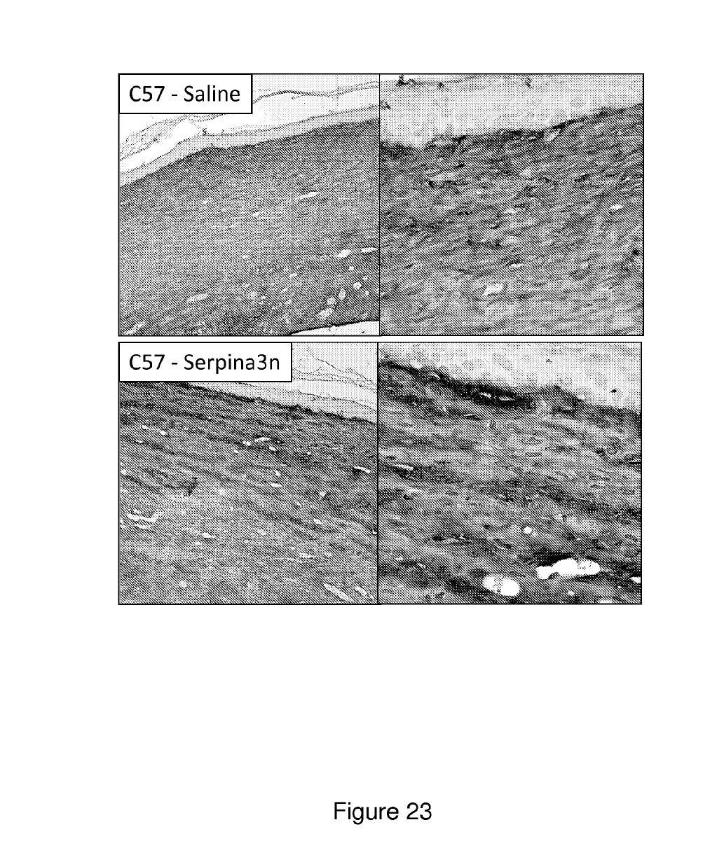

As depicted in FIG. 23, decorin distribution is impacted by serpina3n in closed wound tissue. Seven week old wild type C57BL/6 mice were given a 1 cm diameter full thickness wound on their backs. Mice were then given either saline (upper panels) or the Granzyme B inhibitor, serpina3n (lower panels), by applying 60 μl of the appropriate solution directly onto the wound immediately following the wounding procedure. Wounds were allowed to heal for 16 days, at which point mice were sacrificed and the closed wound tissue harvested. Immunohistochemistry for decorin revealed differences in the pattern of decorin distribution in the newly formed dermis between the two groups. Saline-treated mice demonstrated gradual increase in decorin close to the dermal-epidermal junction. Serpina3n-treated mice demonstrated intense decorin staining near the dermal-epidermal junction but also demonstrated intense decorin staining deeper into the dermis, demonstrating that Granzyme B inhibition prevents excessive decorin degradation in acute or chronic wounds. Increased decorin throughout the newly formed dermis thus provides more organized collagen, less scarring and increased tensile strength.

While specific embodiments of the invention have been described and illustrated, such embodiments should be considered illustrative of the invention only and not as limiting the invention as construed in accordance with the accompanying claims. Other features and advantages of the invention will be apparent from the following description of the drawings and the invention, and from the claims.

INFORMAL SEQUENCE LISTINGS
(SEQ ID NO: 1)
Asp⁹¹Thr-Thr-Leu-Leu-Asp <biglycan cleavage sequence>
(SEQ ID NO: 2)
Asp⁵⁵⁸Ala-Ser-Leu-Phe-Thr <betaglycan cleavage sequence>
(SEQ ID NO: 3)
Asp³¹Glu-Ala-Ser-Gly <decorin cleavage sequence>
(SEQ ID NO: 4)
Asp⁶⁹Leu-Gly-Asp-lys <decorin cleavage sequence>
(SEQ ID NO: 5)
Asp⁸²Thr-Thr-Leu-Leu-Asp <decorin cleavage sequence>
(SEQ ID NO: 6)
Asp²⁶¹Asn-Gly-Ser-Leu-Ala <decorin cleavage sequence>
Human Granzyme B amino acid sequence:
(SEQ ID NO: 7)
MQPILLLLAFLLLPRADAGEIIGGHEAKPHSRPYMAYLMIWDQKSLKRCGGFLIRDDFVLTAAHCWGSSINVTLGAHNI
KEQEPTQQFIPVKRPIPHPAYNPKNFSNDIMLLQLERKAKRTRAVQPLRLPSNKAQVKPGQTCSVAGWGQTAPLGKHSH
TLQEVKMTVQEDRKCESDLRHYYDSTIELCVGDPEIKKTSFKGDSGGPLVCNKVAQGIVSYGRNNGMPPRACTKVSSFV
HWIKKTMKRY
Human Granzyme 8 nucleotide sequence:
(SEQ ID NO: 8)
CCAAGAGCTAAAAGAGAGCAAGGAGGAAACAACAGCAGCTCCAACCAGGGCAGCCTTCCTGAGAAGATGCAACCAATCC
TGCTTCTGCTGGCCTTCCTCCTGCTGCCCAGGGCAGATGCAGGGGAGATCATCGGGGGACATGAGGCCAAGCCCCACTC
CCGCCCCTACATGGCTTATCTTATGATCTGGGATCAGAAGTCTCTGAAGAGGTGCGGTGGCTTCCTGATACGAGACGAC
TTCGTGCTGACAGCTGCTCACTGTTGGGGAAGCTCCATAAATGTCACCTTGGGGGCCCACAATATCAAAGAACAGGAGC
CGACCCAGCAGTTTATCCCTGTGAAAAGACCCATCCCCCATCCAGCCTATAATCCTAAGAACTTCTCCAACGACATCAT
GCTACTGCAGCTGGAGAGAAAGGCCAAGCGGACCAGAGCTGTGCAGCCCCTCAGGCTACCTAGCAACAAGGCCCAGGTG
AAGCCAGGGCAGACATGCAGTGTGGCCGGCTGGGGGCAGACGGCCCCCCTGGGAAAACACTCACACACACTACAAGAGG
TGAAGATGACAGTGCAGGAAGATCGAAAGTGCGAATCTGACTTACGCCATTATTACGACAGTACCATTGAGTTGTGCGT
GGGGGACCCAGAGATTAAAAAGACTTCCTTTAAGGGGGACTCTGGAGGCCCTCTTGTGTGTAACAAGGTGGCCCAGGGC
ATTGTCTCCTATGGACGAAACAATGGCATGCCTCCACGAGCCTGCACCAAAGTCTCAAGCTTTGTACACTGGATAAAGA
AAACCATGAAACGCTACTAACTACAGGAAGCAAACTAAGCCCCCGCTGTAATGAAACACCTTCTCTGGAGCCAAGTCCA
GATTTACACTGGGAGAGGTGCCAGCAACTGAATAAATACCTCTTAGCTGAGTGGAAAAAAAAAAAAAAAAAA
Huamn Serpin B9
(SEQ ID NO: 9)
METLSNASGTFAIRLLKILCQDNPSHNVFCSPVSISSALAMVLLGAKGNTATQMAQALSLNTEEDIHRAFQSLLTEVNK
AGTQYLLRTANRLFGEKTCQFLSTFKESCLQFYHAELKELSFIRAAEESRKHINTWVSKKTEGKIEELLPGSSIDAETR
LVLVNAIYFKGKWNEPFDETYTREMPFKINQEEQRPVQMMYQEATFKLAHVGEVRAQLLELPYARKELSLLVLLPDDGV
ELSTVEKSLTFEKLTAWTKPDCMKSTEVEVLLPKFKLQEDYDMESVLRHLGIVDAFQQGKADLSAMSAERDLCLSKFVH
KSFVEVNEEGTEAAAASSCFVVAECCMESGPRFCADHPFLFFIRHNRANSILFCGRFSSP
Huamn Serpin B9
(SEQ ID NO: 10)
GCGGGAGTCCGCGGCGAGCGCAGCAGCAGGGCCGGGTCCTGCGCCTCGGGGGTCGGCGTCCAGGCTCGGA
GCGCGGCACGGAGACGGCGGCAGCGCTGGACTAGGTGGCAGGCCCTGCATCATGGAAACTCTTTCTAATG
CAAGTGGTACTTTTGCCATACGCCTTTTAAAGATACTGTGTCAAGATAACCCTTCGCACAACGTGTTCTG
TTCTCCTGTGAGCATCTCCTCTGCCCTGGCCATGGTTCTCCTAGGGGCAAAGGGAAACACCGCAACCCAG
ATGGCCCAGGCACTGTCTTTAAACACAGAGGAAGACATTCATCGGGCTTTCCAGTCGCTTCTCACTGAAG
TGAACAAGGCTGGCACACAGTACCTGCTGAGAACGGCCAACAGGCTCTTTGGAGAGAAAACTTGTCAGTT
CCTCTCAACGTTTAAGGAATCCTGTCTTCAATTCTACCATGCTGAGCTGAAGGAGCTTTCCTTTATCAGA
GCTGCAGAAGAGTCCAGGAAACACATCAACACCTGGGTCTCAAAAAAGACCGAAGGTAAAATTGAAGAGT
TGTTGCCGGGTAGCTCAATTGATGCAGAAACCAGGCTGGTTCTTGTCAATGCCATCTACTTCAAAGGAAA
GTGGAATGAACCGTTTGACGAAACATACACAAGGGAAATGCCCTTTAAAATAAACCAGGAGGAGCAAAGG
CCAGTGCAGATGATGTATCAGGAGGCCACGTTTAAGCTCGCCCACGTGGGCGAGGTGCGCGCGCAGCTGC
TGGAGCTGCCCTACGCCAGGAAGGAGCTGAGCCTGCTGGTGCTGCTGCCTGACGACGGCGTGGAGCTCAG
CACGGTGGAAAAAAGTCTCACTTTTGAGAAACTCACAGCCTGGACCAAGCCAGACTGTATGAAGAGTACT
GAGGTTGAAGTTCTCCTTCCAAAATTTAAACTACAAGAGGATTATGACATGGAATCTGTGCTTCGGCATT
TGGGAATTGTTGATGCCTTCCAACAGGGCAAGGCTGACTTGTCGGCAATGTCAGCGGAGAGAGACCTGTG
TCTGTCCAAGTTCGTGCACAAGAGTTTTGTGGAGGTGAATGAAGAAGGCACCGAGGCAGCGGCAGCGTCG
AGCTGCTTTGTAGTTGCAGAGTGCTGCATGGAATCTGGCCCCAGGTTCTGTGCTGACCACCCTTTCCTTT
TCTTCATCAGGCACAACAGAGCCAACAGCATTCTGTTCTGTGGCAGGTTCTCATCGCCATAAAGGGTGCA
CTTACCGTGCACTCGGCCATTTCCCTCTTCCTGTGTCCCCAGATCCCCACTACAGCTCCAAGAGGATGGG
CCTAGAAAGCCAAGTGCAAAGATGAGGGCAGATTCTTTACCTGTCTGCCCTCATGATTTGCCAGCATGAA
TTCATGATGCTCCACACTCGCTTATGCTACTTAATCAGAATCTTGAGAAAATAGACCATAATGATTCCCT
GTTGTATTAAAATTGCAGTCCAAATCCCATAGGATGGCAAGCAAAGTTCTTCTAGAATTCCACATGCAAT
TCACTCTGGCGACCCTGTGCTTTCCTGACACTGCGAATACATTCCTTAACCCGCTGCCTCAGTGGTAATA
AATGGTGCTAGATATTGCTACTATTTTATAGATTTCCTGGTGCTTAGCCTTATAAAAAAGGTTGTAAAAT
GTACATTTATATTTTATCTTTTTTTTTTTTTTTTTTCTGAGACGCAGTCTGGCTCTCTGTCGCCCAGGCT
GGAGTGCAGTGGCTCGATCTCGGCTCACTGCAAGCTCCGCCTCCCGGGTTCACGCCATTCTCCTGCCTCA
GCCTCCCGAGTAGCTGGGACTACAGGCGCCCGCCACCACGCCCGGCTAATTTTTTGTATTTTTAGTAGAG
ACGGGGTTTCACCGTGTTAGCCAGGATGGTGTCGATCTCCTGACCTCGTGATCCACCCGCCTCGGCCTCC
CAAAGTGCTGGGATTACAGGCTTGAGCCACCGCGCCCGGCTATATTTTATCTTTTATCTTTTTCTTTGAC
ATTTACCAATCACCAAGCATGCACCAAACACTGCTTTAGGCACTGGGGACACAAAGGGGACAGAGCCATC
CTCCTTTGACACCTGGTCTTCAGTTCTGTGCCCAACGTATATAGTTTTGACAATGACCAGGTTGGACTGT
TTAATGTCTTTCAACTTACCACGTAATCCTCTTGTAGGGATCACATCTTTCTTTATGATATTGTATTTCT
CTACCTCTAACAGTAAAAATTCCATTCAACCCTTAAAGCTCACTTCAAATTCTTCTTTGAGAAGTTTTTC
CTTTCTCCGCAACCAGATGTACATATTTGAACTCTCTTTGTACTTGGAGGGCACTTCTTTCGTGGTAGTT
CTTTTATTTTTATTAATCTCTGTATCCTTAGATAGTCCTCCAACAACCAAAGGTTGGGACTCTGTCTTAC
ATATCTGGGTGCCCCTCATAGTGCAGTAATAAGTAAGTTGATTATATACGAGCTATGTAACTTATATTTT
TTAATGGTTGGATATCACTGAGTTTTTTTTTTTAAGAATTTTTTTATTGAGGTAAACTTCACATAACATA
AAATTAACTATTTTAAAGTGAGAAGTTCAGTGCCACTTAGTATTGTTAACAATGTTGCATAACCACCACC
TTTATTTAAAGTTCCAAAAAAAATGTTCTCCTCTAAAAGGAAACCCCATCCCATTAAGCAGATACTCTCC
ATTCCTTCCTTCCTCCAGCCCCCAGCAACCACCAATCTGCTTTCTGTCTCTATGGATTTATCTATTCTTG
CTATTTTATATAAATTGAATTGTATGAGACCTTTTGTGTCTGGCTTCTTTCACTTAGTACAAGTTTTTGA
GATTTATTTACATAGTAGCATGTATCAACACTTCATTTTTATGGCCAAATAAAATTGTATTATGTGTTTA
TAGCACAATTTATTTATCCACTCATTCATTGATGGACTTTGGGTTGTTTCTGACTTTTGGCTATTGGGAA
TAGTGCTGCTATGAATGTTTGTGTACCTGTATTTGTTTGAATGCCTATTTTGCATTCTCTTGGGTATATA
TCTAGGAGTGGAACTGCTGGGTCATATGTTAATTCTATGTTTAGCTTTTTGAGGAACAGACAAACTGTTT
TCCACAGCAGTTGAACCATTCCACATTCCCACCAGCAATGTATGAGAATTCCAATTTCTGTCCACTTCCT
CACCAACACTTATTATTTTCCTTTTCCTTTTTTTAAAAAAAATAAGTTATGGCCATCTTAGTGGGTGTGA
AGTGGTATCTCATTGTGTTTTTTATTTGCATTTCCTATGTAATGAGCTAGAAACTAAAGTACAAACTAGA
TGGGACATCCAGTCCCTTTGATAGATAATGCTGAGTAAAAAATGAGATGAAAGACATTTGTTTGTTTTTA
GAACACGAGTGACAGTTTGTTAAAAAGCTTTAGAGGAGGAATGAAAACAAAGTGAAGTACACTTAGAAAA
GGGCCAAGTGGACATCTTGGATGTCAAGTGCCTAGTTCAGTATCTTTTTTTTTTTTTTTTTTTTTTTTGA
GACAGTGCCTCACTCTGTCACCCAGGCTGGAGTGTAGTGGCATGATCTGGGCTCACTGCAACCTCCTCCT
CCTGGATTCAAGCAATTCTCTTGCTTCAGCCTCCCAAGTAGCTGAGACTACAAGCACCCACCATCACACC
CAGCTAATTTTGTATTTTTCAGTAGAGACGGGGTTTCGCCACATTGGCCGTGTTGGTCTTGAACTCCTGG
CCTCAAGCGATCCGCCTACCTCAGCCTCCCAAAGTGCTAGGATTACAGGCATAAGCCACTGAGCCCAGCC
CTAGTTCAGTATCTTTTATGTAAATTACAAACATCTGCAACATTATGTATCATATGCAGATACTTATTGC
ATTTCTTTTATTAGTGGTGAAAGTGTTCTATGCATTTATTGGCTCTTGAATTTCCTCATCTATGAATTGT
CATTCATACACCTACTTTTCTGCTTCGTTTTTACATATGTCTTTGCCTATTAAAGATATTATCCCTCTGT
TTTATATTTTCTCTCATTCTTGTATTGCCTTTTAAATTTTGTTATGATGTTTCATTAATAAACAGTGTTT
TGTTTTCCTCTATAAAAAAAAAAAAAAAA
Fragment from Huamn Serpin B9
(SEQ. ID NO: 11)
GTEAAASSCFVAECCMESG
Human SerpinA3
(SEQ ID NO: 12)
MERMLPLLALGLLAAGFCPAVLCHPNSPLDEENLTQENQDRGTHVDLGLASANVDFAFSLYKQLVLKAPDKNVIFSPLS
ISTALAFLSLGAHNTTLTEILKGLKFNLTETSEAEIHQSFQHLLRTLNQSSDELQLSMGNAMFVKEQLSLLDRFTEDAK
RLYGSEAFATDFQDSAAAKKLINDYVKNGTRGKITDLIKDLDSQTMMVLVNYIFFKAKWEMPFDPQDTHQSRFYLSKKK
WVMVPMMSLHHLTIPYFRDEELSCTVVELKYTGNASALFILPDQDKMEEVEAMLLPETLKRWRDSLEFREIGELYLPKF
SISRDYNLNDILLQLGIEEAFTSKADLSGITGARNLAVSQVVHKAVLDVFEEGTEASAATAVKITLLSALVETRTIVRF
NRPFLMIIVPTDTQNIFFMSKVTNPKQA
Human SerpinA3
(SEQ ID NO: 13)
ATTCATGAAAATCCACTACTCCAGACAGACGGCTTTGGAATCCACCAGCTACATCCAGCTCCCTGAGGCA
GAGTTGAGAATGGAGAGAATGTTACCTCTCCTGGCTCTGGGGCTCTTGGCGGCTGGGTTCTGCCCTGCTG
TCCTCTGCCACCCTAACAGCCCACTTGACGAGGAGAATCTGACCCAGGAGAACCAAGACCGAGGGACACA
CGTGGACCTCGGATTAGCCTCCGCCAACGTGGACTTCGCTTTCAGCCTGTACAAGCAGTTAGTCCTGAAG
GCCCCTGATAAGAATGTCATCTTCTCCCCACTGAGCATCTCCACCGCCTTGGCCTTCCTGTCTCTGGGGG
CCCATAATACCACCCTGACAGAGATTCTCAAAGGCCTCAAGTTCAACCTCACGGAGACTTCTGAGGCAGA
AATTCACCAGAGCTTCCAGCACCTCCTGCGCACCCTCAATCAGTCCAGCGATGAGCTGCAGCTGAGTATG
GGAAATGCCATGTTTGTCAAAGAGCAACTCAGTCTGCTGGACAGGTTCACGGAGGATGCCAAGAGGCTGT
ATGGCTCCGAGGCCTTTGCCACTGACTTTCAGGACTCAGCTGCAGCTAAGAAGCTCATCAACGACTACGT
GAAGAATGGAACTAGGGGGAAAATCACAGATCTGATCAAGGACCTTGACTCGCAGACAATGATGGTCCTG
GTGAATTACATCTTCTTTAAAGCCAAATGGGAGATGCCCTTTGACCCCCAAGATACTCATCAGTCAAGGT
TCTACTTGAGCAAGAAAAAGTGGGTAATGGTGCCCATGATGAGTTTGCATCACCTGACTATACCTTACTT
CCGGGACGAGGAGCTGTCCTGCACCGTGGTGGAGCTGAAGTACACAGGCAATGCCAGCGCACTCTTCATC
CTCCCTGATCAAGACAAGATGGAGGAAGTGGAAGCCATGCTGCTCCCAGAGACCCTGAAGCGGTGGAGAG
ACTCTCTGGAGTTCAGAGAGATAGGTGAGCTCTACCTGCCAAAGTTTTCCATCTCGAGGGACTATAACCT
GAACGACATACTTCTCCAGCTGGGCATTGAGGAAGCCTTCACCAGCAAGGCTGACCTGTCAGGGATCACA
GGGGCCAGGAACCTAGCAGTCTCCCAGGTGGTCCATAAGGCTGTGCTTGATGTATTTGAGGAGGGCACAG
AAGCATCTGCTGCCACAGCAGTCAAAATCACCCTCCTTTCTGCATTAGTGGAGACAAGGACCATTGTGCG
TTTCAACAGGCCCTTCCTGATGATCATTGTCCCTACAGACACCCAGAACATCTTCTTCATGAGCAAAGTC
ACCAATCCCAAGCAAGCCTAGAGCTTGCCATCAAGCAGTGGGGCTCTCAGTAAGGAACTTGGAATGCAAG
CTGGATGCCTGGGTCTCTGGGCACAGCCTGGCCCCTGTGCACCGAGTGGCCATGGCATGTGTGGCCCTGT
CTGCTTATCCTTGGAAGGTGACAGCGATTCCCTGTGTAGCTCTCACATGCACAGGGGCCCATGGACTCTT
CAGTCTGGAGGGTCCTGGGCCTCCTGACAGCAATAAATAATTTCGTTGGAAAAAAAAAAAAAAAAAAAAA
AAAAAAAAAAAAAAAAAAA
Cowpox CrmA
(SEQ ID NO: 14)
MDIFREIASSMKGENVFISPPSISSVLTILYYGANGSTAEQLSKYVEKEADKNKDDISFKSMNKVYGRYSAVFKDSFLR
KIGDNFQTVDFTDCRTVDAINKCVDIFTEGKINPLLDEPLSPDTCLLAISAVYFKAKWLMPFEKEFTSDYPFYVSPTEM
VDVSMMSMYGEAFNHASVKESFGNFSIIELPYVGDTSMVVILPDNIDGLESIEQNLTDTNFKKWCDSMDAMFIDVHIPK
FKVTGSYNLVDALVKLGLTEVFGSTGDYSNMCNSDVSVDAMIHKTYIDVNEEYTEAAAATCALVADCASTVTNEFCADH
PFIYVIRHVDGKILFVGRYCSPTTN
Cowpox CrmA
(SEQ ID NO: 15)
TCCATGGAAGAACGAAAGTAGTATAAAAGTAATAAAACAAAAAAAAGAATATAAAAAATTTATAGCCACT
TTCTTTGAGGACTGTTTTCCTGAAGGAAATGAACCTCTGGAATTAGTTAGATATATAGAATTAGTATACA
CGCTAGATTATTCTCAAACTCCTAATTATGACAGACTACGTAGACTGTTTATACAAGATTGAAAATATAT
TTCTTTTTATTGAGTGGTGGTAGTTACGGATATCTAATATTAATATTAGACTATCTCTATCGTCACACAA
CAAAATCGATTGCCATGGATATCTTCAGGGAAATCGCATCTTCTATGAAAGGAGAGAATGTATTCATTTC
TCCACCGTCAATCTCGTCAGTATTGACAATACTGTATTATGGAGCTAATGGATCCACTGCTGAACAGCTA
TCAAAATATGTAGAAAAGGAGGCGGACAAGAATAAGGATGATATCTCATTCAAGTCCATGAATAAAGTAT
ATGGGCGATATTCTGCAGTGTTTAAAGATTCCTTTTTGAGAAAAATTGGAGATAATTTCCAAACTGTTGA
CTTCACTGATTGTCGCACTGTAGATGCGATCAACAAGTGTGTTGATATCTTCACTGAGGGGAAAATTAAT
CCACTATTGGATGAACCATTGTCTCCAGATACCTGTCTCCTAGCAATTAGTGCCGTATACTTTAAAGCAA
AATGGTTGATGCCATTTGAAAAGGAATTTACCAGTGATTATCCCTTTTACGTATCTCCAACGGAAATGGT
AGATGTAAGTATGATGTCTATGTACGGCGAGGCATTTAATCACGCATCTGTAAAAGAATCATTCGGCAAC
TTTTCAATCATAGAACTGCCATATGTTGGAGATACTAGTATGGTGGTAATTCTTCCAGACAATATTGATG
GACTAGAATCCATAGAACAAAATCTAACAGATACAAATTTTAAGAAATGGTGTGACTCTATGGATGCTAT
GTTTATCGATGTGCACATTCCCAAGTTTAAGGTAACAGGCTCGTATAATCTGGTGGATGCGCTAGTAAAG
TTGGGACTGACAGAGGTGTTCGGTTCAACTGGAGATTATAGCAATATGTGTAATTCAGATGTGAGTGTCG
ACGCTATGATCCACAAAACGTATATAGATGTCAATGAAGAGTATACAGAAGCAGCTGCAGCAACTTGTGC
GCTGGTGGCAGACTGTGCATCAACAGTTACAAATGAGTTCTGTGCAGATCATCCGTTCATCTATGTGATT
AGGCATGTCGATGGCAAAATTCTTTTCGTTGGTAGATATTGCTCTCCAACAACTAATTAAATCACATTCT
TAATATTAGAATATTAGAATATTATATAGTTAAGATTTTTACTAATTGGTTAACCATTTTTTTAAAAAAA
TAGAAAAAAAACATGTTATATTAGCGAGGGTCGTTATTCTTCCAATTGCAATTGGTAAGATGACGGCC
Human Serp2
(SEQ ID NO: 16)
MVAKQRIRMANEKHSKNITQRGNVAKTLRPQEEKYPVGPWLLALFVFVVCGSAIFQIIQSIRMGM
Human Serp2
(SEQ ID NO: 17)
GCCTCTCTCTGGAGTCGGCTAGCCGGGGCTCGGGGAGCGGGGTGCGCAGGGCTCGGGGCCACGCCTTGCC
ACCTGCAGCGCCCGGGTGGGCCGCGGGGGCCTCGGCGGGACGCGCTCGGCCCTGTCGCAGGAGCTAACGC
AGGGGGAATCCTTGCAGGTGGGAGCATTTCAGAGCGCACAAGCCATGGTGGCCAAACAGCGGATCCGGAT
GGCTAACGAGAAGCACAGCAAAAACATCACCCAGAGGGGGAACGTAGCCAAAACCCTGAGGCCGCAAGAG
GAGAAATATCCTGTGGGACCATGGCTGTTGGCACTGTTTGTTTTTGTTGTCTGTGGCTCAGCTATCTTTC
AGATCATTCAGAGCATAAGGATGGGCATGTGAGAAAGCCAGGGATTTGACACCACCTCCCTCCCACTGGA
GGCGGGAGGACAACGGAAGCGGTCAGCCAGTTTCTGCGGGAAACAAGCAGGCCACACGGAATAGAAAAAA
ACGCTCCCCCACTTGTTCCCTGATCACTTCATCGTGGATGTCAGACCAAATTGCCTTCTCACAGGACATC
TTGGTGCATCCGCGTTCTCAAGCGGAAAGGACATTTTGCTTTTCTGTTGGCAGGATTAGTAGCCACGCGG
GTCGTCCGCAGCAGTGCTGTCTTTTTGGTTTTTCCCTTGGTTTCACTAATGCGTGCATGTGGCCCTCTGA
ACGATCACTGGTTTACTTTCTATGGATACAATCTCTCCTCCATTGAGAATTGATTTTACAAATAAATGTC
TTCGTTCAACCTTAAAAAAAAAAAAAAAAAAAAAAAAAAAAAAAAAAAAAAAAAAAAAAAAAAAAAAAAA
AA
PI9 peptides
(SEQ ID NO: 18)
VEVNEEGTEAAAASSCFVVAECCMESGPRFCADHPFL
(SEQ ID NO: 19)
VEVNEEGTEAAAASSCFVVADCCMESGPRFCADHPFL
(SEQ ID NO: 20)
VEVNEEGTEAAAASSCFVVAACCMESGPRFCADHPFL
(SEQ ID NO: 21)
VEVNEEGREAAAASSCFVVAECCMESGPRFCADHPFL
CrmA peptide
(SEQ ID NO: 22)
IDVNEEYTEAAAATCALVADCASTVTNEFCADHPFI P

### REFERENCES

Adams and White. (2004) Eur. J. Pub. Health. 14(3):331-34.
Ang et al. (2008) Exp. Gerontol. 43:615-22.
Bhattacharyya, T. K. and Thomas, J. R. (2004) 6:21-5.
Bird et at (1998) Mol. Cell. Biol. 18, 6387-6398.
Buzza et at (2005) J. Biol. Chem. 280:23549-58.
Chamberlain et al. (2010) Am. J. Pathol. 176:1038-1049.
Choy et al. (2004) Arterioscler. Thromb. Vasc. Biol. 24(12):2245-50.
Creighton, T. E. Proteins: Structure and Molecular Properties, W.H. Freeman & Co. San Francisco, Calif., 1983).
Dallas et al. (1995) J. Cell. Biol. 131:539-549.
Feingold et al. (1995) J. Invest. Dermatol. 104:246-50.
Fleischmajer et al. (1991) J. Struct. Biol. 106:82-90.
Hildebrand et al. (1994) Biochem. J. 302(Pt2):527-534.
Hill et al. (1995) J. Thorac. Cardiovasc. Surg. 110:1658-1662.
Kam et al. (2000) Biochim. Biophys. Acta. 1477(1-2):307:23.
Kligman, J. H. (1996) Clin. Dermatol. 14:183-95.
Imai et al. (1997) Biochem. J. 322(Pt3):809-814.
Lopez-Casillas et al. (1994). J. Cell. Biol. 124:557-568.
Macri et al. (2007) Adv. Drug. Deliv. Rev. 59:1366-1381.
Mahrus and Craik. (2005) Chemistry & Biology 12:567-577.
Medema et al. (1997) Eur. J. Immunol. 27:3492-3498.
Pan and Clawson (2006) Curr. Med. Chem. 13(25): 3083-103.
Patzel (2007) Drug Discov. Today 12(3-4): 139-48.
Peek and Behlke (2007) Curr. Opin. Mol. Ther. 9(2): 110-8.
Prakash et al. (2008) Immunol Cell Biol. 87(3):249-54.
Raja et al. (2002) J. Biol. Chem. 277:49523-49530.
Schiller et al. (2004) J. Dermatol. Sci. 35:83-92.
Schonherr et al. (1995) J. Biol. Chem. 270:2776-2783
Sen et al. (2009) Wound Repair Regen. 17:763-71.
Sun et al. (1996) J. Biol. Chem. 271:27802-27809.
Sun et al. (1997) J. Biol. Chem. 272:15434-15441.
Taylor A W. (2009) J Leukoc Biol. 85:29-33.
Willoughby et al. (2002) Bioorg Med Chem Lett. 12:2197-2200.
Zipfel et al. (2003) Proc. Natl. Acad. Sci. U.S.A. 100:7075-80.

1. A method of promoting wound healing in a subject, the method comprising administering a Granzyme B inhibitor to the subject for a time and in an amount sufficient to promote wound healing, thereby promoting wound healing in the subject.

2. A method of promoting wound healing in a subject, the method comprising applying a Granzyme B (Granzyme B) inhibitor to the wound, for a time and in an amount sufficient to promote wound healing, thereby promoting wound healing in the subject.

3. The method of claim 1, wherein the wound is a chronic wound.

4. The method of claim 3, wherein the chronic wound is a chronic skin wound.

5. The method of claim 4, wherein the chronic skin wound is a pressure ulcer.

6. The method of claim 1, wherein cleavage of an extracellular matrix protein is inhibited.

7. The method of claim 6, wherein the extracellular matrix protein is selected from the group consisting of decorin, biglycan, betaglycan, syndecan, brevican, fibromodulin, fibrillin-1, fibrillin-2, and fibulin-2.

8. The method of claim 7, wherein the extracellular matrix protein is decorin.

9. The method of claim 1, wherein release of TGFβ bound to an extracellular matrix protein is inhibited.

10. The method of claim 9, wherein the extracellular matrix protein is decorin.

11. A method of preventing skin tearing of a subject, comprising applying a Granzyme B inhibitor to the skin of the subject for a time and in an amount sufficient to prevent skin tearing, thereby preventing skin tearing in the subject.

12. The method of claim 11, wherein the skin tearing is associated with a chronic wound.

13. The method of claim 11, wherein the skin tearing is associated with aging.

14-18. (canceled)

19. A method for inhibiting hypertrophic scarring of a wound, comprising applying a Granzyme B inhibitor to the skin of the subject for a time and in an amount sufficient to prevent skin hypertrophic scarring of a wound, thereby inhibiting hypertrophic scarring of a wound.

20. The method of claim 19, wherein cleavage of an extracellular matrix protein is inhibited.

21. The method of claim 20, wherein the extracellular matrix protein is selected from the group consisting of decorin, biglycan, betaglycan, syndecan, brevican, fibromodulin, fibrillin-1, fibrillin-2, and fibulin-2.

22. The method of claim 20, wherein the extracellular matrix protein is decorin.

23-24. (canceled)

25. A method for increasing collagen organization in the skin of a subject, comprising applying a Granzyme B inhibitor to the skin of the subject in an amount and for a time sufficient to increase collagen organization in the subject, thereby increasing collagen organization in the skin of the subject.

26-30. (canceled)

31. A method for increasing the tensile strength of a healing or healed skin wound of a subject, comprising applying a Granzyme B inhibitor to the skin of the subject in an amount and for a time sufficient to increase the tensile strength of the healing or healed skin wound of the subject, thereby increasing the tensile strength of a healing or healed skin wound of a subject.

32. The method of claim 31, wherein cleavage of an extracellular matrix protein is inhibited.

33. The method of claim 32, wherein the extracellular matrix protein is selected from the group consisting of decorin, biglycan, betaglycan, syndecan, brevican, fibromodulin, fibrillin-1, fibrillin-2, and fibulin-2.

34. The method of claim 32, wherein the extracellular matrix protein is decorin.

35. The method of claim 31, wherein release of TGFβ bound to an extracellular matrix protein is inhibited.

36. The method of claim 35, wherein the extracellular matrix protein is decorin.

37. A method for inhibiting release of TGFβ bound to an extracellular protein, comprising contacting the extracellular proteoglycan with a Granzyme B inhibitor, thereby inhibiting release of TGFβ bound to the extracellular protein.

38-39. (canceled)

40. A method of inhibiting extracellular decorin cleavage, comprising contacting decorin with a Granzyme B inhibitor, thereby inhibiting extracellular decorin cleavage.

41. The method of claim 1, wherein the Granzyme B inhibitor is selected from the group consisting of a nucleic acid molecule, a peptide, an antibody, and a small molecule.

42. The method of claim 41, wherein the antibody is a monoclonal antibody.

43. The method of claim 1, wherein the Granzyme B inhibitor is selected from one or more of the following:
(2S,5S)—N-((2H-tetrazol-5-yl)methyl)-5-((2S,3S)-acetamido-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((R)-3-methyl-2-(pyridin-2-yl)butanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide;
(2S,5S)—N-((1H-1,2,4-triazol-3-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-pyrazol-3-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-pyrazol-4-yl)methyl)-5-((2S,3S)-2-acetamido-3-methylpentanamido)-4-oxo-1,2,4,5,6,7-hexahydroazepino[3,2,1-hi]indole-2-carboxamide;
(2S,5S)—N-((1H-imidazol-4-yl)methyl)-5-((2S,3S)-2-acetamido-3-methylpentanamido)-4-oxo-1,2,4,5,6,7-hexahydroazepino[3,2,1-hi]indole-2-carboxamide;
2S,5S)-5-((2S,3S)-2-acetamido-3-methylpentanamido)-4-oxo-N-(thiazol-5-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(isoxazol-3-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-4-oxo-N-(thiazol-2-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(isoxazol-5-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-4-oxo-N-(thiazol-4-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyrimidin-5-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridazin-4-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-2-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-3-ylmethyl)-1,2,4,5,6,7-hexahydroazepino[3,2,1-hi]indole-2-carboxamide;
(2S,5S)-5-((2S,3S)-2-acetamido-3-methylpentanamido)-4-oxo-N-(pyridin-4-ylmethyl)-1,2,4,5,6,7-hexahydroazepino[3,2,1-hi]indole-2-carboxamide;
(2S,5S)-5-((2S,3S)-2-acetamido-3-methylpentanamido)-N-(imidazo[1,2-a]pyrimidin-2-ylmethyl)-4-oxo-1,2,4,5,6,7-hexahydroazepino[3,2,1-hi]indole-2-carboxamide;
(2S,5S)-5-((2S,3S)-2-acetamido-3-methylpentanamido)-N-((3a,7a-dihydrobenzo[d]thiazol-2-yl)methyl)-4-oxo-1,2,4,5,6,7-hexahydroazepino[3,2,1-hi]indole-2-carboxamide;
(2S,5S)—N-((2H-tetrazol-5-yl)methyl)-5-((R)-3-methyl-2-(pyridin-2-yl)butanamido)-4-oxo-1,2,4,5,6,7-hexahydroazepino[3,2,1-hi]indole-2-carboxamide;
(2S,5S)—N-((2H-tetrazol-5-yl)methyl)-5-((S)-3-methyl-2-(pyridin-2-yl)butanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-3-methyl-2-(2-phenylacetamido)pentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(2,3-difluorophenyl)acetamido)-3-methylpentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide;
(2S,5S)—N-((2H-tetrazol-5-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-(2-(dimethylamino)acetamido)-3-methylpentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-((2S,3S)-2-(2-(benzo[b]thiophen-3-yl)acetamido)-3-methylpentanamido)-4-oxo-1,2,4,5,6,7-hexahydroazepino[3,2,1-hi]indole-2-carboxamide;
(R)—N-((2S,5S)-2-((1H-1,2,3-triazol-4-yl)methylcarbamoyl)-4-oxo-1,2,4,5,6,7-hexahydroazepino[3,2,1-hi]indol-5-yl)-3-acetyl-5,5-dimethylthiazolidine-4-carboxamide;
(2S,5S)—N-((1H-1,2,3-triazol-4-yl)methyl)-5-((2S,3S)-3-methyl-2-(2-oxopyrrolidin-1-yl)pentanamido)-4-oxo-1,2,4,5,6,7-hexahydroazepino[3,2,1-hi]indole-2-carboxamide;
(2S,5S)—N-((1H-1,2,3-triazol-4-yl)methyl)-5-(2-cyclopentylacetamido)-4-oxo-1,2,4,5,6,7-hexahydroazepino[3,2,1-hi]indole-2-carboxamide;
(2S,5S)—N-((1H-1,2,3-triazol-4-yl)methyl)-5-((S)-2-acetamido-2-cyclopropylacetamido)-4-oxo-1,2,4,5,6,7-hexahydroazepino[3,2,1-hi]indole-2-carboxamide;
(2S,5S)—N-((1H-1,2,3-triazol-4-yl)methyl)-5-((S)-2-acetamido-2-cyclopentylacetamido)-4-oxo-1,2,4,5,6,7-hexahydroazepino[3,2,1-hi]indole-2-carboxamide;
Bio-x-IEPD^{P}-(OPh)₂;
azepino[3,2,1-hi]indole-2-carboxamide;
(4S)-4-[[(2S)-2-acetamido-4-methylpentanoyl]amino]-5-[2-[[(2S)-4-hydroxy-1,4-dioxobutan-2-yl]carbamoyl]pyrrolidin-1-yl]-5-oxopentanoic acid;
(4S)-4-[[(2S,3S)-2-acetamido-3-methylpentanoyl]amino]-5-[[(2S,3S)-3-hydroxy-1-[[(2S)-4-hydroxy-1,4-dioxobutan-2-yl]amino]-1-oxobutan-2-yl]amino]-5-oxopentanoic acid;
5-chloro-4-oxo-3-[2-[2-(phenylmethoxycarbonylamino) propanoylamino]propanoylamino]pentanoic acid;
5-chloro-4-oxo-2-[2-[2-(phenylmethoxycarbonylamino) propanoylamino]propanoylamino]pentanoic acid;
ZINC05723764 (NCI 644752);
ZINC05723787 (NCI 644777);
ZINC05316154 (NCI 641248);
ZINC05723499 (NCI 641235);
ZINC05723646 (NCI 642017);
ZINC05398428 (NCI 641230);
ZINC05723503 (NCI 641236);
ZINC05723446 (NCI 640985);
ZINC05317216 (NCI 618792);
ZINC05315460 (NCI 630295);
ZINC05316859 (NCI 618802);
ZINC05605947 (NCI 623744);
an isocoumarin;
a peptide chloromethyl ketone;
a peptide phosphonate;
a Granzyme B inhibitory nucleic acid molecule;
an anti-Granzyme B antibody;
an inhibitory Granzyme B peptide;
a SerpB9 polypeptide, or fragment thereof;
Ac-IEPD-CHO;
a Serp2 polypeptide, or a Granzyme B inhibitory fragment thereof;
a CrmA polypeptide or a Granzyme B inhibitory fragment thereof; and
a SerpinA3 polypeptide or a Granzyme B inhibitory fragment thereof.

44. The method of claim 1, wherein the Granzyme B inhibitor is formulated for topical administration.

45. The method of claim 1, wherein the Granzyme B inhibitor is formulated for co-administration with another wound treatment.

46. The method of claim 45, wherein another wound treatment is selected from one or more of: a topical antimicrobial; a cleanser, a wound gel; a collagen; an elastin; a tissue growth promoter; an enzymatic debriding preparation; an antifungal; an anti-inflammatory; a barrier; a moisturizer, and a sealant.

47. The method of claim 45, wherein the another wound treatment is a wound covering, a wound filler, or an implant.

48. The method of claim 45, wherein another wound treatment is an absorptive dressing; an alginate dressing; a foam dressing; a hydrocolloid dressing; a hydrofiber dressing; a compression dressing and wrap; a composite dressing; a contact layer; a wound gel impregnated gauze; a wound gel sheet; a transparent film; a wound filler; a dermal matrix product or a tissue scaffold; or a closure device.

49. The method of claim 1, wherein the subject is a mammal.

50. The method claim 1, wherein the subject is a human.

51-69. (canceled)
